SEQUENCE LISTING (48 sequences)

SEQ ID NO: 1 - DNA, 26 bp, Homo sapiens
atggtatccttacatcgtcatttatc

SEQ ID NO: 2 - DNA, 84 bp, Homo sapiens
cctctctatgggcagtcggtgattcctattcagatcacgatggtatccttacatctgagtcggagacacgcagggatgagatgg

SEQ ID NO: 3 - DNA, 78 bp, Homo sapiens
cctctctatgggcagtcggtgattcctattcagatggtatccttacatctgagtcggagacacgcagggatgagatgg

SEQ ID NO: 4 - DNA, 42 bp, Homo sapiens
cattcaaatccccgagttgaactttcctttcaaagttcacgt

SEQ ID NO: 5 - DNA, 25 bp, Homo sapiens
cgggaggcggaggttgcagtgagcc

SEQ ID NO: 6 - DNA, 17 bp, Homo sapiens
gtaaaacgacggccagt

SEQ ID NO: 7 - DNA, 17 bp, Homo sapiens
caggaaacagctatgac

SEQ ID NO: 8 - protein, 1210 aa, Homo sapiens [sequence omitted]

SEQ ID NO: 9 - protein, 5616 aa, Homo sapiens
CCCCGGCGCAGCGCGGCCGCAGCAGCCTCCGCCCCCCGCACGGTGTGAGCGCCCGACGCGGCCGAGGCGGCCGGAGTCCCGAGCTAGCCCCGGCGGCCGCCGCCGCCCAGACCGGACGACAGGCCACCTCGTCGGCGTCCGCCCGAGTCCCCGCCTCGCCGCCAACGCCACAACCACCGCGCACGGCCCCCTGACTCCGTCCAGTATTGATCGGGAGAGCCGGAGCGAGCTCTTCGGGGAGCAGCGATGCGACCCTCCGGGACGGCCGGGGCAGCGCTCCTGGCGCTGCTGGCTGCGCTCTGCCCGGCGAGTCGGGCTCTGGAGGAAAAGAAAGTTTGCCAAGGCACGAGTAACAAGCTCACGCAGTTGGGCACTTTTGAAGATCATTTTCTCAGCCTCCAGAGGATGTTCAATAACTGTGAGGTGGTCCTTGGGAATTTGGAAATTACCTATGTGCAGAGGAATTATGATCTTTCCTTCTTAAAGACCATCCAGGAGGTGGCTGGTTATGTCCTCATTGCCCTCAACACAGTGGAGCGAATTCCTTTGGAAAACCTGCAGATCATCAGAGGAAATATGTACTACGAAAATTCCTATGCCTTAGCAGTCTTATCTAACTATGATGCAAATAAAACCGGACTGAAGGAGCTGCCCATGAGAAATTTACAGGAAATCCTGCATGGCGCCGTGCGGTTCAGCAACAACCCTGCCCTGTGCAACGTGGAGAGCATCCAGTGGCGGGACATAGTCAGCAGTGACTTTCTCAGCAACATGTCGATGGACTTCCAGAACCACCTGGGCAGCTGCCAAAAGTGTGATCCAAGCTGTCCCAATGGGAGCTGCTGGGGTGCAGGAGAGGAGAACTGCCAGAAACTGACCAAAATCATCTGTGCCCAGCAGTGCTCCGGGCGCTGCCGTGGCAAGTCCCCCAGTGACTGCTGCCACAACCAGTGTGCTGCAGGCTGCACAGGCCCCCGGGAGAGCGACTGCCTGGTCTGCCGCAAATTCCGAGACGAAGCCACGTGCAAGGACACCTGCCCCCCACTCATGCTCTACAACCCCACCACGTACCAGATGGATGTGAACCCCGAGGGCAAATACAGCTTTGGTGCCACCTGCGTGAAGAAGTGTCCCCGTAATTATGTGGTGACAGATCACGGCTCGTGCGTCCGAGCCTGTGGGGCCGACAGCTATGAGATGGAGGAAGACGGCGTCCGCAAGTGTAAGAAGTGCGAAGGGCCTTGCCGCAAAGTGTGTAACGGAATAGGTATTGGTGAATTTAAAGACTCACTCTCCATAAATGCTACGAATATTAAACACTTCAAAAACTGCACCTCCATCAGTGGCGATCTCCACATCCTGCCGGTGGCATTTAGGGGTGACTCCTTCACACATACTCCTCCTCTGGATCCACAGGAACTGGATATTCTGAAAACCGTAAAGGAAATCACAGGGTTTTTGCTGATTCAGGCTTGGCCTGAAAACAGGACGGACCTCCATGCCTTTGAGAACCTAGAAATCATACGCGGCAGGACCAAGCAACATGGTCAGTTTTCTCTTGCAGTCGTCAGCCTGAACATAACATCCTTGGGATTACGCTCCCTCAAGGAGATAAGTGATGGAGATGTGATAATTTCAGGAAACAAAAATTTGTGCTATGCAAATACAATAAACTGGAAAAAACTGTTTGGGACCTCCGGTCAGAAAACCAAAATTATAAGCAACAGAGGTGAAAACAGCTGCAAGGCCACAGGCCAGGTCTGCCATGCCTTGTGCTCCCCCGAGGGCTGCTGGGGCCCGGAGCCCAGGGACTGCGTCTCTTGCCGGAATGTCAGCCGAGGCAGGGAATGCGTGGACAAGTGCAACCTTCTGGAGGGTGAGCCAAGGGAGTTTGTGGAGAACTCTGAGTGCATACAGTGCCACCCAGAGTGCCTGCCTCAGGCCATGAACATCACCTGCACAGGACGGGGACCAGACAACTGTATCCAGTGTGCCCACTACATTGACGGCCCCCACTGCGTCAAGACCTGCCCGGCAGGAGTCATGGGAGAAAACAACACCCTGGTCTGGAAGTACGCAGACGCCGGCCATGTGTGCCACCTGTGCCATCCAAACTGCACCTACGGATGCACTGGGCCAGGTCTTGAAGGCTGTCCAACGAATGGGCCTAAGATCCCGTCCATCGCCACTGGGATGGTGGGGGCCCTCCTCTTGCTGCTGGTGGTGGCCCTGGGGATCGGCCTCTTCATGCGAAGGCGCCACATCGTTCGGAAGCGCACGCTGCGGAGGCTGCTGCAGGAGAGGGAGCTTGTGGAGCCTCTTACACCCAGTGGAGAAGCTCCCAACCAAGCTCTCTTGAGGATCTTGAAGGAAACTGAATTCAAAAAGATCAAAGTGCTGGGCTCCGGTGCGTTCGGCACGGTGTATAAGGGACTCTGGATCCCAGAAGGTGAGAAAGTTAAAATTCCCGTCGCTATCAAGGAATTAAGAGAAGCAACATCTCCGAAAGCCAACAAGGAAATCCTCGATGAAGCCTACGTGATGGCCAGCGTGGACAACCCCCACGTGTGCCGCCTGCTGGGCATCTGCCTCACCTCCACCGTGCAGCTCATCACGCAGCTCATGCCCTTCGGCTGCCTCCTGGACTATGTCCGGGAACACAAAGACAATATTGGCTCCCAGTACCTGCTCAACTGGTGTGTGCAGATCGCAAAGGGCATGAACTACTTGGAGGACCGTCGCTTGGTGCACCGCGACCTGGCAGCCAGGAACGTACTGGTGAAAACACCGCAGCATGTCAAGATCACAGATTTTGGGCTGGCCAAACTGCTGGGTGCGGAAGAGAAAGAATACCATGCAGAAGGAGGCAAAGTGCCTATCAAGTGGATGGCATTGGAATCAATTTTACACAGAATCTATACCCACCAGAGTGATGTCTGGAGCTACGGGGTGACCGTTTGGGAGTTGATGACCTTTGGATCCAAGCCATATGACGGAATCCCTGCCAGCGAGATCTCCTCCATCCTGGAGAAAGGAGAACGCCTCCCTCAGCCACCCATATGTACCATCGATGTCTACATGATCATGGTCAAGTGCTGGATGATAGACGCAGATAGTCGCCCAAAGTTCCGTGAGTTGATCATCGAATTCTCCAAAATGGCCCGAGACCCCCAGCGCTACCTTGTCATTCAGGGGGATGAAAGAATGCATTTGCCAAGTCCTACAGACTCCAACTTCTACCGTGCCCTGATGGATGAAGAAGACATGGACGACGTGGTGGATGCCGACGAGTACCTCATCCCACAGCAGGGCTTCTTCAGCAGCCCCTCCACGTCACGGACTCCCCTCCTGAGCTCTCTGAGTGCAACCAGCAACAATTCCACCGTGGCTTGCATTGATAGAAATGGGCTGCAAAGCTGTCCCATCAAGGAAGACAGCTTCTTGCAGCGATACAGCTCAGACCCCACAGGCGCCTTGACTGAGGACAGCATAGACGACACCTTCCTCCCAGTGCCTGAATACATAAACCAGTCCGTTCCCAAAAGGCCCGCTGGCTCTGTGCAGAATCCTGTCTATCACAATCAGCCTCTGAACCCCGCGCCCAGCAGAGACCCACACTACCAGGACCCCCACAGCACTGCAGTGGGCAACCCCGAGTATCTCAACACTGTCCAGCCCACCTGTGTCAACAGCACATTCGACAGCCCTGCCCACTGGGCCCAGAAAGGCAGCCACCAAATTAGCCTGGACAACCCTGACTACCAGCAGGACTTCTTTCCCAAGGAAGCCAAGCCAAATGGCATCTTTAAGGGCTCCACAGCTGAAAATGCAGAATACCTAAGGGTCGCGCCACAAAGCAGTGAATTTATTGGAGCATGACCACGGAGGATAGTATGAGCCCTAAAAATCCAGACTCTTTCGATACCCAGGACCAAGCCACAGCAGGTCCTCCATCCCAACAGCCATGCCCGCATTAGCTCTTAGACCCACAGACTGGTTTTGCAACGTTTACACCGACTAGCCAGGAAGTACTTCCACCTCGGGCACATTTTGGGAAGTTGCATTCCTTTGTCTTCAAACTGTGAAGCATTTACAGAAACGCATCCAGCAAGAATATTGTCCCTTTGAGCAGAAATTTATCTTTCAAAGAGGTATATTTGAAAAAAAAAAAAAGTATATGTGAGGATTTTTATTGATTGGGGATCTTGGAGTTTTTCATTGTCGCTATTGATTTTTACTTCAATGGGCTCTTCCAACAAGGAAGAAGCTTGCTGGTAGCACTTGCTACCCTGAGTTCATCCAGGCCCAACTGTGAGCAAGGAGCACAAGCCACAAGTCTTCCAGAGGATGCTTGATTCCAGTGGTTCTGCTTCAAGGCTTCCACTGCAAAACACTAAAGATCCAAGAAGGCCTTCATGGCCCCAGCAGGCCGGATCGGTACTGTATCAAGTCATGGCAGGTACAGTAGGATAAGCCACTCTGTCCCTTCCTGGGCAAAGAAGAAACGGAGGGGATGGAATTCTTCCTTAGACTTACTTTTGTAAAAATGTCCCCACGGTACTTACTCCCCACTGATGGACCAGTGGTTTCCAGTCATGAGCGTTAGACTGACTTGTTTGTCTTCCATTCCATTGTTTTGAAACTCAGTATGCTGCCCCTGTCTTGCTGTCATGAAATCAGCAAGAGAGGATGACACATCAAATAATAACTCGGATTCCAGCCCACATTGGATTCATCAGCATTTGGACCAATAGCCCACAGCTGAGAATGTGGAATACCTAAGGATAGCACCGCTTTTGTTCTCGCAAAAACGTATCTCCTAATTTGAGGCTCAGATGAAATGCATCAGGTCCTTTGGGGCATAGATCAGAAGACTACAAAAATGAAGCTGCTCTGAAATCTCCTTTAGCCATCACCCCAACCCCCCAAAATTAGTTTGTGTTACTTATGGAAGATAGTTTTCTCCTTTTACTTCACTTCAAAAGCTTTTTACTCAAAGAGTATATGTTCCCTCCAGGTCAGCTGCCCCCAAACCCCCTCCTTACGCTTTGTCACACAAAAAGTGTCTCTGCCTTGAGTCATCTATTCAAGCACTTACAGCTCTGGCCACAACAGGGCATTTTACAGGTGCGAATGACAGTAGCATTATGAGTAGTGTGGAATTCAGGTAGTAAATATGAAACTAGGGTTTGAAATTGATAATGCTTTCACAACATTTGCAGATGTTTTAGAAGGAAAAAAGTTCCTTCCTAAAATAATTTCTCTACAATTGGAAGATTGGAAGATTCAGCTAGTTAGGAGCCCACCTTTTTTCCTAATCTGTGTGTGCCCTGTAACCTGACTGGTTAACAGCAGTCCTTTGTAAACAGTGTTTTAAACTCTCCTAGTCAATATCCACCCCATCCAATTTATCAAGGAAGAAATGGTTCAGAAAATATTTTCAGCCTACAGTTATGTTCAGTCACACACACATACAAAATGTTCCTTTTGCTTTTAAAGTAATTTTTGACTCCCAGATCAGTCAGAGCCCCTACAGCATTGTTAAGAAAGTATTTGATTTTTGTCTCAATGAAAATAAAACTATATTCATTTCCACTCTAAAAAAAAAAAAAAAAA

SEQ ID NO: 10 - protein, 1255 aa, Homo sapiens [sequence omitted]

SEQ ID NO: 11 - DNA, 4624 bp, Homo sapiens
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 12 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 13 - DNA, 5765 bp, Homo sapiens
actccagcctcgcgcgggagggggcgcggccgtgactcacccccttccctctgcgttcctccctccctctctctctctctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatccagcagtgagaagcttccaggtaggacagaaaaaagatccagcttcagctgcacacctctgtccccttggatggggaactaagggaaaacgtctgttgtatcactgaagttttttgttttgtttttatacgtgtctgaataaaaatgccaaagttttttttcagcaaaaaaaa

SEQ ID NO: 14 - protein, 1282 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCIGSSIEDCIGLMDRTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 15 - DNA, 3931 bp, Homo sapiens
cagcacgggatctgagacttccaaaaaatgaagccggcgacaggactttgggtctgggtgagccttctcgtggcggcggggaccgtccagcccagcgattctcagtcagtgtgtgcaggaacggagaataaactgagctctctctctgacctggaacagcagtaccgagccttgcgcaagtactatgaaaactgtgaggttgtcatgggcaacctggagataaccagcattgagcacaaccgggacctctccttcctgcggtctgttcgagaagtcacaggctacgtgttagtggctcttaatcagtttcgttacctgcctctggagaatttacgcattattcgtgggacaaaactttatgaggatcgatatgccttggcaatatttttaaactacagaaaagatggaaactttggacttcaagaacttggattaaagaacttgacagaaatcctaaatggtggagtctatgtagaccagaacaaattcctttgttatgcagacaccattcattggcaagatattgttcggaacccatggccttccaacttgactcttgtgtcaacaaatggtagttcaggatgtggacgttgccataagtcctgtactggccgttgctggggacccacagaaaatcattgccagactttgacaaggacggtgtgtgcagaacaatgtgacggcagatgctacggaccttacgtcagtgactgctgccatcgagaatgtgctggaggctgctcaggacctaaggacacagactgctttgcctgcatgaatttcaatgacagtggagcatgtgttactcagtgtccccaaacctttgtctacaatccaaccacctttcaactggagcacaatttcaatgcaaagtacacatatggagcattctgtgtcaagaaatgtccacataactttgtggtagattccagttcttgtgtgcgtgcctgccctagttccaagatggaagtagaagaaaatgggattaaaatgtgtaaaccttgcactgacatttgcccaaaagcttgtgatggcattggcacaggatcattgatgtcagctcagactgtggattccagtaacattgacaaattcataaactgtaccaagatcaatgggaatttgatctttctagtcactggtattcatggggacccttacaatgcaattgaagccatagacccagagaaactgaacgtctttcggacagtcagagagataacaggtttcctgaacatacagtcatggccaccaaacatgactgacttcagtgttttttctaacctggtgaccattggtggaagagtactctatagtggcctgtccttgcttatcctcaagcaacagggcatcacctctctacagttccagtccctgaaggaaatcagcgcaggaaacatctatattactgacaacagcaacctgtgttattatcataccattaactggacaacactcttcagcacaatcaaccagagaatagtaatccgggacaacagaaaagctgaaaattgtactgctgaaggaatggtgtgcaaccatctgtgttccagtgatggctgttggggacctgggccagaccaatgtctgtcgtgtcgccgcttcagtagaggaaggatctgcatagagtcttgtaacctctatgatggtgaatttcgggagtttgagaatggctccatctgtgtggagtgtgacccccagtgtgagaagatggaagatggcctcctcacatgccatggaccgggtcctgacaactgtacaaagtgctctcattttaaagatggcccaaactgtgtggaaaaatgtccagatggcttacagggggcaaacagtttcattttcaagtatgctgatccagatcgggagtgccacccatgccatccaaactgcacccaagggtgcataggctcaagtattgaagactgcatcggcctgatggatagaactcccctgattgcagctggagtaattggtgggctcttcattctggtcattgtgggtctgacatttgctgtttatgttagaaggaagagcatcaaaaagaaaagagccttgagaagattcttggaaacagagttggtggaaccattaactcccagtggcacagcacccaatcaagctcaacttcgtattttgaaagaaactgagctgaagagggtaaaagtccttggctcaggtgcttttggaacggtttataaaggtatttgggtacctgaaggagaaactgtgaagattcctgtggctattaagattcttaatgagacaactggtcccaaggcaaatgtggagttcatggatgaagctctgatcatggcaagtatggatcatccacacctagtccggttgctgggtgtgtgtctgagcccaaccatccagctggttactcaacttatgccccatggctgcctgttggagtatgtccacgagcacaaggataacattggatcacaactgctgcttaactggtgtgtccagatagctaagggaatgatgtacctggaagaaagacgactcgttcatcgggatttggcagcccgtaatgtcttagtgaaatctccaaaccatgtgaaaatcacagattttgggctagccagactcttggaaggagatgaaaaagagtacaatgctgatggaggaaagatgccaattaaatggatggctctggagtgtatacattacaggaaattcacccatcagagtgacgtttggagctatggagttactatatgggaactgatgacctttggaggaaaaccctatgatggaattccaacgcgagaaatccctgatttattagagaaaggagaacgtttgcctcagcctcccatctgcactattgacgtttacatggtcatggtcaaatgttggatgattgatgctgacagtagacctaaatttaaggaactggctgctgagttttcaaggatggctcgagaccctcaaagatacctagttattcagggtgatgatcgtatgaagcttcccagtccaaatgacagcaagttctttcagaatctcttggatgaagaggatttggaagatatgatggatgctgaggagtacttggtccctcaggctttcaacatcccacctcccatctatacttccagagcaagaattgactcgaataggaaccagtttgtgtaccgagatggaggttttgctgctgaacaaggagtgtctgtgccctacagagccccaactagcacaattccagaagctcctgtggcacagggtgctactgctgagatttttgatgactcctgctgtaatggcaccctacgcaagccagtggcaccccatgtccaagaggacagtagcacccagaggtacagtgctgaccccaccgtgtttgccccagaacggagcccacgaggagagctggatgaggaaggttacatgactcctatgcgagacaaacccaaacaagaatacctgaatccagtggaggagaacccttttgtttctcggagaaaaaatggagaccttcaagcattggataatcccgaatatcacaatgcatccaatggtccacccaaggccgaggatgagtatgtgaatgagccactgtacctcaacacctttgccaacaccttgggaaaagctgagtacctgaagaacaacatactgtcaatgccagagaaggccaagaaagcgtttgacaaccctgactactggaaccacagcctgccacctcggagcacccttcagcacccagactacctgcaggagtacagcacaaaatatttttataaacagaatgggcggatccggcctattgtggcagagaatcctgaatacctctctgagttctccctgaagccaggcactgtgctgccgcctccaccttacagacaccggaatactgtggtgtaagctcagttgtggttttttaggtggagagacacacctgctccaatttccccacccc

SEQ ID NO: 16 - protein, 595 aa, Homo sapiens [sequence omitted]

SEQ ID NO: 17 - DNA, 6330 bp, Homo sapiens
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 18 - protein, 932 aa, Homo sapiens
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPLGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHK

SEQ ID NO: 19 - DNA, 3014 bp, Homo sapiens
ctgaccagcgccgccctcccccgcccccgacccaggaggtggagatccctccggtccagccacattcaacacccactttctcctccctctgcccctatattcccgaaaccccctcctccttcccttttccctcctccctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccattgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacatgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtgg

SEQ ID NO: 20 - DNA, 31 bp, Homo sapiens
tctagccatttgatgccaacagtagaaaggg

SEQ ID NO: 21 - DNA, 19 bp, Homo sapiens
cttgactgagcgactgagc

SEQ ID NO: 22 - DNA, 19 bp, Homo sapiens
ctccagggttaggcagatc

SEQ ID NO: 23 - DNA, 19 bp, Homo sapiens
cagcgggatagtgcgattc

SEQ ID NO: 24 - DNA, 22 bp, Homo sapiens
taacttacggagtcgctctacg

SEQ ID NO: 25 - DNA, 22 bp, Homo sapiens
ggatgggattctttaggtcctg

SEQ ID NO: 26 - DNA, 58 bp, Homo sapiens
aatgatacggcgaccaccgagatctacactctttccctacacgacgctcttccgatct

SEQ ID NO: 27 - DNA, 61 bp, Homo sapiens
caagcagaagacggcatacgagatcggtctcggcattcctgctgaaccgctcttccgatct

SEQ ID NO: 28 - DNA, 17 bp, Homo sapiens
cgaccaccgagatctac

SEQ ID NO: 29 - DNA, 17 bp, Homo sapiens
acgtgaactttgaaagg

SEQ ID NO: 30 - DNA, 21 bp, Homo sapiens
gtagatcctgcaaaaagagtg

SEQ ID NO: 31 - DNA, 19 bp, Homo sapiens
cctttctactgttggcatc

SEQ ID NO: 32 - DNA, 20 bp, Homo sapiens
ctgagaatgattctgtctgg

SEQ ID NO: 33 - DNA, 19 bp, Homo sapiens
caatgcggtccatatatcc

SEQ ID NO: 34 - DNA, 42 bp, Homo sapiens; identical to SEQ ID NO: 4

SEQ ID NO: 35 - DNA, 31 bp, Homo sapiens; identical to SEQ ID NO: 20

SEQ ID NO: 36 - DNA, 64 bp, Homo sapiens
taacttacggagtcgctctacgcattcaaatccccgagttgaactttcctttcaaagttcacgt

SEQ ID NO: 37 - DNA, 53 bp, Homo sapiens
taacttacggagtcgctctacgtctagccatttgatgccaacagtagaaaggg

SEQ ID NO: 38 - DNA, 64 bp, Homo sapiens; identical to SEQ ID NO: 36

SEQ ID NO: 39 - DNA, 65 bp, Homo sapiens
taacttacggagtcgctctacgcattcaaatccccgagttgaactttcctttcaaagttcacgtt

SEQ ID NO: 40 - DNA, 58 bp, Homo sapiens
taacttacggagtcgctctacgtctagccatttgatgccaacagtagaaagggaaata

SEQ ID NO: 41 - DNA, 62 bp, Homo sapiens
taacttacggagtcgctctacgtctagccatttgatgccaacagtagaaagggaaatatctt

SEQ ID NO: 42 - DNA, 22 bp, Homo sapiens
taacttacggagtcgctctacg

SEQ ID NO: 43 - DNA, 30 bp, Homo sapiens
tctagccatttgatgccaacagtagaaagg

SEQ ID NO: 44 - DNA, 53 bp, Homo sapiens; identical to SEQ ID NO: 37

SEQ ID NO: 45 - DNA, 89 bp, Homo sapiens
aatgatacggcgaccaccgagatctacactctttccctacacgacgctcttccgatcttctagccatttgatgccaacagtagaaaggg

SEQ ID NO: 46 - DNA, 100 bp, Homo sapiens
aatgatacggcgaccaccgagatctacactctttccctacacgacgctcttccgatctcattcaaatccccgagttgaactttcctttcaaagttcacgt

SEQ ID NO: 47 - DNA, 55 bp, Homo sapiens
taacttacggagtcgctctacggcatggagcacttgcgagaggtgagggcagtta

SEQ ID NO: 48 - DNA, 60 bp, Homo sapiens
taacttacggagtcgctctacgagtggtgtgagggctccagcttgtatcaccatctattg

(19) United States
(12) Patent Application Publication
(10) Pub. No.: US 2016/0362730 A1
(43) Pub. Date: Dec. 15, 2016

(54) PHOTO-SELECTIVE METHOD FOR BIOLOGICAL SAMPLE ANALYSIS

(71) Applicant: Ventana Medical Systems, Inc., Tucson, AZ (US)

(72) Inventors: Nelson Alexander, Marana, AZ (US); Larry Morrison, Oro Valley, AZ (US); Sedide Ozturk, Oro Valley, AZ (US)

(21) Appl. No.: 15/249,326

(22) Filed: Aug. 26, 2016

Related U.S. Application Data
(63) Continuation of application No. PCT/EP2015/053681, filed on Feb. 23, 2015.
(60) Provisional application No. 61/944,996, filed on Feb. 26, 2014.

(51) Int. Cl. C12Q 1/68

(52) U.S. Cl. CPC C12Q 1/6823

(57) ABSTRACT

The present disclosure concerns novel methods for analyzing biological samples using photo-cleavable moieties that facilitate target detection and/or biomarker isolation. In some embodiments, the method concerns using a probe comprising a photo-cleavable moiety and a selective irradiation technique for activating/cleaving the photo-cleavable moiety thereby providing facile isolation of the probe or probe components. Also disclosed herein is a system and kit for implementing the methods disclosed herein.

### CROSS REFERENCE TO RELATED APPLICATIONS

This patent application is a continuation of International Application No. PCT/EP2015/053681 filed Feb. 23, 2015, which claims priority to and the benefit of U.S. Provisional Patent Application No. 61/944,996, filed Feb. 26, 2014. Each patent application is incorporated herein by reference as if set forth in its entirety.

### SEQUENCE LISTING

The sequence listing entitled “P32014_sequence_listing_ST25.txt,” which was created on 25 Aug. 2016 and has a size of 144,719 bytes, filed herewith, is incorporated-by-reference.

### FIELD

The present disclosure concerns embodiments of a method for photo-selective detection and/or isolation of a target, as well as probes and assays suitable for practicing these embodiments for biological analysis.

### BACKGROUND

Detecting proteins from formalin-fixed, paraffin-embedded (FFPE) tissue in a multiplexed and quantifiable fashion is an important aspect of tissue diagnostics. Current methods, such as immunohistochemistry, have low sensitivities, narrow dynamic ranges, and limited multiplexing capabilities. Unlike nucleic acids, proteins cannot be amplified; therefore, multiplexed detection techniques and quantifying low expressing protein targets from FFPE tissue can be difficult. Also, proteins of interest are typically expressed in a sample over a wide range of locations, making simultaneous detection of multiple targets from the same tissue sample even more difficult. Highly multiplexed tests, which are useful for detecting multiple targets in a single sample (e.g., a tissue sample), require sample isolation. Lysing is a typical method used to remove testable analytes from the tissue for analysis. Typical lysing methods, such as macroscopic dissection and laser capture microdissection (LCM), can be problematic as these methods disrupt overall tissue context, can cause tissue destruction, and/or require physically removing sections of the tissue from a sample. Multiplexing assays in tissue samples also are limited to analyzing a limited number of targets due to the probes and labels used in these types of assays.

Chromosomal alterations within individual cells also can be analyzed using in situ hybridization techniques (ISH). Currently, these analyses are limited to aberrations larger than whole genes. Particular targets or modifications (e.g., SNPs, promoter methylation, miRNA, and structural alterations substantially smaller than 100 kb) cannot be detected using ISH. Reference is made to “Cancer Cell Profiling by Barcoding Allows Multiplexed Protein Analysis in Fine-Needle Aspirates” published in Science Translational Medicine, by A. V. Ullal et al. (2014), which is hereby incorporated by reference, in its entirety, for disclosure related to multiplexed oncology diagnostics. In particular, the reference describes the importance of multiplexed diagnostic methods to expand diagnostic capability in oncology. While that disclosure provides robust analysis of the medical value provided by highly multiplexed assays, the samples were limited to fine needle aspirates which are divorced from contextual information as described herein. A need exists in the art for biological assays that produce quantifiable data and that can combine sensitive and multiplexed target analysis using next generation sequencing, PCR, and/or array technology in a variety of biological samples.

### SUMMARY

Disclosed herein are probes for analyzing a biological sample, and methods of using such probes. The methods disclosed herein can be used to perform contextual molecular diagnostics of various different types of samples. In one disclosed method, contextual molecular diagnostics is performed by contacting a sample with a probe comprising a photo-cleavable moiety, a specific binding moiety, and a detection tag using conditions sufficient to facilitate binding of the specific binding moiety to a target in the sample; removing probe that does not bind to the sample; selecting a region of the sample for irradiation based on contextual information; irradiating the selected region of the sample with light of a wavelength and an intensity sufficient to cleave the photo-cleavable moiety, thereby freeing the detection tag from the selected region of the sample; and detecting the detection tag. This method may further include contacting that includes multiple probes having specific binding moieties to multiple targets, the multiple different probes comprising unique detection tags and unique specific binding moieties and detecting the detection tag includes detecting the multiple unique detection tags. Illustratively, the method may further include selecting additional regions so that the method further includes iteratively and separately irradiating the additional regions. The method may further include iteratively depositing the detection tags from the additional regions in additional separate reaction vials. The additional separate reaction vials may contain PCR primers with distinct ID sequences. As such, the method may include appending the PCR primers to the detection tags. Illustratively, the method may include iteratively and separately detecting the detection tag from the additional selected regions. The method may further comprise relating the detecting of the detection tag from the selected regions to the sample so as to create a molecular profile of the sample, wherein the molecular profile relates each selected area with the detection tags detected from the selected area across the sample.

In another disclosed method, contextual molecular diagnostics can be performed by contacting a sample with a probe comprising a photo-reactive moiety, a specific binding moiety, and a detection tag using conditions sufficient to facilitate binding of the specific binding moiety to a target in the sample; removing probe that does not bind to the sample; selecting a region of the sample for irradiation based on contextual information; irradiating the selected region of the sample with light of a wavelength and an intensity sufficient to cause the photo-reactive moiety to react, thereby freeing the detection sequence for further reaction; exposing the tissue sample to an enzyme that acts on the detection oligonucleotide to effect a change; and detecting the change. In one embodiment, the detection tag is a polymerase chain reaction primer, the photo-reactive moiety is a photo-cleavable blocking group, the enzyme is a polymerase, and the change is an extension of the primer sequence. In another embodiment, the method further includes ligating adjacent detection tags after irradiating the selected region.

In a disclosed system for performing contextual diagnostics using a photo-activated probe, the system includes a slide configured to accept a sample; a vessel containing the photo-activated probe; a slide imaging device configured to receive the slide, and a light source configured to selectively irradiate the sample. The photo-activated probe comprises a specific binding moiety, a photo-activated moiety, and a detection tag, wherein the photo-activated probe is configured to be detectable upon selective irradiation. In a disclosed kit for performing contextual diagnostics, the kit includes a probe reagent solution comprising a specific binding moiety, a photo-cleavable moiety, and a detection tag.

The foregoing and other objects, features, and advantages of the invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1(A) is a schematic drawing of a probe showing a specific binding moiety and a detection tag coupled through a photo-reactive linker.

FIG. 1(B) is a schematic drawing of a probe in which the specific binding moiety is an anti-body.

FIG. 1(C) is a schematic drawing of a probe in which the specific binding moiety is an oligonucleotide/polynucleotide.

FIG. 1(D) is a schematic drawing of a specific binding moiety, a detection tag, and a photo-reactive moiety coupled thereto.

FIG. 1(E) is a schematic drawing of a probe in which the specific binding moiety is an oligonucleotide/polynucleotide.

FIG. 2(A) is a schematic representation of a detection tag with various identification components coupled to a photo-cleavable moiety.

FIG. 2(B) is a sequence (SEQ ID NO: 1) representation of a detection tag showing exemplary identification parts.

FIG. 3 is a schematic drawing showing a method of performing contextual molecular diagnostics.

FIG. 4 is a photomicrograph showing an approach to selecting two regions of a sample.

FIG. 5(A) is schematic drawing showing the use of a microscope objective to selectively irradiate a region of a sample.

FIG. 5(B) is schematic drawing showing the use of a liquid crystal screen to selectively irradiate a region of a sample.

FIG. 5(C) is schematic drawing showing the use of a digital micromirror device to selectively irradiate a region of a sample.

FIG. 6(A) is schematic drawings illustrating a probe bound to a sample prior to exposure to a light source.

FIG. 6(B) is an embodiment of a disclosed method illustrating an exposure step.

FIG. 6(C) is an embodiment of a disclosed method illustrating subsequent probe extension.

FIG. 6(D) is an embodiment of a disclosed method illustrating an extended probe being subjected to next generation sequencing.

FIG. 6(E) is an embodiment of a disclosed method illustrating an alternative light-activation approach analogous to FIG. 6(A)-(B).

FIG. 6(F) is an embodiment of a disclosed method illustrating an alternative light-activation approach analogous to FIG. 6(A)-(B).

FIG. 7 is a schematic drawing illustrating an embodiment of a disclosed method whereby a probe comprising an oligo/polynucleotide, a photo-cleavable moiety, and a tag sequence modified with a detectable label is added to a sample and irradiated with light.

FIG. 8 is a schematic drawing illustrating an embodiment of a disclosed method whereby a caged ATP moiety is used so that irradiating with light liberates the caged ATP and enables detection.

FIG. 9 is a schematic drawing illustrating an embodiment of a disclosed method whereby a ligase joins two different adjacent probes bound to targets within a sample.

FIG. 10 is a schematic drawing illustrating a method for performing contextual tissue diagnostics which shows iterative irradiation of a sample and separate collection of detection tags therefrom into distinct vessels, which may or may not include location tags.

FIG. 11 is a photographic image illustrating results obtained from a control sample used in an embodiment of a disclosed method.

FIG. 12 is a photographic image illustrating selective irradiation of a sample.

FIG. 13 is a photographic image illustrating selective irradiation of a sample.

FIG. 14 is an exploded view of the photographic image of FIG. 13, further illustrating the difference in signal obtained from a sample that has been selectively irradiated.

FIG. 15 is a photographic image of a patterned sample obtained by selectively irradiating a slide with a digital micromirror device and a patterned mask.

FIG. 16 is an exploded view of the image illustrated in FIG. 15.

FIG. 17 is an image of an electrophoresis gel illustrating the detection of unique sequence identifiers after photoselective cleavage from a specific binding moiety.

FIG. 18 is a schematic drawing illustrating an in situ polymerase extension embodiment whereby reverse primers are used to detect regions of the extended probe that are further away from the initial probe sequence.

FIG. 19 is an image of an electrophoresis gel analyzing isolated sequences obtained using in situ polymerase extension.

FIG. 20 is an image of an electrophoresis gel analyzing isolated CHR17 sequences obtained using in situ polymerase extension.

FIG. 21 is an image of an electrophoresis gel analyzing isolated sequences obtained from extension of two different probes within the same sample using in situ polymerase extension.

FIG. 22(A) a schematic drawing illustrating an in situ polymerase extension embodiment.

FIG. 22(B) is an image of an electrophoresis gel analyzing isolated sequences therefrom.

FIG. 23(A) is an image of an electrophoresis gel analyzing isolated sequences obtained for BRAF Exon 13.

FIG. 23(B) is an image of an electrophoresis gel analyzing isolated sequences obtained for HER2 Exon 10 (B) probes extended with and without caged thymidines.

### DETAILED DESCRIPTION

### I. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B. Nucleotide sizes or amino acid sizes, and molecular weight or molecular mass values, stated for nucleic acids or polypeptides or other compounds are generally approximate, and are provided for description.

Concentrations, amounts, and other numerical data may be expressed or presented herein as a range. Such ranges are used merely for convenience and brevity and should be interpreted to include not only the numerical values explicitly recited as the range limits, but also to include all individual numerical values or sub-ranges encompassed within that range as if each numerical value and sub-range is explicitly recited. As an illustration, a numerical range of “about 1 to about 5” includes not only the explicitly recited values of about 1 to about 5, but also values and sub-ranges within the indicated range, such as 2, 3, and 4 and sub-ranges such as from 1-3, from 2-4, and from 3-5, etc., as well as 1, 2, 3, 4, and 5 individually. This same principle applies to ranges that recite only one numerical value as a minimum or a maximum. Furthermore, such an interpretation should apply regardless of the breadth of the range or the characteristics being described.

Although methods and materials similar or equivalent to those described herein can be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

The following explanations of specific terms are provided to facilitate review of the various disclosed embodiments:

“Antibody” occasionally abbreviated “Ab,” refers to immunoglobulins or immunoglobulin-like molecules (including by way of example and without limitation, IgA, IgD, IgE, IgG and IgM, combinations thereof), similar molecules produced during an immune response, (e.g., in mammals such as humans, goats, rabbits and mice) as well as antibody fragments, that specifically bind to a molecule of interest (or a group of highly similar molecules of interest) to the substantial exclusion of binding to other molecules. For example, antibodies and antibody fragments typically have a binding constant for the molecule of interest that is at least 10³ M⁻¹ greater, at least 10⁴ M⁻¹ greater, or at least 10⁵ M⁻¹ greater than a binding constant for other molecules in a biological sample. “Antibody” further refers to a polypeptide ligand comprising at least a light chain or heavy chain immunoglobulin variable region that specifically recognizes and binds an epitope of an antigen. Antibodies may comprise of a heavy and a light chain, each of which has a variable region, termed the variable heavy (VH) region and the variable light (VL) region. Together, the VH region and the VL region are responsible for binding an antigen recognized by the antibody. “Antibody” also includes intact immunoglobulins and the variants and portions of them well known in the art. Antibody fragments include proteolytic antibody fragments, such as F(ab′)2 fragments, Fab′ fragments, Fab′-SH fragments and Fab fragments as are known in the art; recombinant antibody fragments, such as sFv fragments, dsFv fragments, bispecific sFv fragments, bispecific dsFv fragments, F(ab)′2 fragments; single chain Fv proteins (“scFv”); disulfide stabilized Fv proteins (“dsFv”); diabodies; triabodies (as are known in the art); and camelid antibodies (see, for example, U.S. Pat. Nos. 6,015,695; 6,005,079, 5,874,541; 5,840,526; 5,800,988; and 5,759,808). “Antibody” also includes monoclonal antibodies, which are characterized by being produced by a single clone of B lymphocytes or by a cell into which the light and heavy chain genes of a single antibody have been transfected. Monoclonal antibodies are produced by methods known to those of skill in the art. Monoclonal antibodies include humanized monoclonal antibodies.

“Conjugate” refers to two or more moieties directly or indirectly coupled together. For example, a first moiety may be covalently or noncovalently (e.g., electrostatically) coupled to a second moiety. Indirect attachment is possible, such as by using a linker, which may be a molecule or group of atoms positioned between two moieties.

“Contacting” refers to placement that allows association between two or more moieties, particularly direct physical association, for example both in solid form and/or in liquid form (for example, the placement of a biological sample, such as a biological sample affixed to a slide, in contact with a composition, such as a solution containing the probes disclosed herein).

“Context, -ual” refers to the environment associated with a sample, wherein the integrity of, or information obtained from, a certain portion of the sample can be determined by comparing it with the entire sample environment. Contextual information is that information that can be understood by considering a sample's spatial and relational characteristics. As an example, contextual information for a tissue sample would include the type of cells and the spatial relationship between the various cells in the tissue sample. According to one aspect, pathologists gather and interpret contextual information as they examine a tissue sample microscopically or through an image using the cellular structures, morphological features of various cells and tissues, or through the interpretation of a stain (i.e., immunohistochemistry (IHC) antibody stain) within the sample. The contextual information can include classification of a cell or region as tumor or normal tissue. Contextual information can also include an observation of spatial tumor heterogeneity or the recognition of an invasive margin or other biological features evident through visual inspection. Contextual information can include identification of tissue features such as the vasculature, or of immune cell infiltration. As used herein, it is understood that contextual information is destroyed in some way when a sample is lysed or ground so as to create a solution from the sample. The creation of this solution takes the various components from their biological locations and places them in a solution where at least some of the spatial relationships are lost. Contextual information can be preserved when the chemical or molecular diagnostic information can be related back to an image or sample location. In another example, chemical or molecular imaging of a sample can be considered as preserving the contextual information of a sample.

“Detect” refers to the ability to determine if an agent (such as a signal or particular antigen, protein or nucleic acid) is present or absent, for example, in a sample. In some examples, this can further include quantification, and/or localization, for example localization within a cell or particular cellular compartment. “Detecting” refers to any method of determining if something exists, or does not exist, such as determining if a target molecule is present in a biological sample. For example, “detecting” can include using a visual or a mechanical device to determine if a sample displays a specific characteristic. In certain examples, light microscopy and other microscopic means are used to detect a detectable label bound to or proximally to a target. Detecting can also encompass performing sequencing, array analysis and/or PCR amplification of a detection tag.

“Detectable Label” refers to a molecule or material that can be detected or that can produce a detectable (such as visually, electronically or otherwise) signal that indicates the presence, number, location, and/or concentration of a target, such as a target molecule, in a sample, such as a tissue sample. When conjugated to a molecule capable of binding directly or proximally to a target, the detectable label can be used to locate and/or quantify the target. A detectable label can be detected directly or indirectly, and several different detectable labels can be used in combination to detect one or more targets.

“Hybridization” refers to forming base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. The presence of a chemical which decreases hybridization (such as formamide) in the hybridization buffer will also determine the stringency (Sadhu et al, J. Biosci 6:817-821, 1984). Calculations regarding hybridization conditions for attaining particular degrees of stringency are discussed in Sambrook et at, (1989) Molecular Cloning, second edition, Cold Spring Harbor Laboratory, Plainview, N.Y. (chapters 9 and 11). Hybridization conditions for ISH are also discussed in Landegent et at, Hum. Genet. 77:366-370, 1987; Lichter et at, Hum. Genet. 80:224-234, 1988; and Pinkel et at, Proc. Natl. Acad. Sci. USA 85:9138-9142, 1988.

“Multiplex, -ed, -ing” refers to detecting multiple targets in a sample, or multiple targets in multiple samples, concurrently, substantially simultaneously, or sequentially. Multiplexing can include identifying and/or quantifying multiple distinct nucleic acids (e.g., DNA, RNA, mRNA, miRNA) and polypeptides (e.g., proteins), both individually and in any and all combinations.

“Oligonucleotide” refers to a plurality of joined nucleotides joined by phosphodiester bonds. While the number of nucleotides can vary, oligonucleotides typically include from about 6 to about 500 nucleotides. “Oligonucleotide” refers to DNA oligonucleotides, RNA oligonucleotides, synthetic oligonucleotides (e.g., sequences that are, or contain, non-naturally occurring DNA or RNA sequences), and oligonucleotide analogs, which are moieties that function similarly to oligonucleotides but which have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid molecules.

“Proximal” refers to being situated at or near a reference point. As used herein, proximal means within about 5000 nm, within about 2500 nm, within about 1000 nm, within about 500 nm, within about 250 nm, within about 100 nm, within about 50 nm, within about 10 nm, or within about 5 nm of the reference point.

“Sample” refers to a biological specimen containing biological molecules, such as genomic DNA, RNA (including mRNA), genes, amino acids, peptides, proteins, or combinations thereof, obtained from a subject. Examples include, but are not limited to, blood (e.g., peripheral blood), urine, saliva, tissue biopsy, surgical specimen, amniocentesis samples and autopsy material.

“Specific binding moiety” refers to a moiety/compound/sequence that specifically binds to a second member of a specific binding pair or a target to the substantial exclusion of binding to other potential binding pairs or non-targeted areas of a sample. Specific binding pairs are pairs of molecules that bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Exemplary specific binding moieties include, but are not limited to, proteins (e.g., antibodies or a binding variable region thereof), polynucleotides (e.g., DNA, RNA, etc.), nucleotides, oligonucleotides, polypeptides, peptides, amino acids, aptamers, a member of a specific binding pair, a primer, a plasmid, or combinations thereof.

“Target nucleic acid sequence or molecule” refers to a defined region or particular portion of a nucleic acid molecule, for example a portion of a genome (such as a gene or a region of mammalian genomic DNA containing a gene of interest). In an example where the target nucleic acid sequence is a target genomic sequence, such a target can be defined by its position on a chromosome (e.g., in a normal cell), for example, according to cytogenetic nomenclature by reference to a particular location on a chromosome; by reference to its location on a genetic map; by reference to a hypothetical or assembled contig; by its specific sequence or function; by its gene or protein name; or by any other means that uniquely identifies it from among other genetic sequences of a genome. In some examples, the target nucleic acid sequence is mammalian genomic sequence (for example human genomic sequence).

In some examples, alterations of a target nucleic acid sequence (e.g., genomic nucleic acid sequence) are “associated with” a disease or condition. In some examples, detection of the target nucleic acid sequence can be used to infer the status of a sample with respect to the disease or condition. For example, the target nucleic acid sequence can exist in two (or more) distinguishable forms, such that a first form correlates with absence of a disease or condition and a second (or different) form correlates with the presence of the disease or condition. The two different forms can be qualitatively distinguishable, such as by polynucleotide polymorphisms, and/or the two different forms can be quantitatively distinguishable, such as by the number of copies of the target nucleic acid sequence that are present in a cell.

“Unique sequence identifier” refers to a nucleic acid sequence that is configured to have a sequence that is specifically correlated to a particular target and/or specific binding moiety of a probe. Each unique sequence identifier can have a sequence that differs from other unique sequence identifiers present in a sample and can therefore be used to identify a particular target and or specific binding moiety of a probe.

### II. Overview

The present disclosure concerns various analytical techniques for biological samples, such as tissue and cell samples. The various analytical techniques contemplated by the present disclosure can include, but are not limited to, target detection, genetic sequencing (e.g., next generation sequencing), multiplexing, amplification (e.g., polymerase chain reaction, ligase chain reaction, etc.), mass spectrometry-based protein analysis, and the like. The analytical techniques disclosed herein can be facilitated by using photochemical-based technology.

The present disclosure also concerns analytical techniques and isolation techniques that address a current need in the art to provide efficient methods for analyzing multiple targets, and in some situations, a large number of targets. Analytical and isolation techniques also are disclosed that do not destroy the sample undergoing analysis, which thereby maintains sample context integrity. For example, methods for detecting targets in a tissue sample that are currently known in the art are limited to detecting only a few targets, given the types of probes available for tissue analysis. These methods also typically require various cumbersome and tissue-destructive isolation techniques (e.g., tissue lysis or laser capture microdissection) to provide isolated targets that undergo subsequent analysis. The present disclosure addresses these problems and limitations by providing probes, methods of using the probes, and selective isolation techniques exemplified by the disclosure provided herein.

### III. Probes

Disclosed herein are probe embodiments for detecting one or more targets in a sample and/or providing genetic information useful for determining the presence of genetic aberrations. In some embodiments, a probe is used to facilitate analyzing formalin-fixed paraffin embedded (FFPE) samples, fresh samples, or frozen samples. The samples may be tissue samples, cell samples, or soluble protein samples. Additionally, disclosed probes can provide substantially unlimited numbers of combinations of detection tags. This unique feature is particularly suitable for detecting multiple targets in multiplexing assays.

Particular disclosed probe embodiments comprise a photo-cleavable moiety which confers particular benefits for analytical methods and isolation. For example, using disclosed photo-cleavable moieties may reduce background signals during analysis by decreasing non-specific binding event detection. For example, duplexes formed between a template sequence and a tag sequence attached to a specifically bound probe comprising a photo-cleavable moiety can be cleaved by an irradiation event and can be subsequently isolated and analyzed. In contrast, non-specifically bound template sequences (which contribute to background noise) that hybridize with sample through a non-photo-labile interaction (e.g., electrostatic and/or hydrophobic interactions) may not be cleaved by the irradiation step and therefore can potentially be sequestered on the sample rather than isolated with the desired probes that were cleaved during the irradiation event. Under this scenario, only the desired probes (or desired portions of the probe) undergo subsequent analysis (e.g., amplification, genetic sequencing, array analysis, etc.). As such, non-photo-labile probes can be added to the sample to block sample sequences that are creating background issues. While certain probes described herein include a covalently bound photo-cleavable or photo-reactive moiety, the term probe is not so restricted. In other embodiments, the probe may include a photo-reactive moiety or a photo-cleavable moiety present on a separate molecule that interacts with the specific binding moiety and/or labeling components of the probe subsequent to irradiation.

### A. Probe Components

Certain embodiments of the probe comprise a specific binding moiety (SBM) suitable for binding to a particular binding pair, such as a biological target. The probe also may comprise a photo-reactive moiety (PRM). In illustrative embodiments, the photo-reactive moiety is a photo-cleavable moiety. In particular, the photo-cleavable moiety may be capable of being cleaved from other probe components, such as the specific binding moiety, upon exposure to light having a suitable wavelength. Embodiments of the probe may further comprise a detection tag (TAG) suitable for downstream analysis (e.g., array-based analysis, sequencing, and/or PCR amplification). Each of these components, as well as other probe components, is discussed in more detail below.

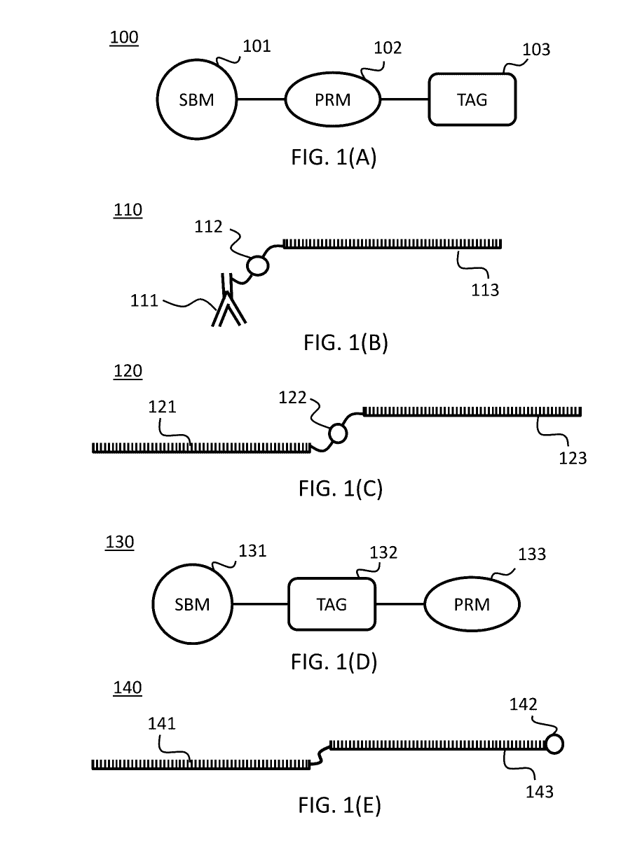

Exemplary probe embodiments are illustrated in FIGS. 1(A)-(E). FIG. 1(A) provides a generic illustration of a probe embodiment 100 comprising specific binding moiety 101, photo-cleavable moiety 102, and detection tag 103. FIG. 1(B) illustrates exemplary probe 110, wherein the specific binding moiety 101 is an antibody 111; the probe further comprising photo-cleavable moiety 102 and unique sequence identifier 113, which acts as the detection tag. Unique sequence identifier 113 may include a particular sequence, or can be configured to have a particular size (e.g., certain number of nucleotides). These variable and selectable characteristics provide the ability to uniquely and discriminately detect each different unique sequence identifier effectively coupled to a sample target by a probe. FIG. 1(C) illustrates exemplary probe 120, wherein the specific binding moiety is an oligonucleotide 121; the probe further comprising photo-cleavable moiety 122 and unique sequence identifier 123.

FIG. 1(D) provides a generic illustration of a probe embodiment 130 comprising specific binding moiety 131, detection tag 132, and photo-cleavable moiety 133. FIG. 1(E) illustrates exemplary probe 140, wherein the specific binding moiety is an oligonucleotide 141; the probe further comprising unique sequence identifier 143 and photo-cleavable moiety 142. While not shown, a probe analogous to exemplary probe 140 and 110 is understood to be within the scope of the present disclosure. In particular, another exemplary probe would include an antibody specific binding moiety with a coupling pattern analogous to 130.

### 1. Photo-Reactive Moiety (PRM)

The photo-reactive moiety is a moiety that reacts or is cleaved by light. In some embodiments, the photo-reactive moiety is cleaved from one or more probe components. In some embodiments, a photo-cleavable moiety is capable of being chemically activated by light and being separated, typically chemically separated (such as by bond cleavage), from the component (or components) to which it is coupled. In some embodiments, the cleavage event separates portions of the probe (e.g., separates the specific binding moiety from the detection tag). In other embodiments, the cleavage event cleaves a protecting group. In some embodiments, the photo-cleavable moiety is capable of absorbing light of a certain wavelength. In particular disclosed embodiments, the photo-cleavable moiety may be cleaved by ultraviolet light and/or visible light. Suitable ultraviolet light typically has a wavelength ranging from about 200 nm to about 400 nm, such as from about 250 nm to about 375 nm, or from about 260 nm to about 365 nm, or from about 280 nm to about 350 nm. Suitable visible light typically has a wavelength ranging from about 400 nm to about 790 nm, such as from about 450 nm to about 750 nm, or from about 500 nm to about 725 nm, or from about 550 nm to about 700 nm.

The photo-cleavable moiety used in certain probe embodiments may be selected based on considering certain physical properties of the probe and conditions associated with using the probe in a biological assay. For example, the photo-cleavable moiety may be selected based on the kinetics of the photo-based cleavage reaction. Thus, certain exemplary criteria for selection include the rate at which the photo-cleavable moiety is photochemically cleaved, the efficiency of photochemical cleavage, its size, and its stability under the analytical/storage conditions selected. In some embodiments, the photo-cleavable moiety should be photochemically cleaved substantially immediately upon exposure to light of the appropriate wavelength, such as at within 1 minute of being exposed to light of the appropriate wavelength, and preferably no more than about to about 5 minutes after being exposed to light of the appropriate wavelength. Preferred embodiments concern using a photo-cleavable moiety that can be cleaved under 1 minute after being exposed to light of the appropriate wavelength. In some embodiments, substantially all photo-cleavable moieties exposed to light should be cleaved within this time frame. For example, from about 50% to about 100% of the photo-cleavable moieties exposed to light of the appropriate wavelength should be cleaved, with some embodiments having from about 60% to about 99% cleavage, or from about 70% to about 95% cleavage, or from about 80% to about 90% cleavage.

The probe comprising the photo-cleavable moiety should be sufficiently stable to conditions associated with a particular analytical technique to substantially preclude degradation. For example, the probe comprising the photo-cleavable moiety should be substantially non-degraded by process solutions used, pH conditions used, and/or temperatures associated with a particular technique. Furthermore, the probe and the photo-cleavable moiety should be sufficiently robust before and after cleavage to prevent any destructive by-products from interfering with sample analysis.

In some disclosed embodiments, the photo-cleavable moiety can comprise a linker suitable for linking it with probe components disclosed herein. Suitable linkers include, but are not limited to aliphatic linkers, alkylene oxide linkers, disulfide linkers, commercially available linkers, polymeric linkers, and the like. The linker may be selected to impart increased hydrophilicity or hydrophobicity to the probe. Solely by way of example, alkylene oxide linkers disclosed herein can be used to link the photo-cleavable moiety to one or more of the probe components disclosed herein and to increase the hydrophilic nature of the entire probe.

Some embodiments of the disclosed photo-cleavable moiety have a Formula I or Formula II, provided below.

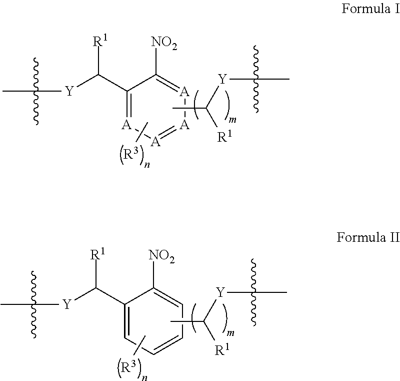

With reference to Formula I or Formula II, each R¹ independently may be halogen, hydrogen or alkyl (e.g., methyl, ethyl, propyl, and the like); each R³ independently may be selected from alkyl, aryl, hydrogen, hydroxyl, alkoxy, ether, ester, amine, amide, or halogen; each Y independently can be selected from oxygen, sulfur, NH, NR⁴ (where R⁴ is aliphatic, particularly alkyl), —C(O)—, —OC(O)—, —NHC(O)—, or halogen (e.g., CI, Br, FI, or I); each A independently may be carbon or nitrogen; n can be 0, 1, 2, or 3; and m can be 0 or 1.

Exemplary species of the photo-cleavable moiety are illustrated below. With reference to these species and the general formulas provided herein, a “ [symbol] ” symbol is used to indicate a truncated bond to other components to which the photo-cleavable moiety may be bound (e.g., a specific binding moiety, a linker, a detection tag, etc.).

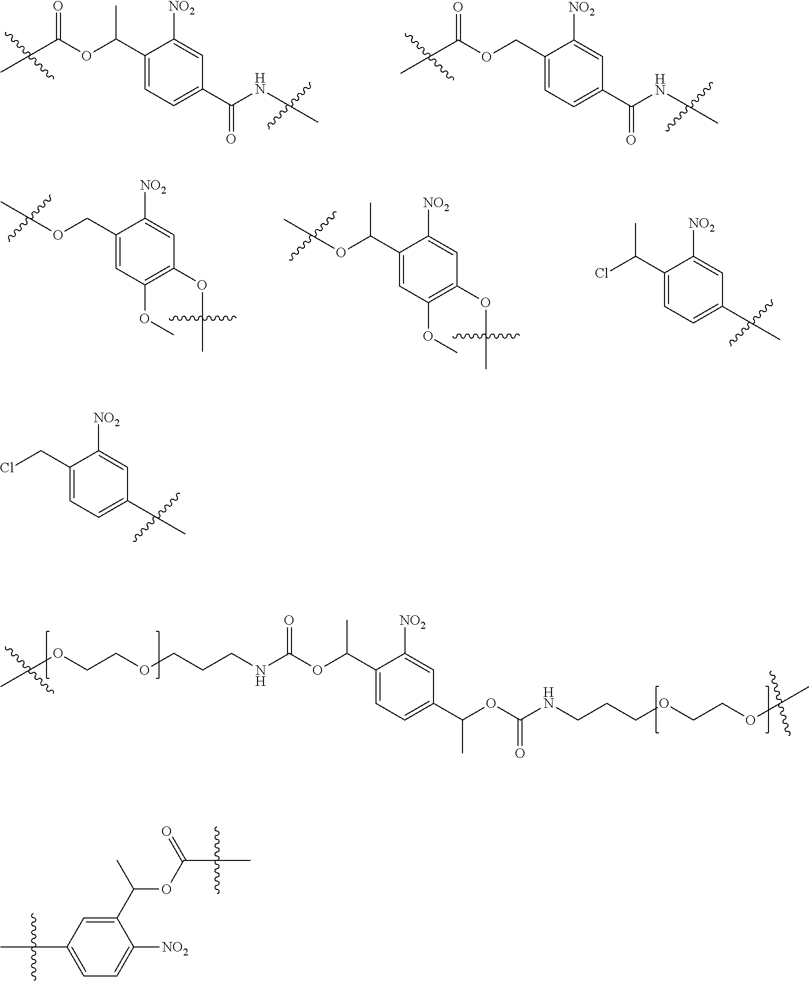

In some embodiments, the photo-cleavable moiety may have Formula III or Formula IV, provided below.

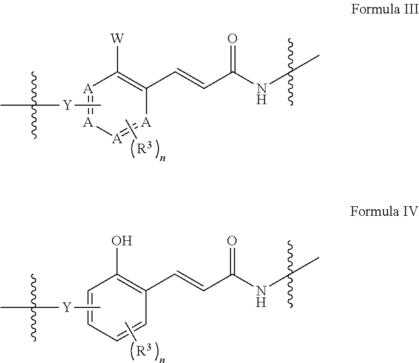

With reference to both Formula III and Formula IV, each of R³, Y, and n may be as indicated above for Formula I. The “W” variable of Formula III can be selected from OH or N(R⁴)₂ (where at least one of R⁴ is hydrogen and the other R⁴ moiety may be hydrogen or aliphatic, particularly alkyl). Exemplary embodiments of a photo-cleavable linker having a Formula II are provided below. With reference to these examples, PG refers to an amine protecting group, which would be readily recognized by persons of ordinary skill in the art.

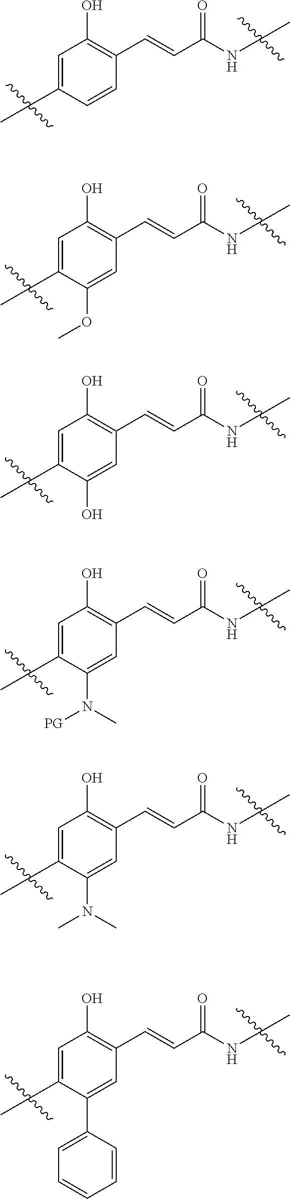

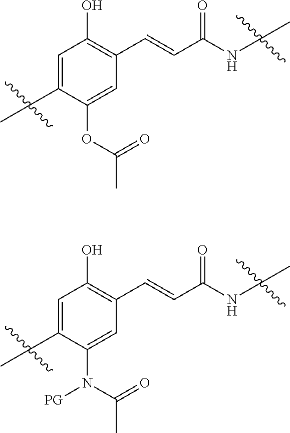

The photo-cleavable moiety may include or be bound to, either directly or indirectly, additional functional groups or moieties suitable for incorporating and/or facilitating the use of the photo-cleavable moiety in the methods disclosed herein. For example, the photocleavable moiety may be bound to a phosphoramidite to facilitate incorporation of the photo-cleavable moiety into a nucleic acid specific binding moiety. In other embodiments, the photocleavable moiety may be bound to a detectable label, such as a biotin molecule. In particular disclosed embodiments, the photo-cleavable moiety is selected from a phosphoramidite-containing photo-cleavable moiety, a biotin-containing photo-cleavable moiety, and/or an amine-containing photo-cleavable moiety. Exemplary embodiments comprising a photo-cleavable moiety are provided below.

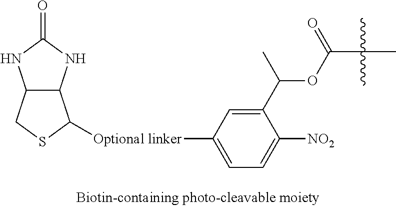

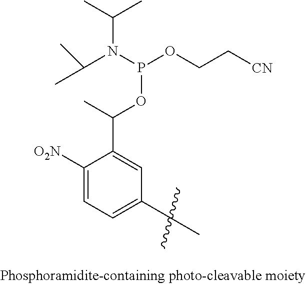 [SMILES: *c1ccc([N+](=O)[O-])c(C(C)OP(OCCC#N)N(C(C)C)C(C)C)c1]

In some embodiments, the photo-cleavable moiety may act as a linker to couple two different components together. For certain exemplary probe embodiments, the photo-cleavable moiety couples a specific binding moiety to another probe component, such as a detection tag. In other disclosed embodiments, such as when the specific binding moiety is a primer sequence, the photo-cleavable moiety may serve as a polymerase blocking moiety on the 3′ end of the primer sequence, the 5′ end of the primer sequence, or both; but more typically the 3′ end of the primer sequence. The photo-cleavable moiety can thereby block the 3′ end of the primer sequence from being extended by a polymerase enzyme on those particular portions of the sample that are not exposed to light of a suitable wavelength. Only those primers present on the irradiated portion of the sample are extended by the polymerase enzyme as the blocking portion is removed and the primer is therefore capable of being chemically modified by a polymerase enzyme. In another embodiment, caged thymidines can be used analogously. It was observed that caged thymidines can be used to inhibit in situ polymerase extension by interfering with base-pairing.

Photo-cleavable moieties also may be present on a component other than the probe. For example, particular disclosed embodiments of the method use photo-cleavable moieties to block, or cage, ATP molecules—that is, the photo-cleavable moiety prevents ATP from being used in an ATP-dependent enzymatic reaction until a sample is irradiated with light of a suitable wavelength to cleave the photo-cleavable moiety and release the ATP molecule. An exemplary blocked ATP compound (100) is provided below in Scheme 1. Upon exposure to light of a suitable wavelength, ATP compound 102 is obtained.

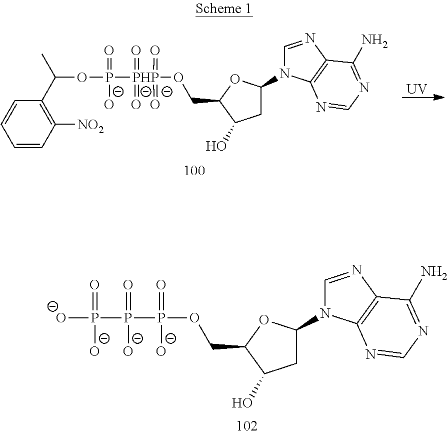

Reference is made to U.S. Pat. No. 7,432,368 and U.S. Patent Publication No. 2014/0051605, which is incorporated by reference herein in its entirety for disclosure related to methods and compositions for photo-cleavable moieties.

Similarly, in another embodiment caged deoxynucleoside triphosphates are employed to prevent extension of a primer hybridized to a target nucleic acid sequence by a polymerase enzyme (e.g., DNA polymerase and its variants, when the target is DNA, or reverse transcriptase when the target is RNA). All four deoxynucleoside triphposphates may be caged or as few as a single nucleoside trip may be caged with the remaining deoxynucleoside triphosphases being uncaged to prevent significant primer extension in the absence of illumination. An example of a caged deoxynucleoside triphosphate is caged dATP, an example of which is pictured in scheme 1 except that a deoxyribose sugar would replace the ribose sugar moiety. An example of synthesizing caged dATP is described in The Development of Methods for the Time-Resolved Imaging of the Replication of Single DNA Molecules, Richard David Perrins Ph.D. thesis, the University of Birmingham, August 2005. The preparation of caged dideoxyribosyithymine triphosphate, dideoxyadenosine triphosphate and arabinosylcytosine triphosphate is described by Meldrum et al. in “Kinetics and mechanism of DNA repair, Biochem. J. (1990) 266, 885-890. Walker et al describe “Photolabile 1-(2-Nitrophenyl)ethyl Phosphate Esters of Adenine Nucleotide Analogues. Synthesis and Mechanism of Photolysis” in J. Am. Chem. Soc. 1988, 110, 7170-7177. In another reference, Meldrum et al. also describe the “Use of Caged Compounds in Studies of the Kinetics of DNA Repair” in METHODS IN ENZYMOLOGY, VOL. 291, 1998.

### 2. Specific Binding Moieties

Specific binding moieties can be selected from an amino acid, a peptide, a protein (e.g., an antibody or a fragment thereof), a nucleic acid, a nucleotide, an oligonucleotide, a polynucleotide (e.g., DNA, RNA, miRNA, etc.), a member of a specific binding pair (e.g., biotin, avidin, streptavidin, etc.), a primer, an aptamer, a plasmid, or combinations thereof. Suitable antibodies for use as specific binding moieties include, but are not limited to, primary antibodies and/or secondary antibodies. For example, the antibody may be a primary antibody that binds directly to a target in a sample, or it may be a secondary antibody (e.g., anti-species antibody) that binds indirectly to a target in a sample through another antibody. The antibody also can be a monoclonal antibody. Suitable exemplary specific antibodies include, but are not limited to, a HER-2/neu rabbit monoclonal antibody, a PR rabbit monoclonal antibody, a topoisomerase IIa rabbit monoclonal antibody, an ER rabbit monoclonal antibody, a Ki-67 rabbit monoclonal antibody, a p53 antibody, and any other antibody that may be suitable for use in analyzing a sample having known or hereafter determined targets, including any of the particular target sequences disclosed herein. The probes disclosed herein are not limited to whole antibodies as specific binding moieties and may comprise an antibody fragment, such as a binding variable region.

Suitable oligo/polynucleotides for use as specific binding moieties include, but are not limited to, those suitable for hybridizing with a particular target sequence (or target sequence fragment). In particular disclosed embodiments, the specific binding moiety may be an oligo/polynucleotide suitable for complimentary binding with DNA or RNA. In other disclosed embodiments, the specific binding moiety may be a primer, such as a DNA primer or RNA primer that comprises at least one end suitable for extension via polymerase-mediated extension. For example, the primer may comprise a free hydroxyl group which allows extension of the primer using a polymerase enzyme. Other suitable specific binding moieties include aptamers and/or plasmids.

Members of a specific binding pair suitable for practicing the disclosed embodiments include, but are not limited to, those members that are typically employed in immunoassays and/or hybridization procedures. Particular disclosed embodiments of the specific binding pair may include avidin, biotin, streptavidin, and the like.

Certain probe embodiments concern specific binding moieties that are oligo/polynucleotides capable of binding specifically to a target sequence, or a portion of a target sequence, or antibodies capable of binding to an epitope in a target protein. Exemplary target sequences are provided herein, with certain targets of interest being selected from BCL2 (18q21.3; e.g., GENBANK™ Accession No. NC_000018, complement, nucleotides 58941559 59137593), TOP2A (17q21-q22; e.g., GENBANK™ Accession No. NC_000017, complement, nucleotides 35798321 35827695), ERG (21q22.3; e.g., GENBANK™ Accession No. NC_000021, complement, nucleotides 38675671 38955488), PTEN (10q23.3; e.g., GENBANK™ Accession No. NC_000010, nucleotides 89613175 89716382), p53 (17p13.1; e.g., GENBANK™ Accession No. NC_000017, complement, nucleotides 7512464 7531642), EGFR gene (7p12; e.g., GENBANK™ Accession No. NC_000007, nucleotides 55054219 55242525), or any one of SEQ ID NOs: 8-19.

The oligo/polynucleotide specific binding moiety may have any sequence of nucleotides capable of complementarily binding to the sequences, or portion of the sequences, of these targets, and may comprise at least 5 nucleotides to about 5,000 nucleotides (or such as about 5 to about 4,500 nucleotides, about 10 to about 4,000 nucleotides, about 20 to about 3,500 nucleotides) that bind to the target sequence (or portion of the target sequence). The oligo/polynucleotide specific binding moiety can also comprise a photo-cleavable moiety (such as a photo-cleavable phosphoramidite as disclosed herein). These embodiments of the specific binding moiety can further comprise a unique sequence identifier comprising any one of (or more) of the following: an adapter sequence, an alignment sequence, a reagent barcode sequence, with the reagent barcode sequence comprising at least 6 to about 200 nucleotides selected to have a random sequence unique to the particular oligo/polynucleotide specific binding moiety present in the probe, and/or a subject index sequence.

Embodiments of a probe comprising an antibody specific binding moiety may have the following exemplary conjugated structures: HER (such as HER1, HER2, HER3, or HER4) rabbit monoclonal antibody—photo-cleavable moiety—unique sequence identifier; PR rabbit monoclonal antibody—photo-cleavable moiety—unique sequence identifier; TOP2a rabbit monoclonal antibody—photo-cleavable moiety—unique sequence identifier; ER rabbit monoclonal antibody—photo-cleavable moiety—unique sequence identifier; Ki-67 rabbit monoclonal antibody—photo-cleavable moiety—unique sequence identifier; p53 antibody—photo-cleavable moiety—unique sequence identifier. The unique sequence identifier may have any suitable sequence, such as one of the exemplary sequences of SEQ ID NOs: 1-3 or 21-23. In other embodiments, each unique sequence identifier provided in these conjugates may be replaced with a tag sequence.

Exemplary embodiments of an oligo/polynucleotide sequence that may be used as a specific binding moiety in the disclosed probe include a chromosome 17 alpha satellite repeat sequence, such as SEQ ID NO: 4; an ALU repetitive sequence, such as SEQ ID NO: 5, or a chromosome 7 alpha satellite repeat sequence, such as SEQ ID NO: 20.

Other suitable probes include DNP-tagged probes, such as DNP-TATTTT-DNP-TATTTT, and probes comprising primer sites, such as SEQ ID NO: 6 and/or SEQ ID NO: 7. Other probes include, but are not limited to, those comprising olio/polynucleotide sequences selected from SEQ ID NOs: 34-41. In still other embodiments, the oligonucleotide specific binding moiety can be of the type disclosed in U.S. Patent Publication No. 2011/0160076, which is incorporated by reference herein in its entirety for disclosure related to methods for producing uniquely specific nucleic acid probes. In further exemplary embodiments, probes disclosed in U.S. Ser. No. 61/645,247 or in International Publication No. WO 2013/167387, both of which are incorporated herein by reference in their entirety for disclosure related to uniquely specific probes. In still other embodiments, the oligonucleotide specific binding moiety can be of the type disclosed in U.S. Pat. No. 8,420,798 or U.S. Pat. No. 7,869,959, which are incorporated by reference herein in their entirety for disclosure related to methods for selecting and/or producing nucleic acid probes.

As a specific example, the probe may comprise a primary antibody, 4B5 (available from Ventana Medical Systems, Inc.), which is capable of recognizing an epitope in the Her2 intracellular domain, such as amino acids 1231-1250 of SEQ ID NO: 10 (GAPPSTFKGTPTAENPEYLG), in the intracellular domain. The probe further comprises a photo-cleavable moiety bound to the antibody. A unique sequence identifier having any one of the sequences described in SEQ ID NOs: 1-3 or 21-23 (or a tag sequence) also is bound to the photo-cleavable moiety.

### 3. Detection Tag

Certain disclosed embodiments of the probe comprise a detection tag. In some embodiments, the detection tag can be a unique sequence identifier. In other embodiments, the detection tag can be a tag sequence.

Each unique sequence identifier has a unique sequence of nucleotides. The sequence can be designed to provide desired information. In some embodiments, the number of nucleotides in a unique sequence identifier can range from greater than one nucleotide to at least about 100 nucleotides, more typically from about 3 to about 150 nucleotides, even more typically from about 50 to about 90 nucleotides or from about 18 to about 25 nucleotides. In one embodiment, the barcode sequences are at least 18 nucleotides, while tag sequences for NGS (index tags) are typically 4-6 nucleotides. The individual sequence of each unique sequence identifier provides the ability to readily discriminate between a potentially infinite number of unique sequence identifiers present in a sample based on the sequence and/or size of the unique sequence identifier. For example, in a sample comprising mixed cell types (e.g., human and mouse cells), the unique sequence identifiers can be used to distinguish the cell types, as human cells can be detected with a probe comprising a first specific unique sequence identifier, and the mouse cells detected with a second, completely different unique sequence identifier. The unique sequence identifier may be amplified to improve detection using techniques known to those of ordinary skill in the art, such as by using the polymerase chain reaction (PCR). The unique sequence identifiers also may be analyzed for encoded information after a desired target is detected. In some embodiments, the unique sequence identifier may be sequenced, such as by using next generation sequencing. In other embodiments, the unique sequence identifier may be identified using array hybridization, such as by using a microarray (e.g., DNA microarray).

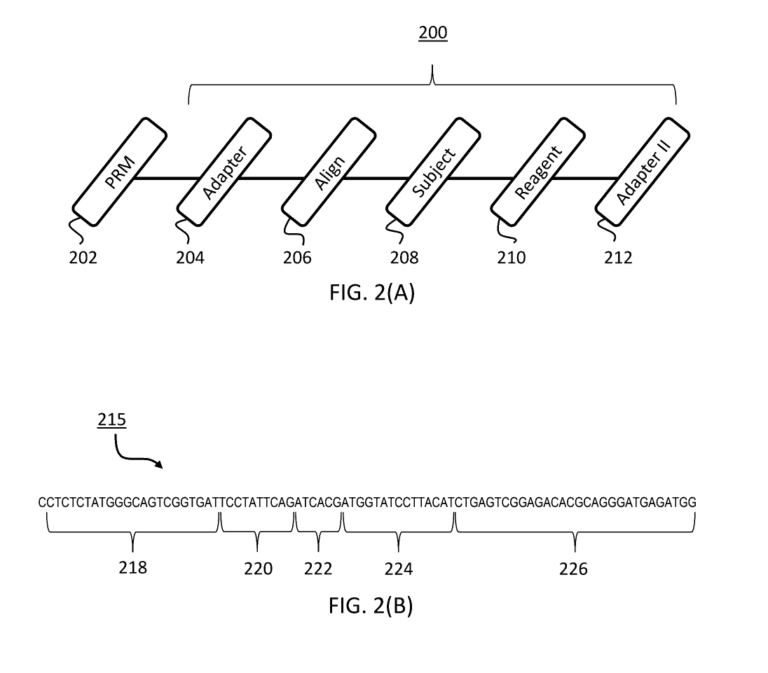

FIG. 2(A)-(B) provides (A) a schematic representation of a detection tag and (B) an illustrative unique sequence identifier contemplated by the present disclosure. Unique sequence identifier 200 can be coupled to photo-reactive moiety (PRM) 202 and comprise one or more adapter sequences (204 and 212), an alignment sequence 206, an optional patient index sequence 208, and a reagent barcode 210.

Adapter sequences 204 and/or 212 typically comprise a short nucleotide sequence that may be manipulated to facilitate analysis of the unique sequence identifier. For example, the adapter sequence may comprise a nucleotide sequence that hybridizes with a corresponding nucleic acid sequence present on a substrate used in a next-generation sequencing technique. The adapter sequence, in some embodiments, is configured for use in next generation sequencing that utilizes solid phase amplification sequencing techniques and thus uses a solid phase amplification substrate (e.g., flow cell). In other embodiments, the adapter sequence may be configured for use in emulsion PCR and thereby suitable for binding a microbead substrate. Certain disclosed adapter sequences have a sufficient number of nucleotides to hybridize with the particular next generation sequencing sample (e.g., sequences included on a solid phase amplification flow cell or sequences included on a microbead) being used, such as 1 nucleotide to at least 50 nucleotides, typically about 5 nucleotides to about 40 nucleotides, and more typically from about 15 nucleotides to about 30 nucleotides.

Unique sequence identifier 200 also may comprise one or more alignment sequences, such as sequence 206. Alignment sequences facilitate accurate sequencing by providing a starting point for reading the particular unique sequence identifier. In particular disclosed embodiments, the alignment sequence is located near the 5′ end of the unique sequence identifier. An alignment sequence comprises a sufficient number of nucleotides to allow accurate alignment for reading such as from about 3 nucleotides to at least about 20 nucleotides, more typically from about 5 to about 15 nucleotides.

Subject index sequences, such as sequence 208, have a particular unique nucleotide sequence that is designed to correlate to and thereby uniquely identify, the particular subject being analyzed. For example, a patient's identity may be equated with a subject index sequence and thus this sequence can be used to identify the unique sequence identifier isolated from a particular sample with a particular patient. In some embodiments, including a subject index sequence can be useful when multiple samples are analyzed at one time because a subject can be easily correlated with a particular sample. Subject index sequences comprise a unique nucleotide sequence comprising from about 3 to at least about 10 nucleotides; for example, 5 nucleotides, 6 nucleotides, 7 nucleotides, 8 nucleotides, 9 nucleotides, or 10 nucleotides.

Reagent barcodes, such as reagent bar code 210, provide a sequence of nucleotides that is designed to correlate to each specific binding moiety used to position the probe on targets within the sample (e.g., through hybridization, covalent bonding, antigen/antibody recognition, and the like). For example, a probe comprising an antibody as a specific binding moiety may be bound to a unique sequence identifier comprising a reagent barcode that comprises a sequence of nucleotides unique to (or that correlates to) that particular antibody. In other embodiments, the reagent barcode can be configured to encode other information, such as lot data, manufacturing data, date information, source information, or combinations thereof. In some embodiments, the reagent barcode sequence may comprise from about 6 nucleotides to about 200 nucleotides, such as about 6 nucleotides to about 150 nucleotides, or 6 nucleotides to about 100 nucleotides, or about 6 nucleotides to about 50 nucleotides.

As illustrated in FIG. 2(A), unique sequence identifier 200 may comprise an adapter sequence 204 and 212 at each end of the component. Typically, at least one adapter sequence (204 or 212) is bound to photo-reactive moiety 202. Photo-reactive moiety 202 may couple unique sequence identifier 200 to a specific binding moiety (not shown)) or photo-reactive moiety 202 may be a protecting group appending unique sequence identifier 200. Alignment sequence 206 typically is located next to adapter sequence 204 that is bound to photo-cleavable moiety 202, and reagent barcode 210 may be located directly next to, or adjacent to, alignment sequence 206. Subject index sequence 208 may be placed in between alignment sequence 206 and reagent barcode 210. Finally, second adapter sequence 212 typically is located next to the reagent barcode. Second adapter sequence 212 may facilitate linking unique sequence identifier 200 to the specific binding moiety.

A person of ordinary skill in the art will recognize that a suitable unique sequence identifier may comprise all components discussed above, or it may omit certain components. Each component can be positioned in the order illustrated by FIG. 2(A), or different component ordering may be used. For example, a person of ordinary skill in the art would recognize that the particular method of next generation sequencing used may or may not require adapter sequences. Additionally, the subject index sequence is an optional component and therefore may be present or omitted. Particular embodiments of the disclosed unique sequence identifier may be generated by a computer algorithm, which is capable of generating a substantially infinite number of unique sequence identifier sequences each having a reagent barcode comprising a different sequence of nucleotides and/or combination of adapter sequences, alignment sequences, subject index sequences, and/or reagent barcode sequences.

An exemplary unique sequence identifier 215 is illustrated in FIG. 2(B). Unique sequence identifier may be coupled to a photo-cleavable linker (not shown) at one end, which is coupled, either directly or indirectly, to an adapter sequence 218. Exemplary unique sequence identifier 215 further comprises an alignment sequence 220, a subject index sequence 222, a reagent barcode sequence 224, and a second adapter sequence 226. Other exemplary unique sequence identifiers include SEQ ID NOs: 1-3 and 21-23.

In other disclosed embodiments, the detection tag may be a tag sequence. A tag sequence, which is separate and distinct from the unique sequence identifier disclosed herein, may be used to encode information concerning a specific target on a tissue sample (e.g., probe identity, lot data, manufacturing data, date information and source information, and the like). The tag sequence also facilitates binding of a template strand to a sample that is suitable for PCR amplification. In some embodiments, the tag sequence may be bound to a protein target of the tissue sample through a specific binding moiety. Solely by way of example, a tag sequence may be bound to an antibody or an oligo/polynucleotide by a photo-cleavable linker, with the antibody or the oligo/polynucleotide serving as the specific binding moiety that binds to a particular target in the sample. The tag sequence has a sufficient number of nucleotides to facilitate hybridization with a template strand, such as from at least one nucleotide to about 25 nucleotides (such as at least one nucleotide to about 22 nucleotides, or at least one nucleotide to about 20 nucleotides, or at least one nucleotide to about 18 nucleotides).

In some embodiments, the tag sequence may comprise, or be modified to further comprise, one or more flanking primer sequences. For example, in some embodiments the tag sequence comprises a flanking primer sequence before it is bound to the sample. In other embodiments, the tag sequence may be bound to the sample and then modified with a primer sequence. In yet other embodiments, the specific binding moiety itself may be a tag sequence and can comprise, or be modified to comprise, a flanking primer sequence bound to the 5′ end of the tag sequence, the 3′ end of the tag sequence, or both the 5′ and 3′ ends of the tag sequence. Each tag sequence may include the same flanking primer sequence or different flanking primer sequences. For example, in some embodiments, the same flanking primer sequence may be used for all tag sequences within the sample. This allows simultaneous amplification of all tag sequences present within a sample.

The tag sequence may be combined with a template sequence prior to analysis in some embodiments. Template sequences, which generally comprise longer strands of nucleotides, are used to facilitate amplification techniques, such as PCR. The template sequence may be, for example, an oligonucleotide having a length suitable for quantitative PCR (e.g., an oligonucleotide having from about 50 nucleotides to about 100 nucleotides, or from about 60 nucleotides to about 90 nucleotides, or from about 70 nucleotides to about 80 nucleotides). In some embodiments, the template sequence can be configured to specifically hybridize (e.g., through sequence complementarity) with the tag sequence to form a stable duplex. A stable duplex, as disclosed herein, typically has a melting temperature (Tₘ) of greater than 60° C. In some embodiments, this duplex can be transferred into solution for further analysis via cleavage of a photo-cleavable moiety that binds the tag sequence to the specific binding moiety. Using this tag-template duplex probe avoids the need to attach one or more additional recognition biomolecules/peptide sequences directly to the template sequence, which typically is used in the art. For example, immuno-PCR methods used in the art directly conjugate a template strand to an antibody that will be bound to a target in a sample. These methods however can result in reduced antibody-antigen binding as the large template strand can often interfere with this specific binding. As disclosed herein, the template sequence may be added after the specific binding moiety is bound, which thereby reduces and preferably substantially eliminates specific binding interferences that are induced by large template sequences.

In other disclosed embodiments, the tag sequence may be modified to include one or more peptide nucleic acids and/or locked nucleic acids. A peptide nucleic acid (PNA) is an artificially synthesized polymer comprising a backbone of N-(2-aminoethyl)-glycine units linked by peptide bonds to which purine and pyrimidine bases are linked. PNAs may be used to improve hybridization with a template sequence (or a tag sequence) as PNAs do not include charged phosphate groups typically found in DNA and therefore exhibit stronger hybridization with the template sequence than would a natural DNA strand. Locked nucleic acids (LNA) comprise one or more modified nucleotides that comprise a bridge in the ribose moiety of the nucleotide that locks the ribose in the 3′-endo conformation. This conformation enhances base stacking and thereby increases the ability of the tag sequence (or a template sequence) to hybridize with the template sequence (or tag sequence). Accordingly, using either PNA or LNA in the tag sequence can reduce background signals produced by non-specific binding of template sequences and further strengthen the duplex formed between the tag sequence and the template sequence.

The disclosed detection tags are not limited in the types of nucleotides that each detection tag may comprise. For example, detection tags like unique sequence identifiers and tag sequences can comprise one or more naturally occurring nucleotides, synthesized nucleotides, chemically modified nucleotides, and combinations thereof. In some embodiments, the unique sequence identifier and/or the tag sequence can comprise one or more peptide nucleic acid or locked nucleic acid components.

The base ratio of the unique sequence identifier or tag sequence may be configured to impart chemical and/or physical modifications to the structure of the unique sequence identifier and/or tag sequence. For example, the base composition present in the tag sequence may be configured to influence the thermal melting temperature of this component upon hybridization with a template sequence. That is, the base ratio of the tag sequence may be modified to promote hybridization with a template sequence, or it may be modified to reduce tag/template hybridization. The base ratio of the template also may be modified in a similar manner. Such manipulations are not limited to the detection tag and also may be applied to specific binding moieties such as an oligo/polynucleotide specific binding moiety. These specific binding moieties can be configured to have certain base ratios that promote specific or non-specific binding with a target sequence.

The base ratio of certain probe components also can be configured to convey information about the probe. For example, the base ratio of a unique sequence identifier and/or tag sequence can be configured to have a certain base ratio that can be used to represent that particular unique sequence identifier and/or tag sequence. Once a probe comprising this particular base ratio is deposited and then isolated, the particular base ratio can be used to track the origin of that particular isolated unique sequence identifier and/or tag on the original sample.

The base ratio also may be configured to facilitate isolation of the detection tag. For example, some embodiments concern isolating a detection tag for array analysis and/or next generation sequencing. In these embodiments, the base ratio of the substrate to which the detection tag binds during the array and/or next generation sequencing analysis can be configured to specifically hybridize with the complementary base ratio present in the detection tag.

The disclosed probes also may have one or more linking facilitators that facilitate linking two or more probe components. Suitable linking facilitators include, but are not limited to, functional groups typically used in the art to promote forming one or more amide bonds, ester bonds, and the like. The linking facilitator may be a chemical compound that promotes linkage between one or more probe components, but is not incorporated into the probe. Exemplary linking facilitators include, but are not limited to, carbodiimide, NHS esters, imidoesters, maleimide, haloacetyls, pyridyldisulfides, hydrazides, alkoxyamines, diazirines, aryl azides, isocyanates, and the like.

Illustrative detection tags may be selected to be uniquely distinct tags as disclosed by U.S. Patent Publ. No. 2012/0070862, which is hereby incorporated herein by reference in its entirety for disclosure related to methods for producing uniquely distinct nucleic acid tags.

### 4. Other Components

In certain embodiments, the probe may comprise one or more additional components other than the specific binding moiety, the photo-cleavable moiety, and the detection tag.

Certain probe embodiments also may comprise a detectable label, typically coupled to a selected specific binding moiety (or moieties). Particular disclosed embodiments of the detectable label may be selected from a hapten, an enzyme, a chromophore, a fluorophore (e.g., a quantum dot), a member of a specific binding pair, or combinations thereof. These detectable labels can be used to facilitate active purification of the detection tag. Haptens useful in the disclosed method include, but are not limited to, oxazoles, pyrazoles, thiazoles, nitroaryls, benzofurans, triterpenes, ureas, thioureas, rotenoids, coumarins, cyclolignans, di-nitrophenyls, biotin, digoxigenin, fluoresceins, or rhodamines. Particular embodiments of the haptens that may be used in the presently disclosed embodiments are disclosed in U.S. Pat. No. 7,695,929, which is incorporated herein by reference in its entirety. Exemplary hapten species include, but are not limited to, 5-nitro-3-pyrazole carbamide, 2-(3,4-dimethoxyphenyl)quinoline-4-carboxylic acid), 3-hydroxy-2-quinoxalinecarbamide, 2,1,3-benzoxadiazole-5-carbamide, and 2-acetamido-4-methyl-5-thiazolesulfonamide. Other exemplary haptens include

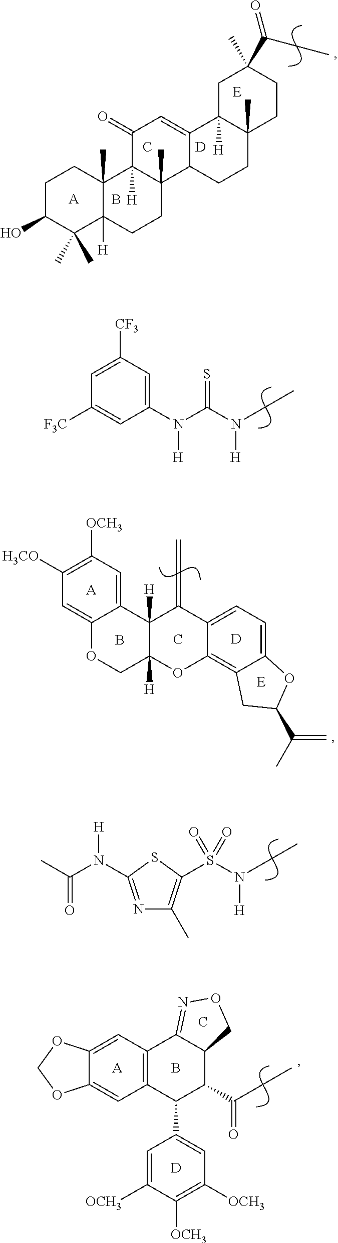

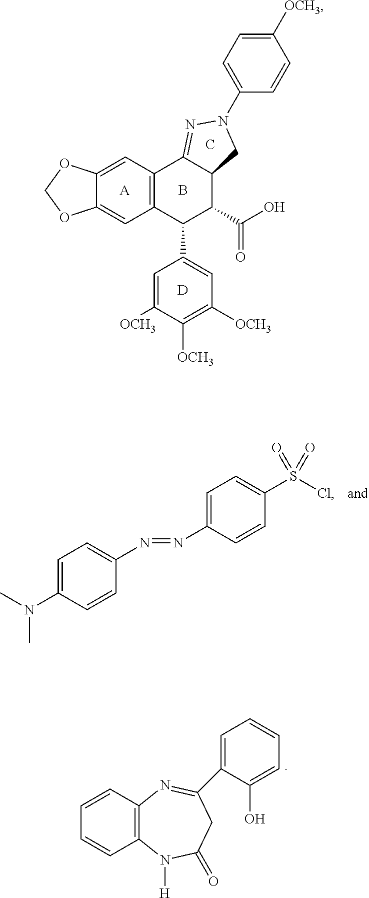

In particular disclosed embodiments, the detectable label may be coupled with a primer to facilitate isolation and/or purification in subsequent isolation steps. For example, the detectable label could be coupled to a nucleotide of the primer (such as through a functionalized base of the nucleotide, or via phosphoramidite-based oligonucleotide synthesis). Also, probes may comprise a detectable label bound to a tag sequence or unique sequence identifier to facilitate detection. Similar base-functionalization and/or phosphoramidite-based oligonucleotide synthetic methods may be used to produce these particular probes. In some embodiments the detectable label may be attached to a nucleoside triphosphate that becomes incorporated by extension of the primer sequence by a polymerase.

In some embodiments, the probe may further comprise a signal-amplification moiety, such as a tyramine or tyramine derivative, which can be used to increase the intensity of the signal generated by a detectable label. Accordingly, tyramide signal amplification (TSA) may be used with embodiments of the disclosed probe. Particular examples of suitable tyramine derivatives, as well as methods of attaching the tyramine derivatives, are disclosed in U.S. Patent Publication No. 2013/0109019, which is incorporated herein by reference. A suitable tyramine derivative may have a formula

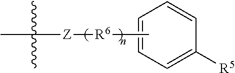

where R⁵ is selected from hydroxyl, ether, amine, and substituted amine; R⁶ is selected from alkyl, alkenyl, alkynyl, aryl, heteroaryl, —ORₘ, —NRₘ, and —SRₘ, where m is 1-20; n is 1-20; Z is selected from oxygen, sulfur, or NR^{a} wherein R^{a} is selected from hydrogen, aliphatic, aryl, or alkyl aryl. The tyramine derivative may be attached to a tag sequence and a photo-cleavable moiety through the Z moiety of the tyramine derivative. In some embodiments, the Z moiety of the tyramine derivative may be coupled with a photo-cleavable moiety, which is also coupled with a tag sequence.

An exemplary synthetic scheme is provided below in Scheme 2. Tyramide compound 200 may be combined with photo-cleavable moiety 202 to form conjugate 204. Conjugate 204 may be combined with a tag sequence having an exposed amine moiety capable of coupling with conjugate 204 to form tyramide-containing conjugate 206. A person of ordinary skill in the art will recognize that the particular order of coupling described in Scheme 2 may be modified. For example, photo-cleavable moiety 202 could be first coupled with a tag sequence and then the signal amplification compound 200.

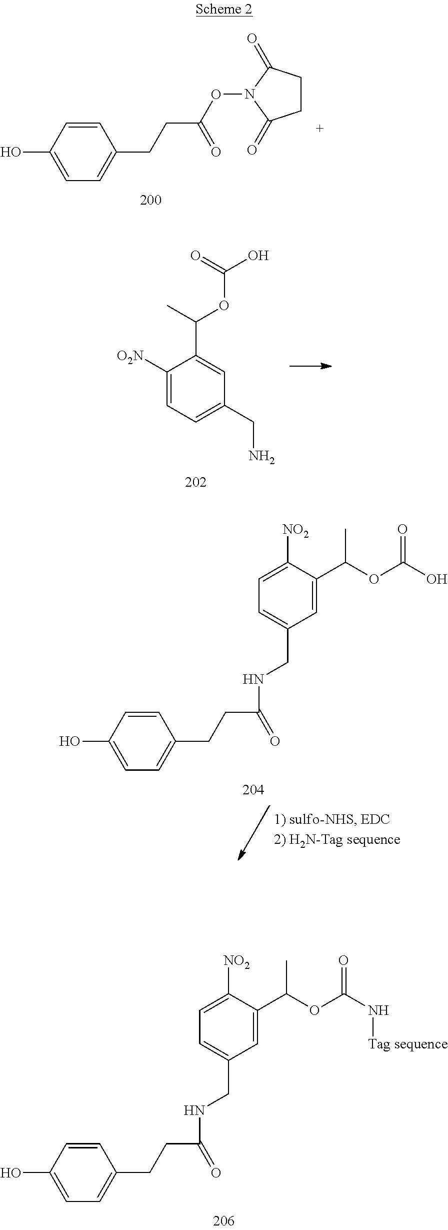

The tyramine or tyramine derivative may be used to deposit multiple tag sequences proximal to a desire target area of the sample. Adding a peroxidase, such as horseradish peroxidase, to the sample sequentially with, or simultaneously with, the tyramine-containing probe facilitates deposition of the tag sequences.

### B. Making the Probe

The probes described herein may be made by any method now known or hereafter developed. The method for making the probe may be selected based, at least in part, on the particular components being included in the probe. The methods disclosed herein may be altered and modified to accommodate different components than those expressly disclosed herein as will be understood by a person of ordinary skill in the art.

Probes comprising specific binding moieties, a photo-cleavable moiety, and a detection tag may be made using coupling conditions known in the art. For example, if the specific binding moiety is an antibody, the method may comprise coupling an antibody to the photo-cleavable moiety through a functional group of the antibody (e.g., amine, thiol, carbonyl, etc.). The photo-cleavable moiety may include (or be modified to include) a reactive functional group, such as an NHS ester, maleimide, etc. These two components can be coupled using coupling reagents typically used in the art, such as EDCI, DCC, EDC, etc. For example, Scheme 3 illustrates an embodiment wherein photo-cleavable moiety 300 is coupled with an NHS group to provide activated NHS ester 302. Ester 302 is then reacted with an antibody to provide an antibody labeled with the photo-cleavable moiety (conjugate 304). A person of ordinary skill in the art will recognize that this method can be suitably modified for any of the particular photo-cleavable moieties disclosed herein.

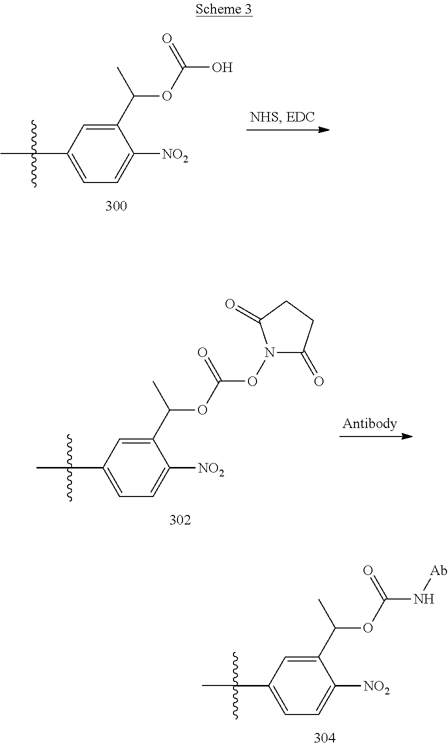

In some embodiments using an oligo/polynucleotide as the specific binding moiety, the photo-cleavable moiety may be chemically integrated into the oligo/polynucleotide by using phosphoramidite reagents comprising the photo-cleavable functional group. An oligonucleotide synthesizer may be used to integrate the photo-cleavable phosphoramidite. An exemplary embodiment is illustrated below in Scheme 4. As illustrated in Scheme 4, phosphoramidite reagent 400, which comprises a photo-cleavable moiety, is coupled to nucleotide 402 of an oligo/polynucleotide to provide labeled oligo/polynucleotide 404, which is then oxidized to corresponding phosphate 406. The cyanoester moiety is then removed to provide unprotected phosphate 408. As previously indicated, the photo-cleavable moiety may comprise other functional groups that may be further manipulated to be coupled with the tag sequence, unique sequence identifier, or other probe components disclosed herein. As illustrated in Scheme 4, and provided solely as an example, photo-cleavable moiety 400 further comprises a protected amine group —NH—PG, where PG is an amine protecting group. The protecting group may be removed to allow coupling the photo-cleavable moiety to one of more of the probe components disclosed herein.

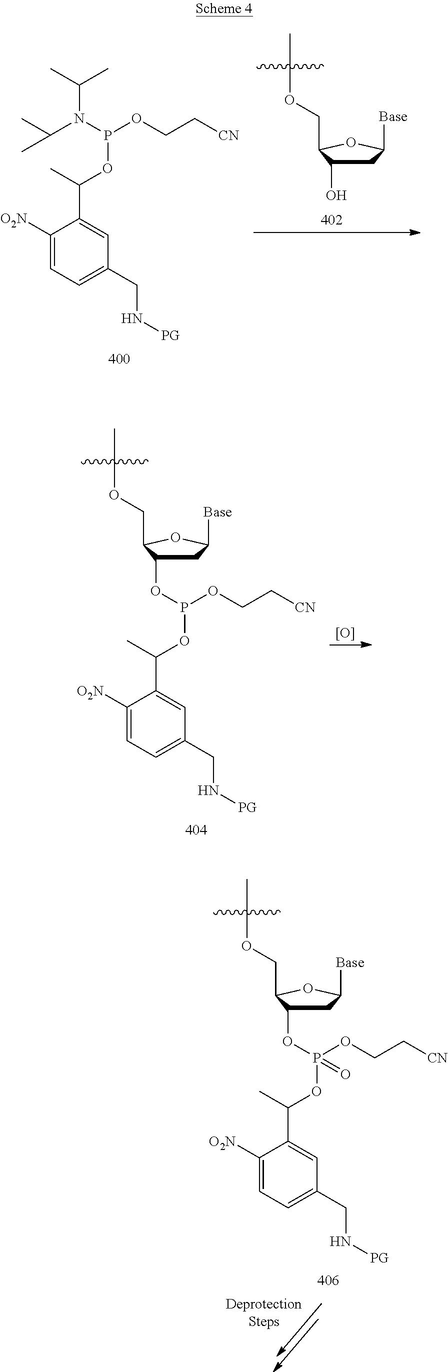

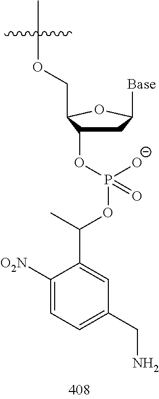

In some embodiments, the specific binding moiety may be bound to a detectable label using coupling conditions known in the art, such as Fc-specific coupling conditions, non-specific coupling conditions, and the like. In some embodiments, the detectable label may be coupled to an antibody through a thiol moiety of the antibody. In some embodiments, a detectable label may be coupled to an antibody through an aldehyde moiety generated on the antibody.

The detectable label also may be coupled to the specific binding moiety through a linker. Suitable linkers include, but are not limited to aliphatic linkers, alkylene oxide linkers, disulfide-containing linkers, commercially available linkers, polymeric linkers, and the like. One particular embodiment of a class of linkers for use with disclosed probe components is a polyalkyleneglycol linker having the general structure shown below:

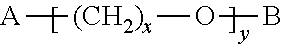

wherein A and B include similar or different reactive groups suitable for reacting with the probe components disclosed herein (e.g., carbonyl-reactive groups, amine-reactive group, thiol-reactive group, and the like, and that are suitable for reacting with a detectable label or a specific binding moiety), x is an integer from 2 to 10 (such as 2, 3 or 4), and y is an integer from 1 to 50, for example, from 2 to 30 such as from 3 to 20 or from 4 to 12. Exemplary linkers, such as PEG linkers, and methods for coupling a specific binding moiety to a detectable label using such linkers, are provided in U.S. Pat. No. 7,695,929, which is incorporated herein by reference. These disclosed linkers also may be used to couple various components of the probe other than just coupling the detectable label and the specific binding moiety. For example, a linker may be used to couple the specific binding moiety to a detection tag and/or a detectable label, or one or more linkers may be used to couple the photo-cleavable moiety with one or more of the other probe components disclosed herein. Linkers may be cleaved using any of the primary and/or secondary cleavage techniques disclosed herein.

Some embodiments of the disclosed method concern using an irradiation event that photochemically induces formation of a compound or radical species that can cleave a chemically-cleavable linker. The compound or radical species formed by the irradiation event should be capable of interacting with one or more functional groups of a linker present in the probe (or a different probe species) in a manner that causes cleavage of the linker. Solely by way of example, a probe moiety may comprise a photo-cleavable moiety that can be cleaved by an irradiation event to provide a species, such as a radical species, that can then subsequently cleave another non-photo-labile linker in the probe. In other embodiments, the light-induced cleavage event can promote cleavage of a photo-cleavable moiety that comprises one or more moieties that can be cleaved and/or released from the photo-cleavable moiety to form a separate chemical species capable of interacting with a linker species present in the probe (or a different probe).

### C. Probe Modifications

One or more of the probe components disclosed herein may be configured to be more or less robust under certain conditions. For example, a linker and/or a photo-cleavable moiety present in the probe may be photochemically and/or chemically induced to become a species that is more or less robust after such photochemical and/or chemical manipulation. In some embodiments, specifically bound probes can be configured to have a linker and/or photo-cleavable moiety that become(s) more robust before or after being bound to the sample. These probes will therefore comprise detection tags that are not cleaved upon exposure to light of a particular wavelength. Probes that do not comprise the more robust photo-cleavable moiety are cleaved. A secondary cleavage event may then be implemented to cleave the specifically bound probes, such as by using light of a different wavelength, longer time periods of irradiation, more intense light sources, thermal cleavage, chemical cleavage, enzymatic cleavage, or combinations thereof.

Such techniques also may be applied to components of the probe that are not photochemically labile, but rather are chemically labile. For example, a linker (other than a photo-cleavable moiety) present in the probe may be configured to be more robust after the probe is bound to the sample (e.g., through chemical modification of one or more functional group of the linker). The robust linker is then not susceptible to chemical cleavage under conditions where it would otherwise normally be cleaved. Manipulation and/or analysis of the sample may then be conducted using such conditions that do not chemically alter the robust linkers. The robust linker may then be subsequently cleaved (such as chemically cleaved) using conditions selected particularly for the robust linker (e.g., conditions that are more basic and/or acidic than typical physiologically desired).

In some embodiments, the linker and/or photo-cleavable moiety may be configured to be so robust as to be impervious to a cleavage event (e.g., photo-chemical and/or chemical cleavage) used for an initial analysis. Accordingly, a sample comprising probes having these robust components can be stored without degrading. Such samples can then be analyzed at a later time.

The probe also may be configured to have components that facilitate cell wall permeation. In some embodiments, the probe is specifically designed to permeate a cell wall to facilitate analysis of cell components (e.g., the nucleus, mitochondria, ribosomes, and the like). Such probes can be configured to comprise one or more components that promote lipid-solubility of the probe (e.g., aliphatic linkers, aliphatic functional groups on a photo-cleavable moiety or other probe component).

In other disclosed embodiments, the hydrophobicity or hydrophilicity of the probe can be configured to attain solubility (or insolubility) in certain process solutions used for sample analysis. For example, the probe can be configured to be more or less hydrophilic, and therefore more or less soluble in developing solutions used with photoresist materials. A person of ordinary skill in the art will recognize that the type of developing solution that is being used will govern whether the probe should be configured to be more or less hydrophobic (or hydrophilic).

The hydrophobicity and/or hydrophilicity of the probe can also be configured to promote solubility in process solutions, such as the elution solutions used to isolate detection tags after a cleavage event. In these embodiments, if an aqueous buffer is used as the elution solution, the portion of the probe that will end up being cleaved (e.g., detection tag) can be configured to be hydrophilic so as to promote its elution from the sample. If the cleaved portion is not to be isolated, the probe can be configured (prior to or after being added to the sample) to be less hydrophilic so that it does not elute with aqueous elution buffers.

### IV. Targets

Embodiments of the probe and method disclosed herein may be used to identify and/or quantify many different biological targets. Throughout this disclosure when reference is made to a target protein, it is understood that any polynucleotides associated with that protein can also be used as a target. The target may be a protein or nucleic acid molecule from a pathogen, such as a virus, bacteria, or intracellular parasite, such as from a viral genome. For example, a target protein may be produced from a target polynucleotide associated with (e.g., correlated with, causally implicated in, etc.) a disease. In certain disclosed embodiments, the target (or targets) of interest may be a particular nucleic acid sequence that may comprise a genetic aberration, such as a single nucleotide polymorphism, promoter methylation, mRNA expression, siRNA, a particular copy number change, a mutation, a certain expression level, a rearrangement, or combination thereof. In some embodiments, the targets are soluble proteins obtained from biological samples, such as serum, plasma, and/or urine. Some embodiments of the disclosed method may be used to detect and quantify DNA, RNA, and proteins of the same target (e.g., HER2) simultaneously from the same sample (e.g., from the same tissue section).

The disclosed method may be used to detect microRNA (miRNA or miR). MicroRNAs are small, non-coding RNAs that negatively regulate gene expression, such as by translation repression. For example, miR-205 regulates epithelial to mesenchymal transition (EMT), a process that facilitates tissue remodeling during embryonic development. However, EMT also is an early step in tumor metastasis. Down-regulation of microRNAs, such as miR-205, may be an important step in tumor progression. For instance, expression of miR-205 is down-regulated or lost in some breast cancers. MiR-205 also can be used to stratify squamous cell and non-small cell lung carcinomas (J. Clin. Oncol., 2009, 27(12):2030-7). Other microRNAs have been found to modulate angiogenic signaling cascades. Down-regulation of miR-126, for instance, may exacerbate cancer progression through angiogenesis and increased inflammation. Thus, microRNA expression levels may be indicative of a disease state.

A target nucleic acid sequence can vary substantially in size. Without limitation, the nucleic acid sequence can have a variable number of nucleic acid residues. For example a target nucleic acid sequence can have at least about 10 nucleic acid residues, or at least about 20, 30, 50, 100, 150, 500, 1000 residues. Similarly, a target polypeptide can vary substantially in size. The target polypeptides typically include at least one epitope that binds to a peptide specific antibody, or fragment thereof. In some embodiments that polypeptide can include at least two epitopes that bind to a peptide specific antibody, or fragment thereof.

In specific, non-limiting examples, a target protein is produced by a target nucleic acid sequence (e.g., genomic target nucleic acid sequence) associated with a neoplasm (for example, a cancer). Numerous chromosome abnormalities (including translocations and other rearrangements, amplification or deletion) have been identified in neoplastic cells, especially in cancer cells, such as B cell and T cell leukemias, lymphomas, breast cancer, colon cancer, neurological cancers and the like. In some examples, therefore, at least a portion of the target molecule is produced by a nucleic acid sequence (e.g., genomic target nucleic acid sequence) amplified or deleted in at least a subset of cells in a sample. In one example, the genomic target nucleic acid sequence is selected to include a gene (e.g., an oncogene) that is reduplicated in one or more malignancies (e.g., a human malignancy). Oncogenes are known to be responsible for several human malignancies. For example, chromosomal rearrangements involving the SYT gene located in the breakpoint region of chromosome 18q11.2 are common among synovial sarcoma soft tissue tumors.

For example, HER2, also known as c-erbB2 or HER2/neu, is a gene that plays a role in the regulation of cell growth (a representative human HER2 genomic sequence is provided at GENBANK™ Accession No. NC_000017, nucleotides 35097919-35138441). The gene codes for a 185 kd transmembrane cell surface receptor that is a member of the tyrosine kinase family. HER2 is amplified in human breast, ovarian, and other cancers; therefore, a HER2 gene (or a region of chromosome 17 that includes the HER2 gene) can be used as a genomic target nucleic acid sequence. Other breast cancer relevant proteins include the estrogen receptor (ER) and progesterone receptor (PR).

In other examples, a target protein produced from a nucleic acid sequence (e.g., genomic target nucleic acid sequence) is selected that is a tumor suppressor gene that is deleted (lost) in malignant cells. For example, the p16 region (including D9S1749, D9S1747, p16(INK4A), p14(ARF), D9S1748, p15(INK4B), and D9S1752) located on chromosome 9p21 is deleted in certain bladder cancers. Chromosomal deletions involving the distal region of the short arm of chromosome 1 (that encompasses, for example, SHGC57243, TP73, EGFL3, ABL2, ANGPTL1, and SHGC-1322), and the pericentromeric region (e.g., 19p13-19q13) of chromosome 19 (that encompasses, for example, MAN2B1, ZNF443, ZNF44, CRX, GLTSCR2, and GLTSCR1) are characteristic molecular features of certain types of solid tumors of the central nervous system.

The aforementioned examples are provided solely for purpose of illustration and are not intended to be limiting. Numerous other cytogenetic abnormalities that correlate with neoplastic transformation and/or growth are known to those of ordinary skill in the art. Target proteins that are produced by nucleic acid sequences (e.g., genomic target nucleic acid sequences), which have been correlated with neoplastic transformation and which are useful in the disclosed methods, also include (GENBANK™ RefSeq Accession No. in parentheses) the EGFR gene (7p12; e.g., NC_000007, nucleotides 55054219-55242525), the C-MYC gene (8q24.21; e.g., NC_{—} 000008, nucleotides 128817498-128822856), D5S271 (5p15.2), lipoprotein lipase (LPL) gene (8p22; e.g., NC_000008, nucleotides 19841058-19869049), RB1 (13q14; e.g., NC_000013, nucleotides 47775912-47954023), p53 (17p13.1; e.g., NC_000017, complement, nucleotides 7512464-7531642), N-MYC (2p24; e.g., NC_000002, complement, nucleotides 151835231-151854620), CHOP (12q13; e.g., NC_000012, complement, nucleotides 56196638-56200567), FUS (16p11.2; e.g., NC_000016, nucleotides 31098954-31110601), FKHR (13p14; e.g., NC_000013, complement, nucleotides 40027817-40138734), as well as, for example: ALK (2p23; e.g., NC_{—} 000002, complement, nucleotides 29269144-29997936), Ig heavy chain, CCND1 (11q13; e.g., NC_000011, nucleotides 69165054-69178423), BCL2 (18q21.3; e.g., NC_{—} 000018, complement, nucleotides 58941559-59137593), BCL6 (3q27; e.g., NC_{—} 000003, complement, nucleotides 188921859-188946169), MALF1, AP1 (1p32-p31; e.g., NC_000001, complement, nucleotides 59019051-59022373), TOP2A (17g21-q22; e.g., NC_000017, complement, nucleotides 35798321-35827695), TMPRSS (21 q22.3; e.g., NC_000021, complement, nucleotides 41758351-41801948), ERG (21 q22.3; e.g., NC_000021, complement, nucleotides 38675671-38955488); ETV1 (7p21.3; e.g., NC_000007, complement, nucleotides 13897379-13995289), EWS (22q12.2; e.g., NC_000022, nucleotides 27994271-28026505); FLI1 (11q24.1-q24.3; e.g., NC_000011, nucleotides 128069199-128187521), PAX3 (2q35-q37; e.g., NC_{—} 000002, complement, nucleotides 222772851-222871944), PAX7 (1p36.2-p36.12; e.g., NC_000001, nucleotides 18830087-18935219), PTEN (10q23.3; e.g., NC_000010, nucleotides 89613175-89716382), AKT2 (19q13.1-q13.2; e.g., NC_000019, complement, nucleotides 45431556-45483036), MYCL1 (1p34.2; e.g., NC_000001, complement, nucleotides 40133685-40140274), REL (2p13-p12; e.g., NC_000002, nucleotides 60962256-61003682) and CSF1R (5q33-q35; e.g., NC_000005, complement, nucleotides 149413051-149473128).

In other examples, a target protein is selected from a virus or other microorganism associated with a disease or condition. Detection of the virus- or microorganism-derived target nucleic acid sequence (e.g., genomic target nucleic acid sequence) in a cell or tissue sample is indicative of the presence of the organism. For example, the target peptide, polypeptide or protein can be selected from the genome of an oncogenic or pathogenic virus, a bacterium or an intracellular parasite (such as Plasmodium falciparum and other Plasmodium species, Leishmania (sp.), Cryptosporidium parvum, Entamoeba histolytica, and Giardia lamblia, as well as Toxoplasma, Eimeria, Theileria, and Babesia species).

In some examples, the target protein is produced from a nucleic acid sequence (e.g., genomic target nucleic acid sequence) from a viral genome. Exemplary viruses and corresponding genomic sequences (GENBANK™ RefSeq Accession No. in parentheses) include human adenovirus A (NC_001460), human adenovirus B (NC_004001), human adenovirus C (NC_001405), human adenovirus D (NC_002067), human adenovirus E (NC_003266), human adenovirus F (NC_001454), human astrovirus (NC_001943), human BK polyomavirus (V01109; GI:60851) human bocavirus (NC_007455), human coronavirus 229E (NC_002645), human coronavirus HKU1 (NC_006577), human coronavirus NL63 (NC_005831), human coronavirus OC43 (NC_005147), human enterovirus A (NC_001612), human enterovirus B (NC_001472), human enterovirus C (NC_001428), human enterovirus D (NC_001430), human erythrovirus V9 (NC_004295), human foamy virus (NC_001736), human herpesvirus 1 (Herpes simplex virus type 1) (NC_001806), human herpesvirus 2 (Herpes simplex virus type 2) (NC_001798), human herpesvirus 3 (Varicella zoster virus) (NC_001348), human herpesvirus 4 type 1 (Epstein-Barr virus type 1) (NC_007605), human herpesvirus 4 type 2 (Epstein-Barr virus type 2) (NC_009334), human herpesvirus 5 strain AD169 (NC_001347), human herpesvirus 5 strain Merlin Strain (NC_006273), human herpesvirus 6A (NC_001664), human herpesvirus 6B (NC_000898), human herpesvirus 7 (NC_001716), human herpesvirus 8 type M (NC_003409), human herpesvirus 8 type P (NC_009333), human immunodeficiency virus 1 (NC_001802), human immunodeficiency virus 2 (NC_001722), human metapneumovirus (NC_004148), human papillomavirus-1 (NC_001356), human papillomavirus-18 (NC_001357), human papillomavirus-2 (NC_001352), human papillomavirus-54 (NC_001676), human papillomavirus-61 (NC_001694), human papillomavirus-cand90 (NC_004104), human papillomavirus RTRX7 (NC_004761), human papillomavirus type 10 (NC_001576), human papillomavirus type 101 (NC_008189), human papillomavirus type 103 (NC_008188), human papillomavirus type 107 (NC_009239), human papillomavirus type 16 (NC_001526), human papillomavirus type 24 (NC_001683), human papillomavirus type 26 (NC_001583), human papillomavirus type 32 (NC_001586), human papillomavirus type 34 (NC_001587), human papillomavirus type 4 (NC_001457), human papillomavirus type 41 (NC_001354), human papillomavirus type 48 (NC_001690), human papillomavirus type 49 (NC_001591), human papillomavirus type 5 (NC_001531), human papillomavirus type 50 (NC_001691), human papillomavirus type 53 (NC_001593), human papillomavirus type 60 (NC_001693), human papillomavirus type 63 (NC_001458), human papillomavirus type 6b (NC_001355), human papillomavirus type 7 (NC_001595), human papillomavirus type 71 (NC_002644), human papillomavirus type 9 (NC_001596), human papillomavirus type 92 (NC_004500), human papillomavirus type 96 (NC_005134), human parainfluenza virus 1 (NC_003461), human parainfluenza virus 2 (NC_003443), human parainfluenza virus 3 (NC_001796), human parechovirus (NC_001897), human parvovirus 4 (NC_007018), human parvovirus B19 (NC_000883), human respiratory syncytial virus (NC_001781), human rhinovirus A (NC_001617), human rhinovirus B (NC_001490), human spumaretrovirus (NC_001795), human T-lymphotropic virus 1 (NC_001436), human T-lymphotropic virus 2 (NC_001488).

In certain examples, the target protein is produced from a nucleic acid sequence (e.g., genomic target nucleic acid sequence) from an oncogenic virus, such as Epstein-Barr Virus (EBV) or a Human Papilloma Virus (HPV, e.g., HPV16, HPV18). In other examples, the target protein produced from a nucleic acid sequence (e.g., genomic target nucleic acid sequence) is from a pathogenic virus, such as a Respiratory Syncytial Virus, a Hepatitis Virus (e.g., Hepatitis C Virus), a Coronavirus (e.g., SARS virus), an Adenovirus, a Polyomavirus, a Cytomegalovirus (CMV), or a Herpes Simplex Virus (HSV).

### V. Method of Use

Disclosed herein are several embodiments of a method of using probe embodiments described herein, as well as novel methods for isolating and/or analyzing a tissue sample, or other cellular specimen, while maintaining tissue context and integrity. In particular disclosed embodiments, the method may further comprise employing subsequent analytical techniques (e.g., PCR, next generation sequencing, array hybridization, etc.) to provide improved multiplexing capabilities, increased sensitivity, relative quantification, and/or improved resolution.

### A. General Method

In particular disclosed embodiments, the method comprises providing a biological sample, such as a tissue sample, comprising one or more targets of interest. The biological sample is contacted with a probe using conditions sufficient to facilitate binding of the specific binding moiety to the target. In some embodiments, the probe may comprise a specific binding moiety, a photo-cleavable moiety, and a detection tag. In other embodiments, the probe may not comprise a photo-cleavable moiety; rather, the sample is exposed to a probe and a composition comprising a separate component comprising a photo-cleavable moiety. The method may further comprise exposing particular selected sections of the biological sample to light having a wavelength and intensity suitable to cleave the photo-cleavable moiety thereby freeing the detection tag. The detection tag may then be detected. The presence and/or location of target may be determined by detecting (e.g., visually and/or with instrumentation) a detectable label. The identity of the target also may be determined by photochemically isolating portions of the probe, such as a tag sequence, tag-template duplex, and/or a unique sequence identifier. The method may have one or more additional steps, one or more steps may be performed manually, or certain or all of the steps may be performed by an automated system.

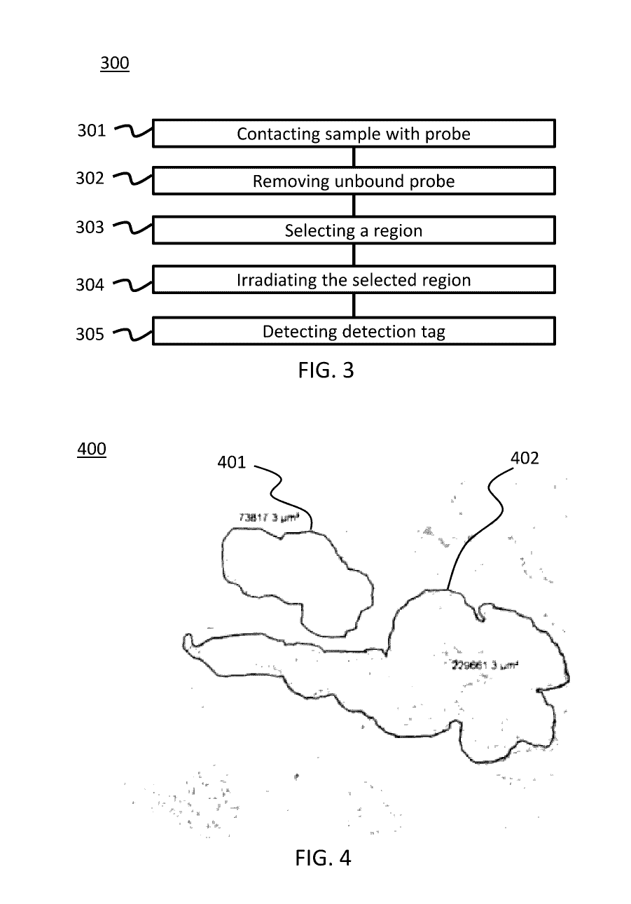

FIG. 3 is a schematic that illustrates a method for performing contextual molecular diagnostics 300 showing a step of contacting a sample with a probe 301, removing unbound probe 302, selecting a region 303, irradiating the selected region 304, and detecting the detection tag 305.

FIG. 4 is a photomicrograph showing that a first region 401 and a second region 402 can be selected. In particular, an image of the sample can be acquired and stored as a digital file. The digital file can be manipulated by a user or through the application of image analysis algorithms to select one or more regions of interest. These selected regions can then been irradiated. In various embodiments, the regions can be irradiated concurrently or sequentially. The detection tags coming from the first and second region can either be detected concurrently or sequentially.

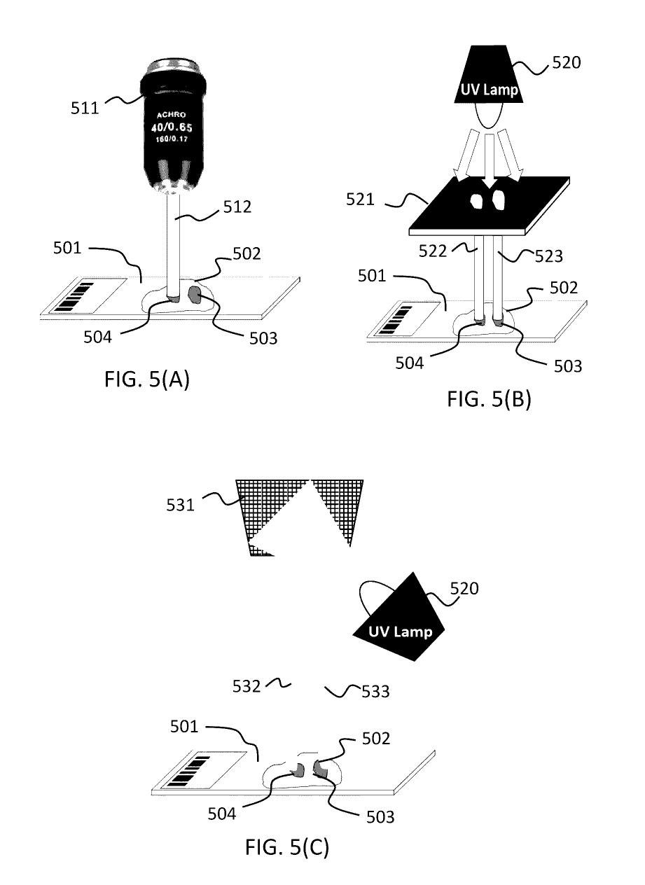

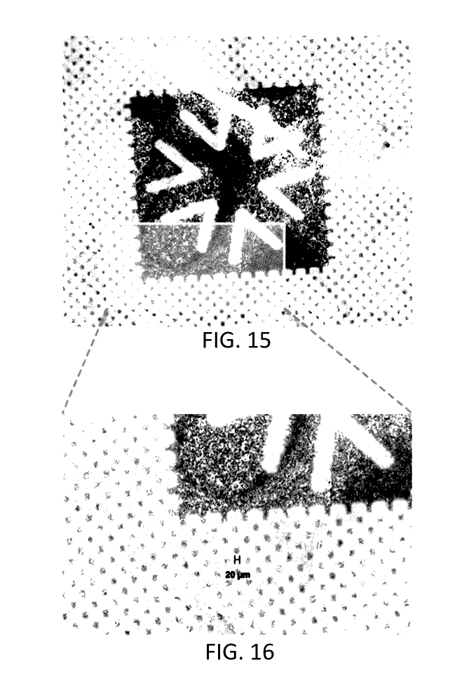

FIG. 5(A)-(C) are schematic drawings showing several approaches to irradiating a first selected region 504 and/or a second selected region 503 of a sample 502 disposed on a slide 501. Referring now to FIG. 5(A), a microscope objective 511 can be used to view the sample so that a region of interest can be selected. The microscope objective can also be used to direct light irradiating the sample. For instance, region of interest 504 may be selected in the view of objective 511. Light from an appropriate source can then be directed through objective 511 to region region 504. Another similar approach would be to combine this with raster scanning of the specimen to illuminate the entire selected region. Raster scanning would be achieved by stage translation in the X and Y directions or deflection of the light with rotating mirrors. The intensity of the light beam can be modulated (e.g., UV LED with electronic control of output intensity) as the beam position is scanned across a rectangular region of the specimen to create the illuminated region, or the beam can always be on and the raster scan is restricted to only the boundaries of the selected region. FIG. 5(C) shows a schematic depiction of a digital micromirror device (DMD) being used to selectively irradiate portions of the slide. This embodiment was used to make the elaborate and high resolution design shown in FIGS. 15 and 16.

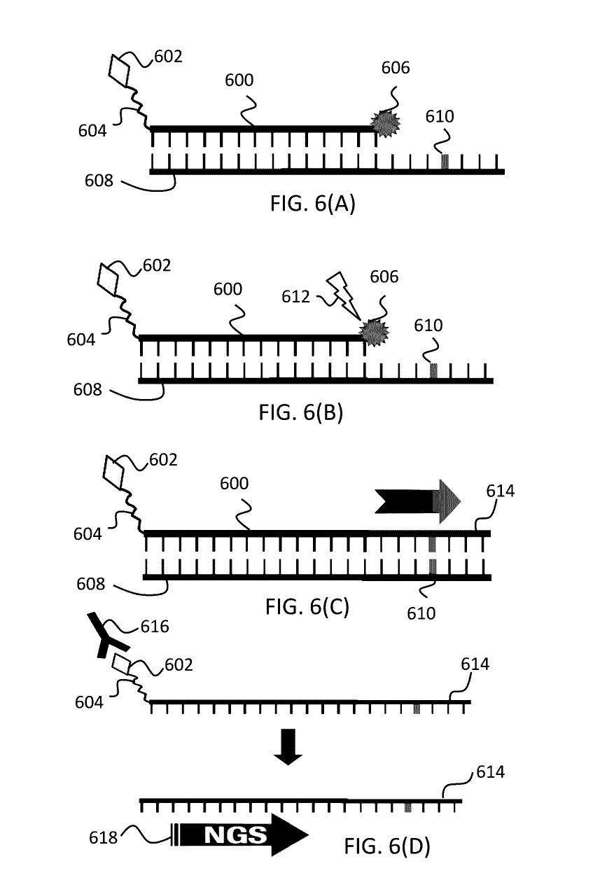

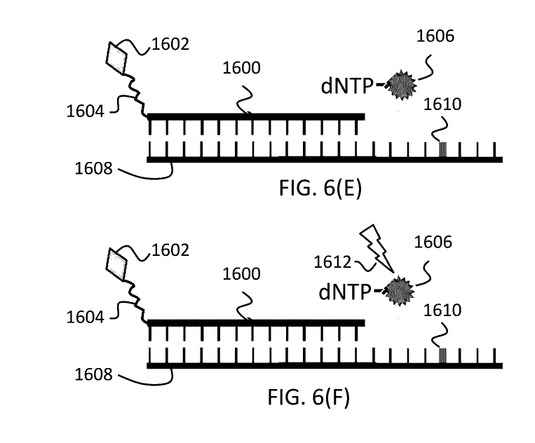

FIG. 6(A)-(D) is a schematic depicting an approach to using photochemistry to facilitate an in situ polymerase extension reaction wherein polymerase enzymes may be added to tissue for in situ polymerase-mediated primer extension. Referring now to FIG. 6(A), oligonucleotide probe 600 is added to a sample having a target sequence 608. Probe 600 includes a photo-reactive moiety (PRM) 606 and optionally a linker 604 and label 602. FIG. 6(B) shows light 612 irradiating PRM 606 so as to enable extension of probe using a polymerase enzyme. In some embodiments, target 608 includes a mutation 610, wherein extension captures mutation information in probe extension 614. In some embodiments, an antibody 616 can be used to purify probe extension. Antibody 616 could be bound to a bead, plate, in gel, etc. and could be selected to bind label 602. FIG. 6(D) shows an arrow 618 representing that probe extension 614 may be analyzed by an appropriate technology, such as next generation sequencing. The analyzed sequence may contain mutation information, a tag sequence (not shown), and the probe sequence.

FIG. 6(E)-(F) is analogous to FIG. 6(A)-(B) and can be used replaceably in the workflow of FIG. 6(A)-(D). According to this embodiment, a probe 1600 and a separate photo-activatable moiety 1606 are used for the photo-activated selection. According to this embodiment, photo-activatable moiety 1606 includes 1 or more of the 4-deoxynucleoside triphosphates (i.e. dNTP), rather than an oligonucleotide probe as shown in FIG. 6(A). Oligonucleotide probe 1600 is added to a sample having a target sequence 1608. Probe 1600 optionally includes a linker 1604 and label 1602. FIG. 6(F) shows light 1612 irradiating photo-activatable moiety 1606 so as to enable extension of probe using a polymerase enzyme. In some embodiments, target 1608 includes a mutation 1610, wherein extension captures mutation information in the probe extension (as shown in FIG. 6(C)). FIG. 6(D) shows an arrow 618 representing that any probe extension may be analyzed by an appropriate technology, such as next generation sequencing. The analyzed sequence may contain mutation information, a tag sequence (not shown), and the probe sequence.

The extent of polymerase chain extension that can be obtained using embodiments of the disclosed method ranges from hundreds of extended nucleotides to thousands of extended nucleotides. In some embodiments, the number of nucleotides added to the initial probe can range from about 10 nucleotides to about 2000 nucleotides (such as about 50 nucleotides to about 1500 nucleotides, or about 100 nucleotides to about 1000 nucleotides, or about 200 nucleotides to about 400 nucleotides). The extent of the chain extension can be determined using one or more reverse primers that have some degree of complementarity (e.g., about 70% to about 99% complementarity) to certain regions of the extended probe. Polymerase-mediated chain extension may be carried out using a range of probe concentrations (e.g., 100 ng/mL to about 100 μg/mL, such as about 10 ng/mL to about 10 μg/mL), and can be carried out for any suitable amount of time, such as from about 20 minutes to about 90 minutes.

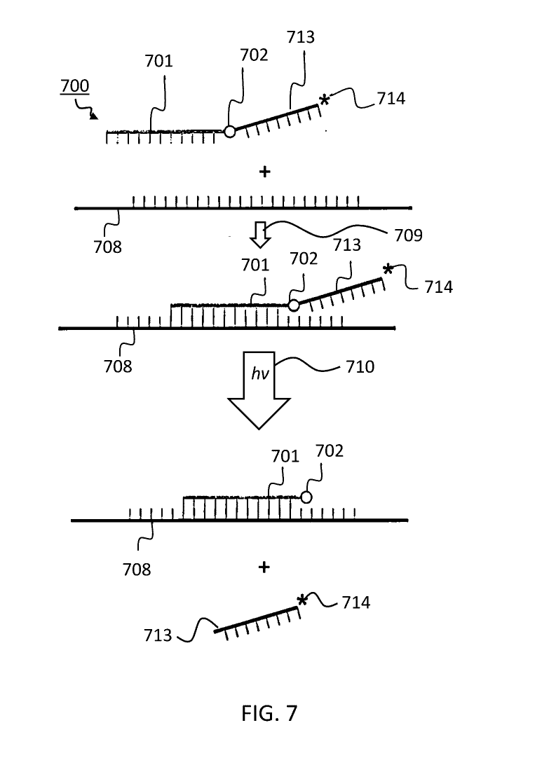

FIG. 7 similarly shows a detection motif within the scope of the present application. In particular, a probe 700 can be brought into contact with a sample having a target 708. Probe 700 comprises specific binding oligonucleotide 701, a photo-cleavable moiety 702, and a detection tag 713. Probe 700 may optionally include a label 714, for example to aid in purification. Contacting probe 700 and sample, under the appropriate conditions results in hybridization of probe 700 to target 708 as indicated by an arrow 709. Subsequent to removal of unbound probe, irradiating a selected portion of the sample with light, as indicated by arrow 710, cleaves photo-cleavable moiety 702, freeing detection tag 713. Detection tag 713 can then be removed from the sample (e.g., through pipetting, contact transfer, absorption, rinsing, microfluidics, rinsing, or other appropriate method), optionally purified (e.g., through use of optional label 714), and analyzed by an appropriate technology (e.g., an array, NGS, PCR, etc.).

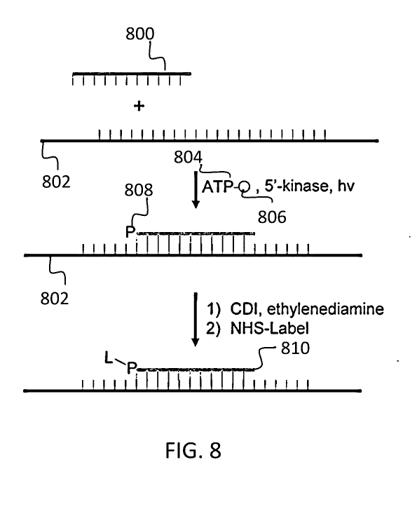

Referring now to FIG. 8, shown is a schematic of a particular embodiment. Oligo/polynucleotide 800 is combined with sample 802. Probe 800 associates with a complimentary sequence sample 802. A composition comprising caged ATP 804 caged with photo-cleavable moiety 806 is added to the sample 802. Irradiation with light liberates caged ATP 804. Exposure to a suitable kinase then phosphorylates probe 800 at the 5′ end to provide phosphorylated probe 808. Subsequent modification of probe 808 may be carried out in order to provide, for example, labeled probe 810.

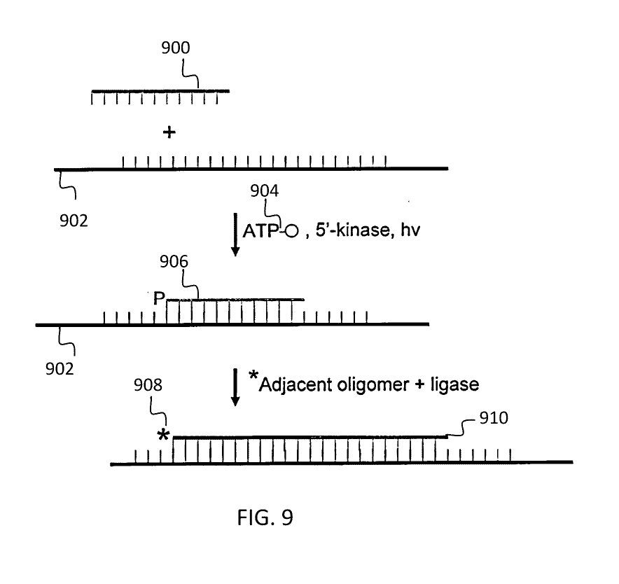

In yet other embodiments, adjacent probes may be linked via a ligase and then a particular tag sequence or unique sequence identifier of one probe may become detectable through ligation to a probe comprising a detectable label. The ligated probes may be amplified using PCR (or ligase chain reaction), if greater sensitivity is desired. A particular embodiment is exemplified schematically by FIG. 9. With reference to FIG. 9, oligo/polynucleotide probe 900 is added to sample 902. Probe 900 associates with a complimentary sequence of sample 902. Sample 902 is then exposed to caged-ATP 904. Activation with light releases the caged ATP. Adding a suitable kinase phosphorylates oligo/polynucleotide probe 900 to provide phosphorylated probe 906. A ligase then ligates oligo/polynucleotide probe 906 to an adjacent oligomer deposited on sample 902 comprising a detectable label 908, thereby forming ligated probe 910. Only those oligomer sequences that are adjacent to the phosphorylated end of oligo/polynucleotide probe 906 will be ligated to the probe. By forming ligated probe 910, a target sequence may be identified once the ligated probe is detected (through detectable label 908) and subsequently quantified using array hybridization or sequencing. In some embodiments, ligase chain reaction or PCR can be used to amplify ligated probe 910 if greater sensitivity is needed.

### 1. Selecting and Preparing the Sample

Samples used in various embodiments of the method, such as a tissue, a cell, or other biological sample (e.g., serum, plasma, and/or urine), can be obtained from a subject for routine screening or from a subject that is suspected of having a disorder, such as a genetic abnormality, infection, or a neoplasia. Samples may be fresh, frozen, formalin-fixed, paraffin-embedded, or combinations thereof. In some embodiments, the method also can be applied to samples that do not have genetic abnormalities, diseases, disorders, etc., referred to as “normal” samples. Such normal samples are useful, among other things, as controls for comparison to other samples. The samples can be analyzed for many different purposes. For example, the samples can be used in a scientific study or for the diagnosis of a suspected malady, or as prognostic indicators for treatment success, survival, etc. Samples can include a single target, or can include multiple targets that can be specifically bound by one or more probe embodiments disclosed herein. While the methods may be used on a variety of samples, it is particularly advantageous for tissue samples which have inherent contextual information that is not present in solution based samples. In particular, the relationship between cells, and indeed the size, shape, and distribution of cells provides an enormous amount of information that is lost if that tissue is lysed, digested, ground. Evidencing the importance of this contextual information is the significance of primary staining (i.e. staining with hematoxylin and eosin, H&E) to the diagnosis of cancer. Being non-specific stains, H&E staining provides only contextual information yet it is the gold-standard in diagnosing cancer.

In particular disclosed embodiments, the sample tissue sample is a formalin-fixed, paraffin-embedded tissue sample. Such tissue samples may be prepared by extracting tissue from a subject, exposing the tissue to a buffered formalin (or paraformaldehyde) solution, and then embedding the tissue with paraffin. The tissue sample(s) may then be placed on a substrate, such as a microscope slide or a microarray slide.

Other embodiments concern cell samples, or soluble protein samples. In some embodiments, the sample may be a smear sample, a cytology sample, a chromosomal smear, a biopsy sample, a fine needle aspirate sample, or a circulating tumor cell sample.

In some embodiments, the sample may be provided on a slide having a registration element suitable for identifying the sample and/or the location/position of the slide comprising the sample. For example, the registration element may be a physical registration element, such as a barcode, alignment holes, alignment protrusions, alignment keys, and the like. The registration elements can facilitate transfer of the slide throughout the system embodiments disclosed herein. For example, the registration elements of the slide can facilitate transfer of a slide comprising a sample to the imaging portion of the system whereby certain areas of the sample are selected and marked for further analysis. The slide can then be processed in other parts of the system, or it can be removed from the imaging component and saved for analysis at a later time. In these embodiments, the registration elements of the slide can be used to reset the slide in substantially the same position as it was taken from the imaging portion of the system, thereby bypassing the need for an additional sample selection step. The slide can be added to the system and undergo subsequent analysis steps without being reimaged and marked.

In some embodiments, the registration elements can be used to translate image based actions (e.g., selection and marking) into actions within the system, such as an irradiation event. The registration element can indicate to the system that the slide is correctly aligned for irradiating the sample in the selected (or non-selected) areas as disclosed herein.

Each slide can be labeled with a barcode number specific to that particular slide and sample. This barcode number can be used to correlate a slide specimen with particularly selected regions of the sample that can be stored electronically. This electronically stored information can be used for subsequent analysis of the particular sample. Accordingly, a number of slide specimens can undergo one or more initial selection steps and then be stored for later analysis.

### 2. Contacting the Sample with the Probe

After a sample is prepared, the biological sample can then be exposed to one or more probes. For example, a sample may be exposed to a probe by contacting the sample with a solution comprising the probe, or a mixture of probes. The solution containing the probe can be made with any biologically-acceptable composition, such as a buffer composition. In some embodiments, the buffer comprises 50 mM Tris/HCl, 10 mM KCl, 5 mM (NH₄)₂SO₄, 2 mM MgCl₂. In other embodiments, the buffer may be selected from those typically used in the art, such as TES, HEPES, Tris, and the like.

In particular disclosed embodiments, the probe is deposited onto a sample region and may be hybridized to, or chemically coupled to (e.g., electrostatically coupled, covalently coupled, or ionically coupled), a particular target sequence or moiety present in the target, or that has also been associated with the target, such as a primary antibody. After the probe is associated with the target, the sample may be washed and any unassociated probe components can be washed away.

In some embodiments, the deposited probe may be detected and particular portions of the sample comprising the deposited probe may be marked for analysis. In some embodiments, visual detection of a signal produced by a detectable label (e.g., a chromophore, a fluorophore, a hapten, etc.) during a primary and/or secondary recognition event can be used to detect the particular portion of the sample to be marked. In some embodiments, one or more portions of the sample may be selected for further analysis by using a stain (e.g., a primary stain, counterstain, or combination thereof). In other embodiments, fluorescence of a fluorophore deposited on one or more portions of the sample may be used to indicate where the sample should be marked for further analysis. In yet other embodiments, morphological characteristics may be used to distinguish portions of a sample for analysis, particularly in cell or tissue samples. For example, fluorescence microscopy and/or phase contrast microscopy techniques known in the art can be used to visualize the shape and/or arrangement of cell components thereby facilitating selection of these particular regions for analysis. Digital pathology techniques also can be used to image the sample and facilitate sample selection.

Once the portion of the sample that will be analyzed has been identified, it may be marked. For example, the portion of the sample that will be analyzed may be manually marked, such as marking a sample portion on a slide to which the sample is attached, or it may be marked on a separate digital viewing screen with the selected locations being stored electronically. In some embodiments, a marking/selection step may involve a substantially instantaneous irradiation event. In other embodiments, a marking/selection step may occur first, followed by a subsequent irradiation step. The act of marking/selecting a portion of the sample may be configured to initiate an irradiation event.

### 3. Exposing the Sample to Light

In illustrative embodiments, a system for performing contextual diagnostics using a photo-reactive probe further comprises a registration system configured to determine the position of the sample, thereby enabling automatic relation of the location inputs to the location on the sample upon which the light source is configured to irradiate. In one embodiment, the system includes a sample platform that is configured to receive the sample adhered to a substrate and the registration system is configured to relate the position of the sample by determining the position of the substrate in relation to the light source. In another embodiment, the substrate is a glass microscope slide inscribed with a registration mark. In another embodiment, the registration system is configured to detect a registration mark. In yet another embodiment, the registration system is configured to detect two or more registration marks, the two or more registration marks inscribed on the substrate. In yet another embodiment, the registration system is configured to identify a location of the registration mark to within about 500 μm, to within about 300 μm, to within about 100 μm, to within about 50 μm, or to within about 10 μm. In yet other illustrative embodiments, the system uses the tissue as a registration mark.

In illustrative embodiments, the registration system is configured so that it enables the computer, which is configured to receive a data file associated with an image of the sample, to relate the image data to the position of the sample in relation to the light source. This in turn, enables user inputs that relate to the image data file to be actionable with respect to the sample. In one embodiment, the registration system is configured to detect a registration mark and the computer is configured to associate the registration mark with an imaged registration mark within the image of the sample. In another embodiment, the registration system is configured to detect two or more registration marks and the computer is configured to associate the two or more registration marks with two or more imaged registration marks within the image of the sample such that the computer is configured to relate the image of the sample with a position of the sample within an irradiation chamber. In yet another embodiment, the computer is configured to accept user inputs, the user inputs include selecting portions of the sample, e.g., based on the image of the sample, for instructing the laser to ablate the sample. In another embodiment, the computer is configured to accept artificial intelligence inputs, the artificial intelligence inputs include data from automated pattern recognition of the image of the sample. In another embodiment, the user is a pathologist and the user inputs are selections of regions of the sample selected by the pathologist from the image of the sample. In another embodiment, the computer is configured to accept a data file associated with an image of the sample and the location inputs are associated with user-selectable regions on the sample for irradiating.

When the sample is exposed to light, the entire sample may be irradiated, or a portion (or portions) of the sample may be selectively irradiated. In some embodiments, the sample is selectively irradiated to facilitate isolation of only certain portions of the probe deposited on the sample (e.g., the unique sequence identifier or tag sequence of a probe) in a targeted region of the sample. In some embodiments, the sample may be selectively irradiated to facilitate isolation of probes deposited on the sample in non-selected regions. That is, selected portions of the sample identified for analysis may be marked and then unmarked portions of the sample to which one or more non-selected probes are bound may then be irradiated, thereby cleaving the non-selected probes. The selected probes may then be isolated using a second irradiation step and/or secondary cleavage technique as disclosed herein and a suitable elution step. In other embodiments, the sample may be selectively irradiated to facilitate isolation of probes deposited on the sample in the selected regions. Selective irradiation can be performed iteratively to provide multiple different irradiation events. In such embodiments, targets and/or detection tags optionally can be isolated between each iterative irradiation step to provide a quantitative, multiplexed map of the sample and the various different targets therein.

Selective irradiation can be accomplished using a photomask, a focused beam of light from a light source, or by iteratively tuning (for a particular photo-cleavable moiety) light of selected wavelengths. For example, selective irradiation can be used to activate a photo-cleavable moiety that is present in the probe and/or other component to which the sample is exposed. In some embodiments, activating the photo-cleavable moiety facilitates cleaving portions of the probe. For example, if the photo-cleavable moiety is used to link two components of a probe, selective irradiation separates the two components.

In some embodiments, irradiation may be used to generate a reactive species that is capable of reacting with other moieties present, such as by cleaving one or more linkers. Suitable reactive species do not interfere with sample analysis or destroy the sample. Suitable reactive species include, but are not limited to, photoinitiators such as azobisisobutyronitrile (AIBN), benzoyl peroxide (BPO). Embodiments of the reactive species can cleave linkers selected from, for example, photo-cleavable moieties used as linkers, aliphatic linkers, alkylene oxide linkers, disulfide linkers, commercially available linkers, polymeric linkers, and the like.

In some embodiments, the photo-cleavable moiety functions as a blocking agent. In these embodiments, selective irradiation cleaves the photo-cleavable moiety from the particular probe (e.g., a primer) and frees at least one end of the probe for further manipulation. An exemplary embodiment is illustrated below in Scheme 5. Conjugate 500 includes a nitrophenyl moiety that readily absorbs light having wavelengths of about 300 nm to about 400 nm, such as about 325 nm to about 375 nm, or about 345 nm to about 370 nm. The nitrophenyl moiety is attached to at least one nucleotide of an oligo/polynucleotide via an ester group. UV irradiation cleaves the benzyl carbon-oxygen bond, followed by release of CO₂ to form conjugate 502, which has a free hydroxyl group suitable for chain extension.

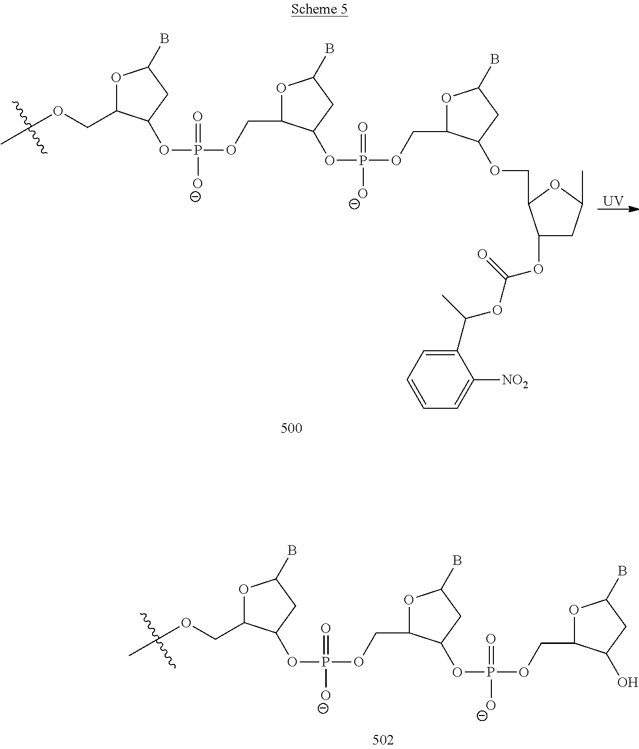

Selective irradiation also can be used to cleave photo-cleavable moieties from a photo-activatable primer extension reagent, such as caged dATP molecules, which are disclosed herein. Other photo-activatable primer extension reagents can include activatable degenerate primers for whole genome amplification or specific genetic lesions, dGTP, dCTP, dTTP, and the like.

Disclosed herein are various different embodiments for irradiating the sample to provide selective target irradiation. During the irradiation step, the sample is exposed to light capable of providing the energy sufficient to induce a desired result, such as to activate the photo-cleavable moiety. In particular disclosed embodiments, the light is ultraviolet light having a wavelength ranging from about 200 nm to about 400 nm. In some embodiments, light is visible light having wavelengths ranging from about 400 nm to about 790 nm. Suitable light sources are capable of providing low intensity light to preserve sample integrity. In some embodiments, the light source provides from about 1 mW/cm² to about 1 W/cm², such as from about 1 mW/cm² to about 500 mW/cm², or from about 1 mW/cm² to about 200 mW/cm², or from about 5 mW/cm² to about 150 mW/cm², or from about 10 mW/cm² to about 150 mW/cm². Exemplary light sources include, but are not limited to, lasers, LEDs, or lamps (such as incandescent lamps, halogen lamps, mercury lamps, fluorescent lamps, and the like).

In illustrative embodiments, the irradiated tissue area would be selected dependent on the surface area of diseased tissue, more specifically the number of diseased cells. In many cases, such as biopsies, this area would range from a single cell (10-20 micrometers in diameter) to a few thousand cells (1-50 millimeters in diameter). In resected tissues, the diseased area may include hundreds of thousands to millions of cells covering the majority of a glass slide (75×25 mm). In yet other embodiments in which a DMD is used, the irradiated area could be about 10 μm×10 μm or larger regions made up by 10 μm×10 μm regions

Selective irradiation may comprise direct irradiation or indirect irradiation. Direct irradiation typically comprises exposing the sample to light, particularly a light source capable of producing light of any suitable wavelength (or wavelengths), without filtering the light produced by the light source. For indirect irradiation, an intermediate filter or masking material may be placed between the light source and the sample, thereby filtering or blocking some of the light produced by the light source from the sample.

Selective irradiation also may comprise exposing the sample to light positioned to selectively expose a particular portion of the sample. For example, an LCD screen may be used to direct light of a selected wavelength (or wavelengths) to a particular location in the sample. A tumor-containing portion of a tissue may be irradiated and a non-tumor-containing portion of the sample may be irradiated (typically sequentially). These particular embodiments provide the ability for collecting portions of a probe (e.g., a tag-template duplex or unique sequence identifier) that are liberated by the selective irradiation (via cleavage of the photo-cleavable moiety) and sequencing a tag-template duplex or unique sequence identifier that correlates to the tumor portion of the sample and a different tag-template duplex or unique sequence identifier that correlates to the normal portion of the sample. In some embodiments, selective irradiation can be used to cleave a pull-down tag in an unselected tissue area (e.g., an area that does not comprise a tumor). For example, a detectable label (e.g., biotin) attached to a sample through one or more probe components can be cleaved from regions of the sample other than the tumor-containing area.

FIG. 5(A)-(C) are schematic drawings showing several approaches to irradiating a first selected region 504 and/or a second selected region 503 of a sample 502 disposed on a slide 501. Referring now to FIG. 5(A), a microscope objective 511 can be used to view the sample so that a region of interest can be selected. The microscope objective can also be used to direct light irradiating the sample. For instance, region of interest 504 may be selected in the view of objective 511. Light from an appropriate source can then be directed through objective 511 to region 504. The effect of this approach is irradiation of the field of view, as such, the field of view for the microscope is the selected region. Referring now to FIG. 5(B), shown is an alternative approach that uses a light source 520 to irradiate a screen 521 having selective optical density. Screen 521 can block incident irradiation to the bulk of sample 502 while allowing irradiation to impact regions 504 and 503 as depicted by beams 522 and 523 passing through the screen to the respective selected regions.

The selective optical density screen may be a liquid crystal display (LCD) screen. The sample regions of interest may be marked by configuring an LCD screen to have the desired area outlined (for example, the positions may be marked manually, and the particular area/pattern transferred to the LCD screen using a projector). The areas that are not to be irradiated are “blacked-out.” Portions of the LCD screen that are not “blacked-out” enable passage of the light produced by the light source to the selected region. Referring now to FIG. 5(C), in another embodiment, a digital micromirror device 531 is used selectively irradiate regions 504 and 503. Beams 532 and 533 are generated by irradiating digital micromirror device 531 with light source 520. In particular disclosed embodiments, the digital micromirror device comprises a 1400×1050 array of micro-mirrors (12.88 μm²).

In yet other embodiments, selective irradiation may be obtained by using a digital micromirror device, which can be used to focus light from the light source onto the sample. The digital micromirror device may comprise various different portions/mirror segments of a particular size (e.g., squares having a size ranging from about 4.5×4.5 μm to about 15×15 μm) that can be adapted to irradiate particular locations and/or features within the sample.

The sample typically is exposed to the light for a time sufficient to cleave the desired photo-cleavable moieties, but not so long as to effect sample degradation. For example, the time period during which the sample is irradiated typically is no longer than about 5 minutes, about 4 minutes, about 3 minutes, or about 2 minutes. In some embodiments, the sample may be irradiated for an effective period of time ranging from about 1 second to about 60 seconds, typically from about 10 seconds to about 60 seconds, more typically from about 20 seconds to about 60 seconds, with certain working embodiments irradiating the sample for about 20 seconds or less.

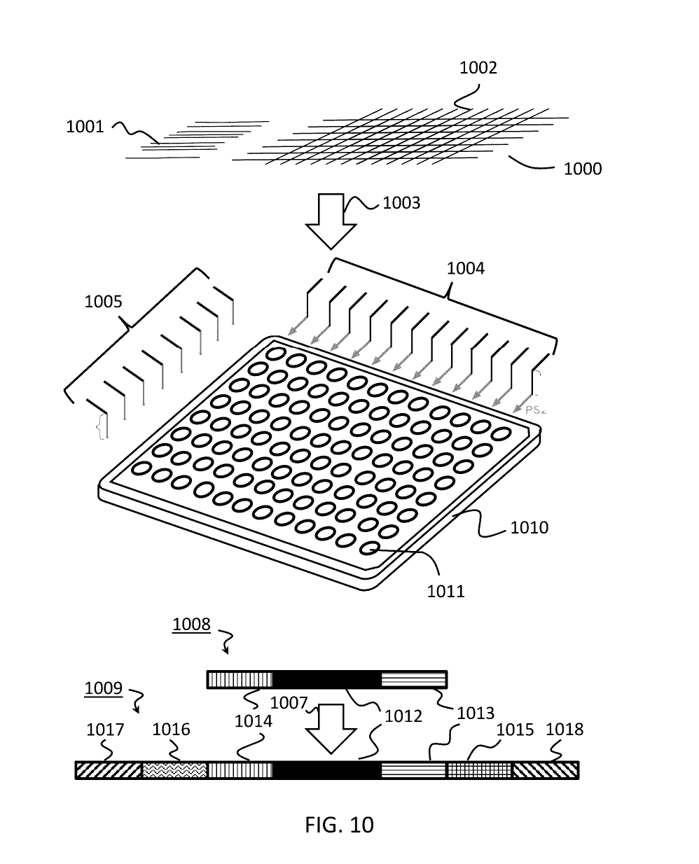

Referring now to FIG. 10, shown is a schematic that illustrates an embodiment of the present disclosure wherein a multiplexed image of the sample is constructed using the approaches described herein. Across a substrate 1000, for example a microscope slide labeled with a barcode 1001, a grid 1002 or other appropriate sectioning strategy is created to overlap the sample (not shown, but understood to be within the grid). Grid 1002 is arranged to correspond to the resolution of the chemical image that is sought. Illustrative of the method, the grid may be a series of raster locations on which the sample is iteratively irradiated. The arrangement and structure of the grid is not material, rather it can be variably designated according to the intended need. In some samples, specific cells are irradiated and the “grid” does not follow a geometric pattern, but instead matches a biologic pattern associated with the sample. Referring back to FIG. 10, arrow 1003 represents removal of detection tags from substrate 1000 to a multi-well plate 1010. Multi-well plates are well known in the art and come in various configurations having various numbers of wells from 2 to hundreds. While shown as a typical macroscopic (e.g., 64-well plate), microscopic vessels are also within the scope of the present disclosure. For example, multi-well plate may be a microfluidic device having small volume chambers. Multi-well plate 1010 is shown to include a plurality of reaction wells 1011.

According to illustrative embodiments, a light is used to irradiate a selected region (e.g., a single grid location), thereby releasing detection tags from the selected region. The detection tags are removed from substrate 1003, as represented by arrow 1003, and deposited into one of the plurality of reaction wells 1011. The process is repeated iteratively so that detection tags from each selected region are deposited into a different reaction well. While the detection tags present in each cell could be independently analyzed, a more elegant solution is shown in FIG. 10. According to this solution, different forward primers (represented as arrows 1005) can be placed in each row of multi-well plate 1010. Similarly, different reverse primers (represented as arrows 1004) can be placed in each column of multi-well plate 1010. Within each well, detection tag 1008 (represented in a block diagram), includes a portion 1014 corresponding to a portion of forward primer 1005 and a portion 1013 corresponding to a portion of reverse primer 1004. Arrow 1007 represents a PCR step which amplifies the detection tags in each well and incorporates forward and reverse primers to create a PCR product 1009. PCR product 1009 may include a first adapter sequence 1017 (e.g., for NGS), a unique row identifier 1016, the portion 1014 corresponding to the portion of the forward primer 1005, a unique identification tag sequence (e.g., which would include target or reagent information), the portion 1013 corresponding to the portion of the reverse primer 1004, a unique column identifier 1018, and a second adapter sequence 1018 (e.g., also for NGS). The elegance of this approach is that all the wells could then be pooled and analyzed concurrently. The location from where the detection tag came on the sample is encoded in the sequence. As such, all detection tags from each location on the sample could be binned together to read out a highly multi-plexed chemical image of each selected region of the sample.

### 4. Detecting the Tag and/or the Target

The target may be detected using various detection schemes now known or hereafter developed. In particular disclosed embodiments, detecting may first comprise obtaining an isolated portion of the probe and analyzing/sequencing the isolated portion of the probe. For example, after the photo-cleavable moiety is cleaved, a portion of the probe may be released and the released portion isolated from the sample, such as by washing the sample with an appropriate buffer solution. If the isolated portion includes information encoded by the nucleotide sequence, the isolated portion is then sequenced, such as by using next generation sequencing. If necessary, or desirable, an isolated portion of the probe may be amplified, such as by performing a polymerase chain reaction. In particular disclosed embodiments, amplifying and sequencing steps may be combined. In some embodiments, portions of the sample that are irradiated may undergo amplification while portions of the sample that are not irradiated are not amplified.

Next generation sequencing provides the ability to sequence a large number of samples, wherein each sample may comprise multiple targets and/or specific binding moieties bound to unique sequence identifiers. As each unique sequence identifier is different, each of the multiple different targets and/or specific binding moieties can be bound to its own unique sequence identifier. Once isolated, unique sequence identifiers can be differentiated during sequencing and the particular target and/or specific binding moiety identified based on its correlated unique sequence identifier. Thus, the identity of the target and/or specific binding moiety may be detected with increased accuracy as compared to typical tissue analytical techniques. Additionally, the quantity of specific binding moieties bound in a particular portion of a sample may be determined based on the isolated unique sequence identifiers.

In some embodiments, the liberated unique sequence identifier may be collected out of a suitable microfluidic platform after sample irradiation. The microfluidic platform may be a microarray platform, such as a microarray slide. In some embodiments, a microarray slide can be used comprising a surface designed to spatially arrange particular targets in locations that facilitate probe addition and selective retrieval of moieties that are cleaved from the probe (e.g., unique sequence identifiers, tag sequences, primers, and the like). The microarray slide can comprise multiple, spatially arranged locations that can comprise one particular target, multiple targets, or subsets of targets. Due to the spatial arrangement on the microarray slide, a selected region containing a portion of the moieties that are cleaved from the probe after selective irradiation can be eluted without eluting substantial amounts of non-selected regions. The selected region may be exposed to an elution buffer and/or a denaturing buffer. These buffers facilitate removal of the desired moieties within the selected region (e.g., unique sequence identifier, tag sequence, primer, etc.). The microarray slide typically may be used in combination with a microarray scanner that is configured to scan the microarray slide's surface and identify the region of interest. A dispensing instrument also may be used, which can be configured to receive input from the microarray scanner and dispense the desired elution and/or denaturing buffers to a particular region of the slide. An exemplary microarray system comprising these components (and other suitable components) is disclosed in U.S. Patent Publication No. 2010/0076185, which is incorporated herein by reference in its entirety.

### B. Photochemical Immunohistochemical Analysis

In particular disclosed embodiments, the method may comprise a histochemical assay suitable for detecting one or more targets in a sample. The probes used in these embodiments typically comprise an antibody as the specific binding moiety, a photo-cleavable moiety, and a tag sequence or a unique sequence identifier. Particular embodiments of the method concern exposing a biological sample, such as a tissue sample (e.g., an FFPE tissue sample), to one or more probes comprising an antibody that can either bind directly to a target in the sample, or indirectly to a target. For example, particular embodiments concern first contacting the sample with a primary antibody, followed by contacting the sample with a probe comprising a secondary antibody, a photo-cleavable moiety and a unique sequence identifier (or tag sequence), where the secondary antibody specifically recognizes the primary antibody. If a unique sequence identifier is used, then it can be released via selective irradiation and the sequence determined to identify the target detected. This embodiment also can be used to quantify the number of bound antibodies in the tissue as the number of sequencing reads can equate directly to the number of bound antibody-unique sequence identifier conjugates.

### C. Photochemical In Situ Hybridization

Also disclosed herein are embodiments of a method wherein one or more targets are detected using a photochemical-based in situ hybridization assay. In particular disclosed embodiments, the method concerns exposing a sample to a probe comprising an oligo/polynucleotide, a photo-cleavable moiety, and a tag sequence or unique sequence identifier. The probe is allowed to hybridize to the tissue sample and then portions of the sample are irradiated using a light source.

In other disclosed embodiments, the method may concern exposing a sample to a probe comprising an oligo/polynucleotide, a photo-cleavable moiety, and a first member of a binding pair, typically selected from biotin, streptavidin, or a hapten. After the probe is hybridized to the particular target of interest, the sample is washed and then certain portions are irradiated with light to dissociate a first member of the specific binding pair from any hybridized probes within the particular zone of irradiation. After performing a dehybridization step on the sample, the de-hybridized probes are passed through a column comprising an immobilized second member of a specific binding pair. Probes that were irradiated elute through the column, as specific binding cannot occur between the probe and the second member of the specific binding pair present on the column. Those probes that were not selectively irradiated will not elute through the column, as the still-attached first member of the specific binding pair binds with the second member and is retained on the column. In some embodiments, the method facilitates isolation of the probes located in the particular regions of interest.

### D. In Situ Polymerase Extension

In particular disclosed embodiments, photochemistry may be used to facilitate an in situ polymerase extension reaction wherein polymerase enzymes may be added to tissue for in situ polymerase-mediated extension, in situ PCR, and/or in situ nick-translation. For example, in particular embodiments, a nucleic acid target with a point mutation may be analyzed by exposing the nucleic acid target to a probe comprising an oligo/polynucleotide and a photo-cleavable moiety that blocks one end, typically the 3′ end, of the oligo/polynucleotide. The oligo/polynucleotide may further comprise a detectable label.

In another embodiment one or more of the deoxynucleoside triphposphates are blocked with a photo-cleavable moiety and the oligo/polynucleotide may or may not be blocked with a photo-cleavable moiety.

The extent of polymerase chain extension that can be obtained using embodiments of the disclosed method ranges from hundreds of extended nucleotides to thousands of extended nucleotides. In some embodiments, the number of nucleotides added to the initial probe can range from about 10 nucleotides to about 2000 nucleotides (such as about 50 nucleotides to about 1500 nucleotides, or about 100 nucleotides to about 1000 nucleotides, or about 200 nucleotides to about 400 nucleotides). The extent of the chain extension can be determined using one or more reverse primers that have some degree of complementarity (e.g., about 70% to about 99% complementarity) to certain regions of the extended probe. Polymerase-mediated chain extension may be carried out using a range of probe concentrations (e.g., 100 ng/mL to about 100 μg/mL, such as about 10 ng/mL to about 10 μg/mL), and can be carried out for any suitable amount of time, such as from about 20 minutes to about 90 minutes.

### E. Multiplexing

The components, methods, kits, and systems described herein primarily provide description for one or two probes used on a particular sample. While useful in this context, one significant advantage of the present approach is that the detection modality is based on oligonucleotides. As such, the detection and quantification of binding events is performed using robust and well-known oligonucleotide approaches which are capable (and routinely applied to) high levels of multiplexing. In illustrative embodiments, between about 2 and about 100,000 targets are detected. In other embodiments, between about 10 and about 100,000 targets are detected. In another embodiment, between about 10 and about 1000 targets are detected. In yet another embodiment, greater than about 5 targets are detected. In another embodiment, greater than about 10 targets are detected.

### VI. System Embodiments

Also disclosed is a system for implementing any of the method embodiments disclosed herein. In some embodiments, the system comprises a slide tray configured to accept a slide comprising a sample, a dispenser configured to dispense disclosed probe embodiments, such as a probe comprising a specific binding moiety, a photo-cleavable moiety, and a detection tag. The system also includes a light source configured to selectively irradiate the sample, thereby cleaving the photo-cleavable moiety and releasing the detection tag. The slide used in the disclosed system may be a single specimen slide, an array slide, or a microarray slide. The light source may be selected from any of the light sources disclosed herein, such as a laser, an LED, or a lamp.

In some embodiments, the disclosed system further comprises an imaging device configured to capture a modifiable sample image, a digital screen configured to accept the modifiable sample image from the imaging device, and a selection device configured to mark a selected portion of the modifiable sample image for analysis. The imaging device may be a digital camera or a video camera.

The disclosed system also may further comprise a dispensing instrument configured to dispense an elution buffer onto the slide to elute the detection tag, and a recovery instrument configured to recover the detection tag. The recovery instrument may be configured to deliver the detection tag directly to an additional portion of the system. For example, the system may further comprise a PCR instrument configured to accept and amplify the detection tag. Other embodiments further comprise a sequencing instrument configured to accept and sequence the detection tag, and a computer configured to translate information generated from the sequencing instrument to the digital screen, thereby correlating the detection tag to the selected region from which it was cleaved. Yet other embodiments further comprise an array platform comprising a modified surface configured to accept and bind the detection tag.

Exemplary automated systems available through Ventana Medical Systems, Inc., Tucson, Ariz. include SYMPHONY® Staining System, catalog #: 900-SYM3, VENTANA® BenchMark Automated Slide Preparation Systems, catalog #s: N750-BMKXT-FS, N750-BMKU-FS, VENTANA, and VENTANA® BenchMark Special Stains automated slide stainer. These systems employ a microprocessor controlled system including a revolving carousel supporting radially positioned slides. A stepper motor rotates the carousel placing each slide under one of a series of reagent dispensers positioned above the slides. Bar codes on the slides and reagent dispensers permits the computer controlled positioning of the dispensers and slides so that different reagent treatments can be performed for each of the various tissue samples by appropriate programming of the computer.

Illustrative instrumentation systems are designed to sequentially apply reagents to tissue sections mounted on one by three inch glass microscope slides under controlled environmental conditions. The instrument must perform several basic functions such as reagent application, washing (to remove a previously applied reagent), jet draining (a technique to reduce the residual buffer volume on a slide subsequent to washing), application of a light oil used to contain reagents and prevent evaporation, and other instrument functions. Exemplary staining instruments process slides on a rotating carousel. The slides maintain a stationary position and a dispenser carousel rotates the reagents above the fixed slides. The processes described herein can be performed using various physical configurations. The process of clarifying and staining tissue on a slide consists of the sequential repetition of basic instrument functions described above. Essentially a reagent is applied to the tissue then incubated for a specified time at a specific temperature. When the incubation time is completed the reagent is washed off the slide and the next reagent is applied, incubated, and washed off, etc., until all of the reagents have been applied and the staining process is complete.

For related disclosure, reference is made to Richards et al. U.S. Pat. No. 6,296,809, assigned to Ventana Medical Systems, which describes an apparatus and methods for automatically staining or treating multiple tissue samples mounted on microscope slides so that each sample can receive an individualized staining or treatment protocol even when such protocols require different temperature parameters. More specifically, what is described is an apparatus comprising a computer controlled, bar code driven, staining instrument that automatically applies chemical and biological reagents to tissue or cells mounted or affixed to standard glass microscope slides. A plurality of slides are mounted in a circular array on a carousel which rotates, as directed by the computer, to a dispensing location placing each slide under one of a series of reagent dispensers on a second rotating carousel positioned above the slides. Each slide receives the selected reagents (e.g. DNA probe) and is washed, mixed, and/or heated in an optimum sequence and for the required period of time. The instrument would have a micro-fluidic device that covers the slide using a gasket to keep volumes low. It would also contain micro-pipettes to aspirate the liquid off the slide and into a collection vial following UV illumination. VII, Kits

Also disclosed herein are embodiments of a kit for use in disclosed systems. An exemplary kit for performing contextual diagnostics are is disclosed herein. The kit can comprise obvious components used to practice disclosed embodiments, such as a probe reagent solution comprising a specific binding moiety, a photo-cleavable moiety, and a detection tag.

### VIII. Advancements Over the Art

Embodiments of the methods disclosed herein provide advancements over methods currently used in the art for histopathology. In some embodiments, the disclosed method provides quantifiable results because labels and probes used in embodiments of the method are directly detected and do not require further amplification. Embodiments of the disclosed method are suitable for multiplexing assays as the detection tags used in certain embodiments of the method are uniquely distinct with an infinite number of different species available for use in the disclosed probes. Many multiplexing methods known in the art are limited by the number of detectable tags that can be attached to ISH probes and/or the type of detection system used for immunohistochemistry (IHC) analysis (e.g., colorimetric and/or fluorescent detection schemes). While methods exist to address such limitations, these methods typically require disassociating and lysing the tissue sample, thereby destroying all tissue context. The method disclosed herein avoids these undesirable steps and provides a level of maintained tissue contextual information that is important in IHC and ISH analysis. The disclosed method also provides the benefit of preserving tissue samples for repeated analyses, archiving, and/or conformational analysis. The disclosed method not only facilitates multiplexing assays, but certain embodiments further provide the ability to repeat multiple distinct multiplexing assays on different portions of a particular sample. Methods currently known in the art are unable to address tissue heterogeneity in the same way.

Embodiments of the disclosed method also offer levels of specificity and sensitivity that far exceed methods currently known in the art. For example, some embodiments concern using primer extension techniques to produce detectable moieties that can be isolated from a sample for further analysis. Such in situ extension can be conducted solely on the particular gene of interest, whereas all other sequence reads can be ignored.

In addition, embodiments of the disclosed method provide higher spatial resolution than is achieved with tissue microdissection methods used in the art. Methods disclosed herein also facilitate probing cellular structure and rather than solely the cell itself. The methods disclosed herein also can be automated, thereby providing fast, efficient, methods that can be conducted on bench-top automated devices.

### IX. Working Embodiments

### Probe Design

In some embodiments, oligonucleotide DNA probes were synthesized with a 3′ binding sequence to either a chromosome 17 alpha satellite repeat sequence (SEQ ID NO: 4) or an ALU repetitive sequence (SEQ ID NO: 5), and a 5′ non-binding sequence. In some experiments, a nick-translated, DIG labeled clone of the a7t1alpha satellite repeat from the centromeric region of chromosome 7 was used as a non-UV sensitive control probe. Probes used for demonstration of tissue irradiation were designed with a 5′ non-binding portion that contained two DNP (dinitrophenol) haptens (DNP-TATTTT-DNP-TATTTT), whereas probes used to demonstrate PCR detection of a DNA barcode were designed with a non-binding 5′ tail that contained priming sites for M13 primers (Forward: 5′ being SEQ ID NO: 6, Reverse: 5′ being SEQ ID NO: 7) flanking a DNA unique sequence identifier (SEQ ID NO: 1). The two sequence elements were linked via a photo-cleavable moiety (Ambergen).

### In Situ Hybridization:

In Situ Hybridization (ISH) procedures were executed on a BENCHMARK XT automated slide stainer. Probes were hybridized at 42° C. for two hours in a formamide containing buffer. Probes were washed, and slides were removed from the staining instrument and irradiated. Following UV irradiation, the hybridized probes were detected using Ventana SISH detection on a Benchmark XT instrument.

### UV Irradiation:

An LED light source emitting at 365 nm (XeLED-Nil UV-R3-365, Xenopus Electronix) was used to irradiate the tissue at a distance of 2 cm for 30 seconds. Aluminum foil was placed over the tissue section to block the UV light. Subsequent irradiation methods used UV light from a metal-halide lamp (X-Cite, Lumen Dynamics) passed through a 20× microscope objective. UV energy doses ranged from 50-10 mW for ten seconds to one minute. Intensity of the DMD device UV source was 130 mW/cm2. The slides were irradiated for 20 seconds.

UV irradiation using an LCD screen also was used to shine a pattern on the tissue. The field diaphragm of a microscope using Kohler irradiation was replaced with a liquid crystal panel to turn the microscope into a projector. This allowed controlling which regions of the slide to irradiate by masking specific regions of this tissue from irradiation. For tissue irradiation, the LCD panel was transitioned into a checkerboard pattern. Slides were irradiated using 100 mW for 30 to 60 seconds.

### Example 1

To determine whether UV-cleavable DNA oligonucleotides are suitable DNA probes, a CHR-17 oligonucleotide was designed with a photo-cleavable moiety between the target binding region and the detection region, which comprised detectable DNP labels. The probes were hybridized to a tissue sample; the slides were taken off the instrument and then irradiated with UV light. The slides were then put back on the instrument and the presence of the UV-cleavable DNA tag was determined using a silver ISH [SISH] detection kit. A control also was used with a nick-translate DIG CHR-7 probe.

The results indicated the ability of a probe containing a photo-cleavable moiety to be selectively irradiated and therefore useful in detecting a DNA target in a tissue sample. No DNP tags were detected in the control sample as the portion of the sample was exposed to light and all DNP tags were cleaved with the photo-cleavable moiety. In comparison, the masked areas non-irradiated portions) of the sample did contain DNP-labeled probes as the photo-cleavable moiety in these probes were not cleaved. The DNP labels attached to the probe through the photo-cleavable moiety were therefore detectable using SISH detection.

### Example 2

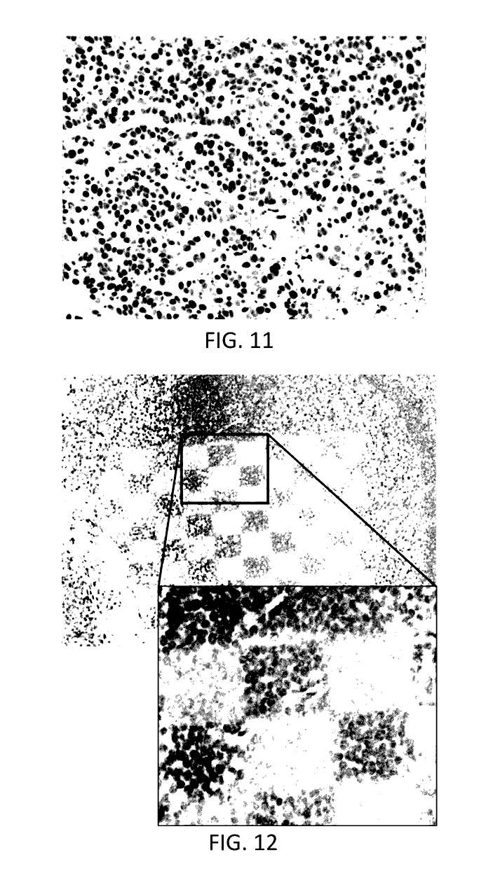

In this embodiment, an ALU DNA ISH probe (comprising an oligonucleotide, photo-cleavable moiety, and tag sequence, labeled with DNP label) was designed to deposit a significant amount of SISH signal off of a DNA hapten attached to a DNA detection region of a DNA probe. The probe was hybridized to various spots on the tissue sample, excess probe was washed, and the slides comprising the tissue samples were removed from the automated stainer and irradiated using one of three methods. In one embodiment, a sample was irradiated at 50 mW for approximately 20 seconds using a 20× objective. FIG. 11 provides an image of a control ALU probe that was not irradiated with light.

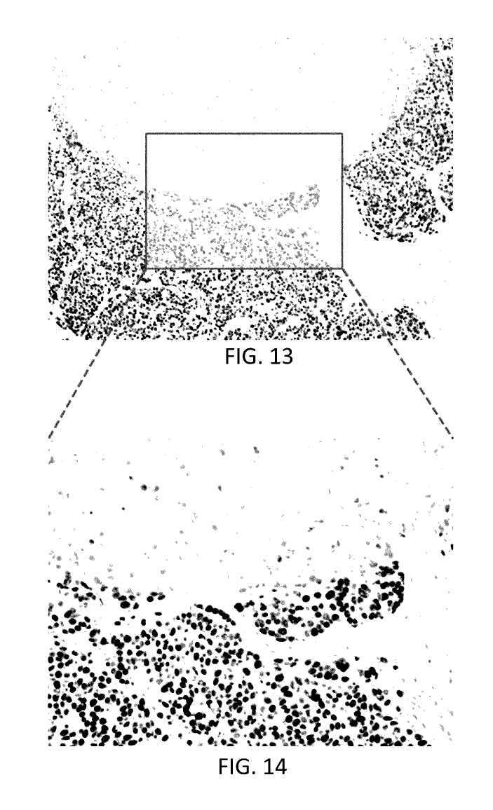

An additional, different sample slide was exposed to a different UV irradiation method. In this embodiment, an LCD screen was placed between a UV lamp and the slide. The LCD display was transitioned to provide a pattern by projecting a patterned slide and thereby making the UV light in the blacked-out portions of the pattern. The slide was irradiated for approximately 60 seconds and provided the patterned sample, to which a photomicrograph is shown in FIG. 12. As shown in FIG. 12, portions of the sample that were exposed to light did not provide a detectable signal, as the DNP tags attached to the probe through a photo-cleavable linker were detached from the sample, whereas those portions that were not irradiated did provide a detectable signal produced by SISH detection of the DNP tags. Additional embodiments are illustrated in FIGS. 13-16, which also show that embodiments of the disclosed method can be used to obtain patterned samples using selective irradiation. In particular, FIGS. 13 and 14 show two levels of magnification for a region that had been directly irradiated using a microscope objective. The spherical aspect of the irradiation is clearly observed in FIG. 13, while the contrast and sharpness of the interface between the irradiated and non-irradiated sections can be seen in FIG. 14.

FIGS. 15 and 16 show the versatility in shape and provide an indication of the resolution which can be accomplished using a light source with a digital micromirror device (DMD). In particular, FIG. 15 shows that an elaborate symbol can be illuminated while FIG. 16 shows the resolution of the approach, where each of the speckles represents quality control patterns associated with the DMD.

### Example 3

In this particular embodiment, a CHR-17 oligonucleotide was used as a primer for DNA polymerase I. The probe was hybridized to tissue, which was then washed. Using nick-translation protocol, DNA polymerase I (e.g., 10, 1, or 0.1 units of DNA polymerase per slide) and other extension reagents were added to the sample to extend the 3′ end of the CHR-17 oligonucleotide, with an extension time of 2 hours. DIG-dUTP was then added to detect primer extension using an anti-DIG, AP Red detection kit. The results for these particular examples indicated that polymerase chain extension had occurred as the AP red labels were detected in the sample.

### Example 4

In this particular embodiment, a DNA probe, comprising a photo-cleavable moiety and a unique sequence identifier (comprising a primer portion, a sequence identifier portion, and a second primer portion) was used. The DNA probe was hybridized to tonsil tissue on a BenchMark XT instrument. The slides were then removed and irradiated with UV light at 50 mW for approximately 20 seconds using a 20× objective. Liquid on the slides containing the liberated unique sequence identifiers was collected and used as a template in subsequent PCR reactions.

### Example 5

In one embodiment, a biotinylated secondary antibody was bound to a biotinylated tag sequence using streptavidin. An antigen retrieved target was first labeled with a primary antibody, followed by binding with the biotinylated anti-species secondary antibody. Streptavidin was used to connect the biotinylated tag with the biotinylated antibody. After a stringent wash, the tag was hybridized to a template oligonucleotide. The slide was then treated with exonuclease. All steps were performed on a VMSI Benchmark XT stainer. After washing and air-drying, the template was transferred into a solution for PCR quantification.

Series dilutions of two particular template oligonucleotides were made and the quantification dynamic range determined by using corresponding primers. A seven orders of magnitude linear range was achieved with the detection of 120 molecules possible (2 μL of 0.1 fM). Run-to-run reproducibility also was demonstrated. There was little discrepancy between the two template oligonucleotides and therefore it is possible to develop multiplexed assays by using different template oligonucleotides with the same probe. The detection and quantification of the template oligonucleotides with sufficient sensitivity and linear dynamic range have been demonstrated. In some embodiments, a specific template was quantified even in the presence of other template oligonucleotides using a corresponding unique primer.

### Example 6

In this particular embodiment, ALU and CHR17 oligo/polynucleotide sequences (SEQ ID NO: 5 and SEQ ID NO: 4, respectively) attached to the three unique sequence identifier sequences (SEQ ID NOS: 21-23) via a photocleavable linker (commercially available from TriLink, structure provided below) were designed. The probes (1 μg/ml final concentration) were hybridized to tonsil tissue and were exposed to UV irradiation using a digital mirror display (DMD) irradiation technique. Irradiation was carried out using the entire DMD array field (19,151×14,363 μm) with a UV intensity of 130 mW/cm². Control slides were left untreated with UV light.

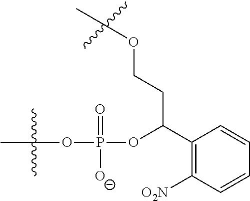 [SMILES: *OCCC(OP(=O)([O-])O*)c1ccccc1[N+](=O)[O-]]

### Photocleavable Linker

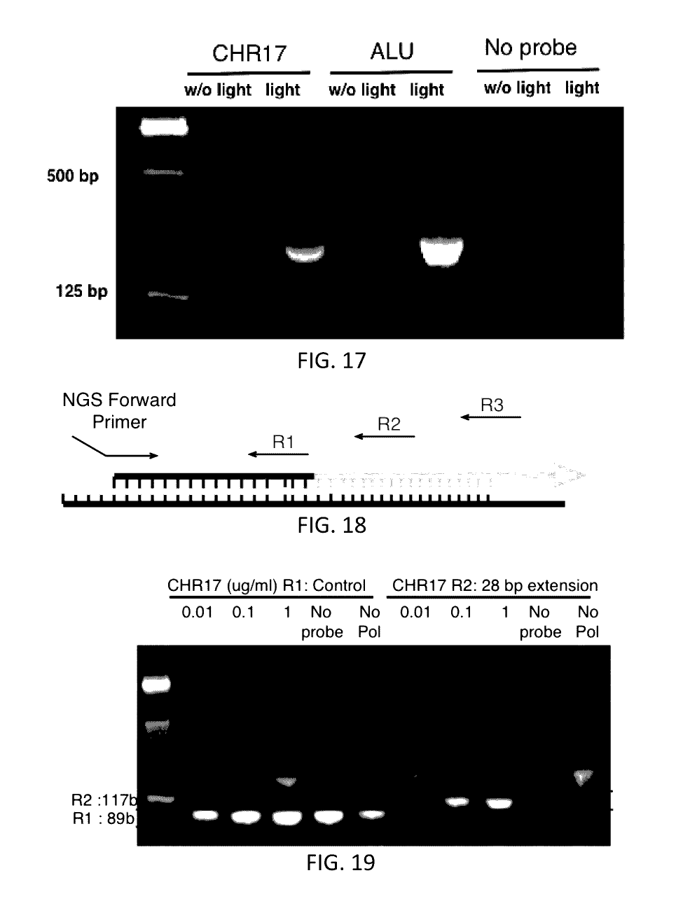

After the irradiation, the liberated unique sequence identifiers were released into 200 μL of TE buffer solution and collected using a microfluidic platform. PCR amplification was performed in a 50 μL of total reaction using 20 μL of the solution comprising the unique sequence identifiers as a template and NGS specific (Illumina) adaptor sequences (SEQ ID NOS: 26 and 27) as primers. Amplification was performed using the following thermal cycling conditions: 98° C. for 10 seconds, 60° C. for 10 s, 72° C. for 1 min (15-20 cycles). The amplified unique sequence identifiers with Illumina adaptors were analyzed using gel electrophoresis (Invitrogen 4% EZ Gel). The results are shown in FIG. 17. As shown in FIG. 17, no signal was obtained for control samples that were not irradiated (and therefore did not provide unique sequence identifiers capable of being isolated) and for control samples that were not exposed to the probe comprising the unique sequence identifiers. Bands were obtained, however, for the unique sequence identifiers (attached to the NGS specific adaptor sequences) that were exposed to light.

Furthermore, we demonstrated the detection of single and multiplexed DNA barcodes corresponding to diagnostic biomarkers via Illumina Sequencing. In particular, ALU, CHR7 and CHR17 probes attached to the three unique barcode sequences were hybridized to the tonsil tissue separately and/or in multiplex. After washing, the barcodes were then released into the solution by UV illumination. The Illumina adaptors were attached to the barcodes in a PCR reaction using the solution containing the barcodes as the template (as described previously). Each PCR sample was purified, and the gel band of the target size (211 bp) was selected on 2% E-gel Size Select agarose gel (Invitrogen) resulting in sequencing-ready library for Illumina paired-end sequencing. The library was then quantified using the PicoGreen assay (Invitrogen Molecular Probes) and multiplexed with other libraries and ran on a Illumina MiSeq instrument (150×150 paired end).

The counts from the sequencing run were mapped using BOWTIE2 with a 68.79% overall alignment rate (Table 1). In this particular experiment, barcode counts corresponding to CHR7 and CHR17 are in multiplex, i.e., the probe cocktail containing CHR7 and CHR17 probes were hybridized simultaneously on the same tissue slide. The barcode counts corresponding to ALU is from a separate single ISH experiment. The libraries were multiplexed and analyzed on a single MiSeq run.

TABLE 1

| | Centromeric | MiSeq | Binding |
|---|---|---|---|
| Barcode | Probe | Counts | events/cell |
| BC1 | CHR7 | 37602 | ~200-1000 |
| BC2 | CHR17 | 118013 | ~3000 |
| BC3 | ALU | 1398884 | ~1,000000 |

The barcode counts corresponding to CHR7 and CHR17 in multiplex correlate very well with the binding events demonstrating the sensitivity of the NGS sequencing to quantify biomarkers in multiplex.

### Example 7

In this particular embodiment, the ability to extend off of target sequences within the tissue was tested using PE1.0-CHR7 (SEQ. ID. NO. 45) and PE1.0-CHR17 (SEQ. ID. NO. 46) probes. These experiments demonstrate polymerase extension without the added complexity of a photo-reactive step. The sequences were:

PE1.0-CHR7 (SEQ. ID. NO: 45):
5′-AATGATACGGCGACCACCGAGATCTACACTCTTTCCCTACACGACG
CTCTTCCGATCTTCTAGCCATTTGATGCCAACAGTAGAAAGGG-3′
PE1.0-CHR17 (SEQ. ID. NO: 46):
5′AATGATACGGCGACCACCGAGATCTACACTCTTTCCCTACACGACGCT
CTTCCGATCTCATTCAAATCCCCGAGTTGAACTTTCCTTTCAAAGTTCAC
GT-3′

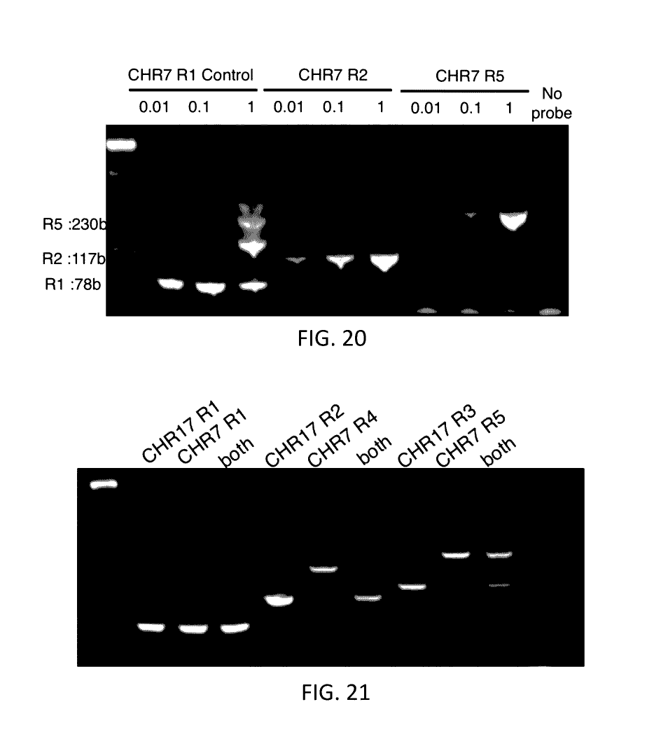

The probes were hybridized to tissue with increasing concentrations of 10 ng/ml, 0.1 μg/ml and 1 μg/ml per slide. Following deparaffinization and cell conditioning on the Benchmark XT, single or a cocktail of PE1.0-CHR7 and PE1.0-CHR17 probes, dNTPs and FastTaq DNA PoLymerase (Roche Applied Sciences) were added on the slides. Following hybridization and extension at 60° C. for 1 hour in a buffer comprising 50 mM Tris/HCl, 10 mM KCl, 5 mM (NH₄)₂SO₄, 2 mM MgCl₂, the probe sequences were collected off of the slides following a high temperature denaturation. The extended sequences were selectively amplified using a forward primer within the Illumina NGS adaptor sequence (SEQ ID NO: 26). The distance of the in situ polymerase extension was detected using reverse primers (SEQ ID NOS: 29-33) complementary to the regions further from the probe sequences (FIG. 18). Amplification was performed using the following thermal cycling conditions: 98° C. for 10 seconds, 60° C. for 10 s, 72° C. for 1 min (15-20 cycles), with modifications of the annealing temperatures based on the melting temperatures of the primer pairs. The products were analyzed using gel electrophoresis (Invitrogen 4% EZ Gel), and the results are provided in FIGS. 19 and 20.

As illustrated in FIGS. 19 and 20, as the concentration of each probe is increased, the obtained signal increases. FIGS. 19 and 20 also show signals obtained from the reverse primer extension, which indicates that the extension reaction is capable of producing extended products having high numbers of base pairs. As illustrated in FIG. 20, the method produced an extended product having about 230 base pairs. The results obtained from this particular embodiment illustrate the ability to extend primers that are hybridized to tissue without having to first isolate a section of the tissue sample. Rather, the primer can be added to a tissue sample, extended, amplified, and detected without ever having to destroy tissue context by physically excising and/or manipulating the tissue sample. Furthermore, it demonstrates the ability to change the detection length. The three extension lengths were selected to show an ability to extend to various lengths.

### Example 8

In this particular embodiment, CHR7 and CHR17 primers (SEQ ID NOs: 20 and 4, respectively) were hybridized to a tissue sample simultaneously with concentrations of 0.1 μg/ml each. The collected, extended sequences were amplified via duplex PCR using both of the extended CHR7 and CHR17 reverse primers (FIG. 21). This particular embodiment establishes that the disclosed methods are suitable for multiplexing assays. Again, these were done in the absence of a photo-reactive step to show robustness of the underlying extension approach.

### Example 9

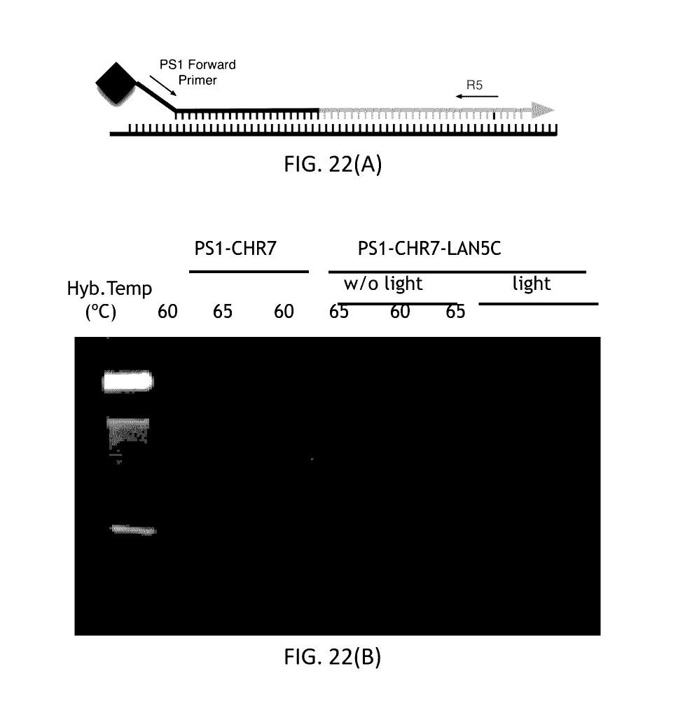

Referring now to FIGS. 22(A)-(B), FIG. 22(A) is a schematic showing that an approach by which the sequences could be purified using a biotin molecule at the 5′end (square). PS1 and CHR 7R5 (which is complementary to the region further from the probe sequence) were used to detect in situ polymerase extension. FIG. 22(B) is a photograph of a gel showing that subsequent PCR amplification, a 230 bp extended product was detected with the control probe Biotin-PS1-CHR7 with no caged thymidines (Lanes 1-2).

In this particular embodiment, light selective in situ polymerase extension using light-activated oligos (LANs) is demonstrated. In particular, light-activated oligonucleotides (LANs) (TriLink technologies) were used to demonstrate selective polymerase extension in tissue. LANs contain photolabile nucleobase modifications, nitropiperonyloxymethyl (NPOM), to one or more of the thymidines. The NPOM-caging group interrupts hybridization by sterically blocking Watson-Crick base pairing. Reference is made to Young et al., 2008 Light triggered polymerase chain reaction, Chem. Commun., 2008, 462-464, which is hereby incorporated by reference, herein, in its entirety for disclosure relate to in situ polymerase extension.

CHR7 probe was synthesized with a nonhuman priming sequence (PS1: SEQ. ID NO. 42) and a biotin molecule at the 5′-end and ‘caged’ thymidines distributed towards to 3′-end. The non-caged version of the probe was used as a positive control (FIG. 22(B), lanes 1-2).

The probes were hybridized to tissues on BenchMark XT instrument at 60° C. and 65° C. for 1 hour. After the hybridization, slides that will be illuminated by UV were taken off the instrument (FIG. 22(B), lanes 5-6) while the rest were kept dark (FIG. 22(B), lanes 3-4) in the instrument. The slides were treated with UV for 1 min with a UV lamp for slide illumination and put back in the instrument. The unbound material was removed from the slide with a stringency wash. The extension buffer containing Sequenase V2.0 DNA polymerase (Affymetrix, Inc) and dNTPs (Roche) were added on the slides to allow polymerase extension. The probe sequences were collected from the slides following a high temperature denaturation and purified with Dynabeads M-280 Streptavidin beads (Invitrogen). Purified sequences bound to the streptavidin beads were used as a template for detection PCR. Light selective in-situ polymerase extension was demonstrated by using PS1 as a forward primer and CHR7R5 reverse primer complementary to the regions 230 bp further from the probe sequence. The products were analyzed using gel electrophoresis (Invitrogen 4% EZ Gel).

While the probes from the slides without UV illumination failed to produce extended product due to the caged thymidines (Lanes 3-4), the probes on the UV illuminated slides were successfully extended and produced the expected size band (Lanes 5-6) at both hybridization temperatures. Primer and probe sequences used in this study:

PS1 (SEQ. ID NO: 42):
5′-TAACTTACGGAGTCGCTCTACG-3′
CHR7 (SEQ. ID NO: 43):
5′-TCTAGCCATTTGATGCCAACAGTAG AAAGGG-3′
Biotin-PS1-CHR7LAN5C (SEQ. ID NO: 44):
5′{Biotin}/TAACTTACGGAGTCGCTCTACGTCTAGCCA
T*T*T*GAT*GCCAACAGT*AGAAAGGG-3'
*T denotes positions of caged thymidines in caged versions of the probes.

### Example 10

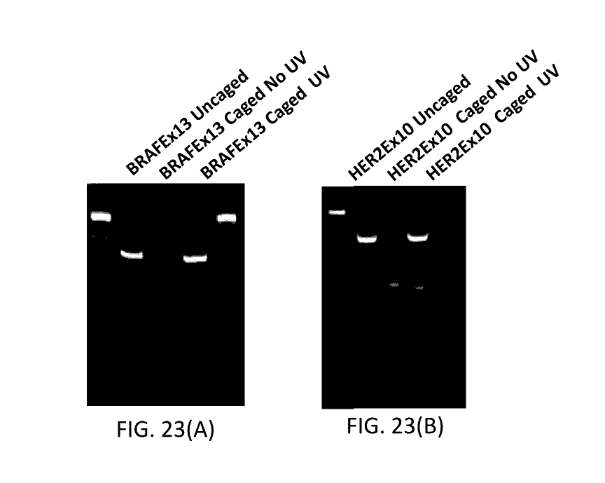

In this embodiment, we demonstrate photo-selective in situ polymerase extension of single genes. Referring now to FIG. 23(A)-(B), probes specific for BRAF Exon 13 (FIG. 23(A)) and for HER2 Exon 10 (FIG. 23(B)), was designed with and without caged thymidines. Both primers were hybridized and polymerase extended at 60° C. for 1 hour. The slide which was hybridized with the caged thymidines was treated with UV light, while the remainder was kept in the dark. Lane 1 (exclusive of the marker lanes) shows the control probe with no caged thymidines; thus, extension products are generated and detected as expected. Lane 2 shows the caged thymidine containing probe which had not been subjected to light. It is apparent from the gel that no extension occurred. Lane 3 shows the caged thymidine containing probe subjected to UV light and the extended product is detected at a level similar to that expected from the control probe (i.e. probe without caged thymidine). The light activated nucleotides were included at 5 different locations in both primers. Both exon primers were biotin labelled at the 5′ end, and extended primers were purified with streptavidin beads following denaturation at 98° C. for 3-4 min. The aliquots of the purified products were PCR amplified with the corresponding primer pairs and detected with Invitrogen 4% E-gel. The following probe sequences used in this study:

Biotin-HER2Exon10 (SEQ. ID NO: 47):
5′-Biotin-TAACTTACGGAGTC
GCTCTACGGCATGGAGCACT*T*GCGAG AGGT*GAGGGCAGT*T*A-3′
BiotinBRAFExon13 (SEQ. ID NO: 48):
5′-Biotin-TAACTTACGGAGTCGCTCTACGAG
TGGTGTGAGGGCTCCAGCTTGTAT*CACCAT*CT*AT*T*G-3′
*T denotes positions of caged thymidines in caged versions of the probes.

### Additional Exemplary Embodiments

The following additional embodiments are also specifically disclosed. This is not an exhaustive list.

1. A method for performing contextual molecular diagnostics, comprising:

contacting a sample with a probe comprising a photo-cleavable moiety, a specific binding moiety to a target, and a detection tag using conditions sufficient to facilitate binding of the specific binding moiety to a target in the sample;

removing probe that does not bind to the sample;

selecting a region of the sample for irradiation based on contextual information;

irradiating the selected region of the sample with light of a wavelength and an intensity sufficient to cleave the photo-cleavable moiety, thereby freeing the detection tag from the selected region of the sample; and

detecting the detection tag.

2. The method of embodiment 1, wherein

contacting includes contacting the sample with multiple probes having photo-cleavable moieties and specific binding moieties to multiple targets, the multiple different probes comprising unique detection tags and unique specific binding moieties and

detecting the detection tag includes detecting the multiple unique detection tags.

3. The method of embodiments 1 or 2, wherein selecting further comprises selecting additional regions; the method further comprising:

iteratively and separately irradiating the additional regions.

4. The embodiment of claim 3, the method further comprising iteratively depositing the detection tags from the additional regions in additional separate reaction vials.

5. The method of embodiment 4, wherein the additional separate reaction vials contain PCR primers with distinct ID sequences

6. The method of embodiment 5, further comprising appending the PCR primers to the detection tags.

7. The method of embodiment 4, further comprising iteratively and separately detecting the detection tag from the additional selected regions.

8. The method of embodiment 3, further comprising relating the detecting of the detection tag from the selected regions to the sample so as to create a molecular profile of the sample, wherein the molecular profile relates each selected area with the detection tags detected from the selected area across the sample.

9. The method of any of embodiments 1 to 8, wherein detecting the detection tag includes quantifying the number of targets in a sample.

10. The method of any of embodiments 1 to 9, wherein the specific binding moiety is selected from an amino acid, a peptide, a protein, an antibody, a nucleotide, an oligonucleotide, a polynucleotide, a primer, an aptamer, a binding variable region, a plasmid, DNA, RNA, miRNA, or combinations thereof.

11. The method of embodiment 1, wherein the method further comprises:

irradiating a second selected region of the sample to cleave the photo-cleavable moiety, thereby freeing the detection tag from the second selected region of the sample; and

detecting the detection tag that was bound to the target within the second selected region of the sample.

12. The method of any of embodiments 1 to 11, wherein the detection tag is a unique sequence identifier or a tag sequence.

13. The method of embodiment 12, wherein the unique sequence identifier is configured for analysis using an array.

14. The method of embodiment 12, comprising sequencing the unique sequence identifier or the tag.

15. The method of embodiment 12, wherein the unique sequence identifier is configured for sequencing using next generation sequencing.

16. The method of embodiment 12, wherein the unique sequence identifier comprises a reagent barcode, an adapter sequence, an alignment sequence, a subject index sequence, or combinations thereof.

17. The method of embodiment 12, wherein the unique sequence identifier or the tag sequence encodes information.

18. The method of embodiment 16, wherein the reagent barcode encodes lot data, manufacturing data, date information, source information, or combinations thereof.

19. The method of embodiment 16, wherein the reagent barcode comprises from about 6 nucleotides to about 200 nucleotides, the adapter sequence comprises from about 5 nucleotides to about 50 nucleotides, the alignment sequence comprises from about 3 nucleotides to at least about 20 nucleotides, and the subject index sequence comprises from at least 5 nucleotides to about 10 nucleotides.

20. The method of embodiment 12, wherein the tag sequence is capable of hybridizing with a template sequence to form a duplex.

21. The method of embodiment 12, wherein the tag sequence comprises one or more peptide nucleic acids, locked-nucleic acids, or combinations thereof.

22. The method of any of embodiments 1 to 11, wherein the detection tag further comprises a detectable label.

23. The method of embodiment 22, wherein the detectable label is a hapten.

24. The method of embodiment 23, wherein the method further includes purifying the detection tag using by binding the hapten to an anti-hapten anti-body.

25. The method of any of embodiments 1 to 24, wherein the probe further comprises at least one linker that links two probe components.

26. The method of any of embodiments 1 to 25, wherein the photo-cleavable moiety is cleaved by light having a wavelength ranging from about 200 nm to about 400 nm.

27. The method of any of embodiments 1 to 25, wherein the photo-cleavable moiety is cleaved by light having a wavelength ranging from about 400 nm to about 790 nm.

28. The method of any of embodiments 1 to 25, wherein the photo-cleavable moiety is selected from a photo-cleavable phosphoramidite, photo-cleavable biotin, or photo-cleavable amine.

29. The method of any of embodiments 1 to 28, wherein the sample is irradiated with light indirectly.

30. The method of any of claims 1 to 29, wherein the sample is irradiated with light by using a digital micromirror device.

31. The method of any of embodiments 1 to 28, wherein the sample is irradiated with light directly.

32. The method of embodiment 29, wherein the sample is irradiated with light indirectly by using a liquid crystal display.

33. The method of any of embodiments 1 to 32, wherein the sample is irradiated with light for a time sufficient to cleave the photo-cleavable moiety.

34. The method of any of embodiments 1 to 33, wherein the sample is irradiated for a time period ranging from about 1 second to about 60 seconds.

35. The method of any of embodiments 1 to 34, wherein the light is produced by a light source selected from a laser, an LED, or a lamp.

36. The method of any of embodiments 1 to 35, wherein the conditions sufficient to bind the specific binding moiety of the probe to the sample are suitable for inducing hybridization and/or chemical coupling of the probe to the sample.

37. The method of any of embodiments 1 to 36, wherein removing probe that does not bind to the sample includes washing the sample with a suitable washing solution.

38. The method of any of embodiments 1 to 37, wherein selecting the region of the sample comprises manually marking one or more portions of the sample or digitally marking one or more portions of the sample.

39. The method of any of embodiments 1 to 38, wherein the contextual information used for selecting is created using a combination of one or more of primary staining, secondary staining, fluorescence imaging, phase contrast imaging, morphological sample characteristics imaging, image analysis, digital image analysis, cell selection, or grouping selection.

40. The method of any of embodiments 1 to 39, wherein the method further comprises contacting the sample with a visually detectable reagent and selecting includes the contextual information provided by the visually detectable reagent.

41. The method of embodiment 40, wherein the detectable reagent includes a fluorescent or chromogenic reagent.

42. The method of any of embodiments 1 to 41, wherein detection of the detection tag indicates that the target is present within the region.

43. The method of embodiment 1, wherein detection of the detection tag further comprises sequencing the detection tag using next generation sequencing, analyzing the detection tag using PCR, or analyzing the detection tag using an array platform.

44. The method of embodiment 1, further comprising contacting the sample with a second probe comprising a second specific binding moiety, a second photo-cleavable moiety, and a second detection tag.

45. The method of embodiment 44, wherein the photo-cleavable moiety and the second photo-cleavable moiety are the same.

46. The method of embodiment 44 or 45, wherein the detection tag and the second detection tag are different and

the specific binding moiety and the second specific binding moiety are different.

47. The method of any of embodiments 44 to 46, further comprising relating the detecting of the detection tag from the selected regions to the sample so as to create a molecular profile of the sample, wherein the molecular profile relates each selected area with the detection tags detected from the selected area across the sample and a unique specific binding moiety

48. The method of any of embodiments 44 to 47 wherein contacting the sample with the probe occurs simultaneously with contacting the sample with the second probe.

49. The method of any of embodiments 1 to 48, wherein selecting further comprises selecting a control region; the method further comprising:

separately irradiating the control region and

separately detecting whether the detection tag is present in the control region.

50. A method for performing time-elapsed contextual molecular diagnostics, comprising:

performing the method of embodiment 1 on a first selected region,

archiving the sample,

removing the sample from archival, and

performing the method of claim 1 on a second selected region.

51. A method for performing contextual molecular diagnostics, the method comprising:

contacting a sample with a probe comprising a photo-reactive moiety, a specific binding moiety, and a detection tag using conditions sufficient to facilitate binding of the specific binding moiety to a target in the sample;

removing probe that does not bind to the sample;

selecting a region of the sample for irradiation based on contextual information;

irradiating the selected region of the sample with light of a wavelength and an intensity sufficient to cause the photo-reactive moiety to react, thereby freeing the detection sequence for further reaction;

exposing the tissue sample to an enzyme that acts on the detection sequence to effect a change; and

detecting the change.

52. The method of embodiment 51, wherein the detection tag is an extendable primer, the photo-reactive moiety is a photo-cleavable blocking group, the enzyme is a polymerase, and the change is an extension of a primer sequence in the presence of dATP, dCTP, dTTP, and dGTP.

53. The method of embodiments 51 or 52, further comprising ligating adjacent detection tag after irradiating the selected region.

54. The method of embodiment 51, wherein the photo-cleavable moiety includes a caged-ATP molecule.

55. The method of embodiment 54, wherein the caged-ATP molecule is converted to an ATP molecule capable of modifying a probe present on the sample with a phosphate group.

56. The method of claim 55, further comprising modifying the phosphate group with a detectable label, a member of a specific binding pair, or a combination thereof.

57. The method of claim 56, further comprising detecting the detectable label or isolating the probe using the member of the specific binding pair.

58. The method of any of claims 51 to 57, wherein contacting includes multiple probes having specific binding moieties to multiple targets, the multiple different probes comprising unique detection tags and unique specific binding moieties and detecting the detection tag includes detecting the multiple unique detection tags.

59. The method of any of claims 51 to 58, wherein selecting further comprises selecting additional regions; the method further comprising:

iteratively and separately irradiating the additional regions and

iteratively and separately detecting the detection tag from the additional selected regions.

60. The method of claim 59, further comprising relating the detecting of the detection tags from the selected regions to the sample so as to create a molecular profile of the sample, wherein the molecular profile relates each selected area with the detection tags detected from the selected area across the sample.

61. The method of any of claims 51 to 60, wherein detecting the detection tag includes quantifying the number of targets in a sample.

62. The method of any of claims 51 to 61, wherein the specific binding moiety is selected from an amino acid, a peptide, a protein, an antibody, a nucleotide, an oligonucleotide, a polynucleotide, a primer, an aptamer, a binding variable region, a plasmid, DNA, RNA, miRNA, or combinations thereof.

63. The method of claim 51, wherein the method further comprises:

irradiating a second selected region of the sample to cause the photo-reactive moiety to react, thereby freeing the detection sequence for further reaction from the second selected region of the sample; and

detecting the detection tag that was bound to the target within the second selected region of the sample.

64. The method of claim 51, wherein the detection tag is a unique sequence identifier or a tag sequence.

65. The method of claim 51, wherein selecting the region of the sample comprises manually marking one or more portions of the sample or digitally marking one or more portions of the sample.

66. The method of any of claims 51 to 65, wherein the contextual information used for selecting is created using a combination of one or more of primary staining, secondary staining, fluorescence imaging, phase contrast imaging, morphological sample characteristics imaging, image analysis, digital image analysis, cell selection, or grouping selection.

67. The method of any of claims 51 to 65, wherein the method further comprises contacting the sample with a visually detectable reagent and selecting includes the contextual information provided by the visually detectable reagent.

68. The method of any of claims 51 to 67, wherein detection of the detection tag indicates that the target is present within the region.

69. The method of claim 51, wherein detection of the detection tag further comprises sequencing the detection tag using next generation sequencing, analyzing the detection tag using PCR, or analyzing the detection tag using an array platform.

70. The method according to claim 51, further comprising contacting the sample with a second probe comprising a second specific binding moiety, a second photo-reactive moiety, and a second detection tag.

71. The method according to claim 70, wherein the photo-reactive moiety and the second photo-reactive moiety are the same, the detection tag and the second detection tag are different, and the specific binding moiety and the second specific binding moiety are different.

72. A system for performing contextual diagnostics using a photo-activated probe, comprising:

a slide configured to accept a sample;

a vessel containing the photo-activated probe;

a slide imaging device configured to receive the slide, and

a light source configured to selectively irradiate the sample.

73. The system of claim 72, wherein the photo-activated probe comprises a specific binding moiety, a photo-activated moiety, and a detection tag, wherein the photo-activated probe is configured to be detectable upon selective irradiation.

74. The system of claim 72 or 73, wherein the slide is a single specimen slide, an array slide, or a microarray slide.

75. The system of any of claims 72 to 74, wherein the light source is selected from a laser, an LED, or a lamp.

76. The system of any of claims 72 to 75, wherein the slide imaging device is configured to capture a sample image.

77. The system of claim 76, the system further comprising

a digital screen in communication with the imaging device for receiving and displaying the sample image from the imaging device; and

a selection device in communication with the digital screen for marking a selected portion of the sample image for analysis.

78. The system of claim 77, wherein the light source is coupled to the selection device for irradiating the selected portion.

79. The system of any of claims 76 to 78, wherein the slide imaging device is a digital camera or a video camera.

80. The system of any of claims 73 to 79, further comprising: a dispensing instrument configured to dispense an elution buffer onto the slide thereby facilitating isolation of the detection tag; and

a recovery instrument configured to recover the detection tag.

81. The system of any of claims 73 to 80, further comprising a PCR instrument configured to accept and amplify the detection tag.

82. The system of any of claims 73 to 81, further comprising:

a sequencing instrument configured to receive and sequence the detection tag; and
a computer configured to translate information generated from the sequencing instrument to the digital screen, thereby correlating the detection tag to the selected region from which it was cleaved.

83. The system of any of claims 73 to 80, further comprising an array platform comprising a modified surface configured to accept and bind the detection tag.

84. A kit for performing contextual diagnostics, comprising a probe reagent solution comprising a specific binding moiety, a photo-cleavable moiety, and a detection tag.

85. A method for performing contextual molecular diagnostics, the method comprising:

contacting a sample with a probe comprising a specific binding moiety, using conditions sufficient to facilitate binding of the specific binding moiety to a target in the sample;

removing probe that does not bind to the sample;

contacting a sample with a photo-reactive moiety;

selecting a region of the sample for irradiation based on contextual information;

irradiating the selected region of the sample with light of a wavelength and an intensity sufficient to cause the photo-reactive moiety to react;

exposing the tissue sample to an enzyme that acts on the detection oligonucleotide to effect a change; and

detecting the change.

86. The method according to claim 85, wherein the detection tag is an extendable primer, the photo-reactive moiety is one or more caged deoxynucleoside triphosphate molecule(s), taken from the group of deoxynucleoside triphosphates including dATP, dCTP, dGTP, dTTP, and dUTP or derivatives thereof, with exclusion of the corresponding uncaged nucleoside triphosphates.

87. The method of claim 86, wherein the caged-nucleoside triphophate molecule(s) is/are converted to nucleoside triphosphate molecule(s) capable of being incorporated by a polymerase to extend the extendable primer.

### SEQUENCE LISTING

The nucleic and amino acid sequences provided herein are shown using standard letter abbreviations for nucleotide bases, and three letter code for amino acids, as defined in 37 C.F.R. §1.822. Only one strand of each nucleic acid sequence is shown, but the complementary strand is understood as included by any reference to the displayed strand.

SEQ ID NO: 1 is an exemplary sequence for a unique
sequence identifier.
ATGGTATCCTTACATCGTCATTTATC
SEQ ID NO: 2 is another exemplary sequence for a
unique sequence identifier.
CCTCTCTATGGGCAGTCGGTGATTCCTATTCAGATCACGATGGTATCCTT
ACATCTGAGTCGGAGACACGCAGGGATGAGATGG
SEQ ID NO: 3 is yet another exemplary sequence for
a unique sequence identifier.
CCTCTCTATGGGCAGTCGGTGATTCCTATTCAGATGGTATCCTTACATCT
GAGTCGGAGACACGCAGGGATGAGATGG
SEQ ID NO: 4 is an exemplary sequence for an
oligo/polynucleotide specific binding moiety.
CATTCAAATCCCCGAGTTGAACTTTCCTTTCAAAGTTCACGT
SEQ ID NO: 5 is another exemplary sequence for an
oligo/polynucleotide specific binding moiety.
CGGGAGGCGGAGGTTGCAGTGAGCC
SEQ ID NO: 6 is an exemplary primer sequence for
use with a probe disclosed herein.
GTAAAACGACGGCCAGT
SEQ ID NO: 7 is another exemplary primer sequence
for use with a probe disclosed herein.
CAGGAAACAGCTATGAC
SEQ ID NO: 8 is a Her1 amino acid sequence
(NCBI Reference Sequence No. NP_005219.2).
MRPSGTAGAALLALLAALCPASRALEEKKVCQGTSNKLTQLGTFEDHFLS
LQRMENNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHIPPLDPQELDILKTVKE
ITGFLLIQAWPENRIDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRREQERELVEPLIPSGEAPNQ
ALLRILKETEFKKIKVLGSGAFGIVYKGLWIPEGEKVKIPVAIKELREAT
SPKANKEILDEAYVMASVDNPHVCRLLGICLTSTVQLITQLMPFGCLLDY
VREHKDNIGSQYLLNWCVQIAKGMNYLEDRRLVHRDLAARNVLVKTPQHV
KITDFGLAKLLGAEEKEYHAEGGKVPIKWMALESILHRIYTHQSDVWSYG
VTVWELMTFGSKPYDGIPASEISSILEKGERLPQPPICTIDVYMIMVKCW
MIDADSRPKFRELIIEFSKMARDPQRYLVIQGDERMHLPSPTDSNFYRAL
MDEEDMDDVVDADEYLIPQQGFFSSPSTSRTPLLSSLSATSNNSTVACID
RNGLQSCPIKEDSFLQRYSSDPTGALTEDSIDDTFLPVPEYINQSVPKRP
AGSVQNPVYHNQPLNPAPSRDPHYQDPHSTAVGNPEYLNTVQPTCVNSTF
DSPAHWAQKGSHQISLDNPDYQQDFFPKEAKPNGIFKGSTAENAEYLRVA
PQSSEFIGA
SEQ ID NO: 9 is an exemplary cDNA sequence
encoding Her1 (NCBI Reference Sequence No.
NM_005228.3).
CCCCGGCGCAGCGCGGCCGCAGCAGCCTCCGCCCCCCGCACGGTGTGAGC
GCCCGACGCGGCCGAGGCGGCCGGAGTCCCGAGCTAGCCCCGGCGGCCGC
CGCCGCCCAGACCGGACGACAGGCCACCTCGTCGGCGTCCGCCCGAGTCC
CCGCCTCGCCGCCAACGCCACAACCACCGCGCACGGCCCCCTGACTCCGT
CCAGTATTGATCGGGAGAGCCGGAGCGAGCTCTTCGGGGAGCAGCGATGC
GACCCTCCGGGACGGCCGGGGCAGCGCTCCTGGCGCTGCTGGCTGCGCTC
TGCCCGGCGAGTCGGGCTCTGGAGGAAAAGAAAGTTTGCCAAGGCACGAG
TAACAAGCTCACGCAGTTGGGCACTTTTGAAGATCATTTTCTCAGCCTCC
AGAGGATGTTCAATAACTGTGAGGTGGTCCTTGGGAATTTGGAAATTACC
TATGTGCAGAGGAATTATGATCTTTCCTTCTTAAAGACCATCCAGGAGGT
GGCTGGTTATGTCCTCATTGCCCTCAACACAGTGGAGCGAATTCCTTTGG
AAAACCTGCAGATCATCAGAGGAAATATGTACTACGAAAATTCCTATGCC
TTAGCAGTCTTATCTAACTATGATGCAAATAAAACCGGACTGAAGGAGCT
GCCCATGAGAAATTTACAGGAAATCCTGCATGGCGCCGTGCGGTTCAGCA
ACAACCCTGCCCTGTGCAACGTGGAGAGCATCCAGTGGCGGGACATAGTC
AGCAGTGACTTTCTCAGCAACATGTCGATGGACTTCCAGAACCACCTGGG
CAGCTGCCAAAAGTGTGATCCAAGCTGTCCCAATGGGAGCTGCTGGGGTG
CAGGAGAGGAGAACTGCCAGAAACTGACCAAAATCATCTGTGCCCAGCAG
TGCTCCGGGCGCTGCCGTGGCAAGTCCCCCAGTGACTGCTGCCACAACCA
GTGTGCTGCAGGCTGCACAGGCCCCCGGGAGAGCGACTGCCTGGTCTGCC
GCAAATTCCGAGACGAAGCCACGTGCAAGGACACCTGCCCCCCACTCATG
CTCTACAACCCCACCACGTACCAGATGGATGTGAACCCCGAGGGCAAATA
CAGCTTTGGTGCCACCTGCGTGAAGAAGTGTCCCCGTAATTATGTGGTGA
CAGATCACGGCTCGTGCGTCCGAGCCTGTGGGGCCGACAGCTATGAGATG
GAGGAAGACGGCGTCCGCAAGTGTAAGAAGTGCGAAGGGCCTTGCCGCAA
AGTGTGTAACGGAATAGGTATTGGTGAATTTAAAGACTCACTCTCCATAA
ATGCTACGAATATTAAACACTTCAAAAACTGCACCTCCATCAGTGGCGAT
CTCCACATCCTGCCGGTGGCATTTAGGGGTGACTCCTTCACACATACTCC
TCCTCTGGATCCACAGGAACTGGATATTCTGAAAACCGTAAAGGAAATCA
CAGGGTTTTTGCTGATTCAGGCTTGGCCTGAAAACAGGACGGACCTCCAT
GCCTTTGAGAACCTAGAAATCATACGCGGCAGGACCAAGCAACATGGTCA
GTTTTCTCTTGCAGTCGTCAGCCTGAACATAACATCCTTGGGATTACGCT
CCCTCAAGGAGATAAGTGATGGAGATGTGATAATTTCAGGAAACAAAAAT
TTGTGCTATGCAAATACAATAAACTGGAAAAAACTGTTTGGGACCTCCGG
TCAGAAAACCAAAATTATAAGCAACAGAGGTGAAAACAGCTGCAAGGCCA
CAGGCCAGGTCTGCCATGCCTTGTGCTCCCCCGAGGGCTGCTGGGGCCCG
GAGCCCAGGGACTGCGTCTCTTGCCGGAATGTCAGCCGAGGCAGGGAATG
CGTGGACAAGTGCAACCTTCTGGAGGGTGAGCCAAGGGAGTTTGTGGAGA
ACTCTGAGTGCATACAGTGCCACCCAGAGTGCCTGCCTCAGGCCATGAAC
ATCACCTGCACAGGACGGGGACCAGACAACTGTATCCAGTGTGCCCACTA
CATTGACGGCCCCCACTGCGTCAAGACCTGCCCGGCAGGAGTCATGGGAG
AAAACAACACCCTGGTCTGGAAGTACGCAGACGCCGGCCATGTGTGCCAC
CTGTGCCATCCAAACTGCACCTACGGATGCACTGGGCCAGGTCTTGAAGG
CTGTCCAACGAATGGGCCTAAGATCCCGTCCATCGCCACTGGGATGGTGG
GGGCCCTCCTCTTGCTGCTGGTGGTGGCCCTGGGGATCGGCCTCTTCATG
CGAAGGCGCCACATCGTTCGGAAGCGCACGCTGCGGAGGCTGCTGCAGGA
GAGGGAGCTTGTGGAGCCTCTTACACCCAGTGGAGAAGCTCCCAACCAAG
CTCTCTTGAGGATCTTGAAGGAAACTGAATTCAAAAAGATCAAAGTGCTG
GGCTCCGGTGCGTTCGGCACGGTGTATAAGGGACTCTGGATCCCAGAAGG
TGAGAAAGTTAAAATTCCCGTCGCTATCAAGGAATTAAGAGAAGCAACAT
CTCCGAAAGCCAACAAGGAAATCCTCGATGAAGCCTACGTGATGGCCAGC
GTGGACAACCCCCACGTGTGCCGCCTGCTGGGCATCTGCCTCACCTCCAC
CGTGCAGCTCATCACGCAGCTCATGCCCTTCGGCTGCCTCCTGGACTATG
TCCGGGAACACAAAGACAATATTGGCTCCCAGTACCTGCTCAACTGGTGT
GTGCAGATCGCAAAGGGCATGAACTACTTGGAGGACCGTCGCTTGGTGCA
CCGCGACCTGGCAGCCAGGAACGTACTGGTGAAAACACCGCAGCATGTCA
AGATCACAGATTTTGGGCTGGCCAAACTGCTGGGTGCGGAAGAGAAAGAA
TACCATGCAGAAGGAGGCAAAGTGCCTATCAAGTGGATGGCATTGGAATC
AATTTTACACAGAATCTATACCCACCAGAGTGATGTCTGGAGCTACGGGG
TGACCGTTTGGGAGTTGATGACCTTTGGATCCAAGCCATATGACGGAATC
CCTGCCAGCGAGATCTCCTCCATCCTGGAGAAAGGAGAACGCCTCCCTCA
GCCACCCATATGTACCATCGATGTCTACATGATCATGGTCAAGTGCTGGA
TGATAGACGCAGATAGTCGCCCAAAGTTCCGTGAGTTGATCATCGAATTC
TCCAAAATGGCCCGAGACCCCCAGCGCTACCTTGTCATTCAGGGGGATGA
AAGAATGCATTTGCCAAGTCCTACAGACTCCAACTTCTACCGTGCCCTGA
TGGATGAAGAAGACATGGACGACGTGGTGGATGCCGACGAGTACCTCATC
CCACAGCAGGGCTTCTTCAGCAGCCCCTCCACGTCACGGACTCCCCTCCT
GAGCTCTCTGAGTGCAACCAGCAACAATTCCACCGTGGCTTGCATTGATA
GAAATGGGCTGCAAAGCTGTCCCATCAAGGAAGACAGCTTCTTGCAGCGA
TACAGCTCAGACCCCACAGGCGCCTTGACTGAGGACAGCATAGACGACAC
CTTCCTCCCAGTGCCTGAATACATAAACCAGTCCGTTCCCAAAAGGCCCG
CTGGCTCTGTGCAGAATCCTGTCTATCACAATCAGCCTCTGAACCCCGCG
CCCAGCAGAGACCCACACTACCAGGACCCCCACAGCACTGCAGTGGGCAA
CCCCGAGTATCTCAACACTGTCCAGCCCACCTGTGTCAACAGCACATTCG
ACAGCCCTGCCCACTGGGCCCAGAAAGGCAGCCACCAAATTAGCCTGGAC
AACCCTGACTACCAGCAGGACTTCTTTCCCAAGGAAGCCAAGCCAAATGG
CATCTTTAAGGGCTCCACAGCTGAAAATGCAGAATACCTAAGGGTCGCGC
CACAAAGCAGTGAATTTATTGGAGCATGACCACGGAGGATAGTATGAGCC
CTAAAAATCCAGACTCTTTCGATACCCAGGACCAAGCCACAGCAGGTCCT
CCATCCCAACAGCCATGCCCGCATTAGCTCTTAGACCCACAGACTGGTTT
TGCAACGTTTACACCGACTAGCCAGGAAGTACTTCCACCTCGGGCACATT
TTGGGAAGTTGCATTCCTTTGTCTTCAAACTGTGAAGCATTTACAGAAAC
GCATCCAGCAAGAATATTGTCCCTTTGAGCAGAAATTTATCTTTCAAAGA
GGTATATTTGAAAAAAAAAAAAAGTATATGTGAGGATTTTTATTGATTGG
GGATCTTGGAGTTTTTCATTGTCGCTATTGATTTTTACTTCAATGGGCTC
TTCCAACAAGGAAGAAGCTTGCTGGTAGCACTTGCTACCCTGAGTTCATC
CAGGCCCAACTGTGAGCAAGGAGCACAAGCCACAAGTCTTCCAGAGGATG
CTTGATTCCAGTGGTTCTGCTTCAAGGCTTCCACTGCAAAACACTAAAGA
TCCAAGAAGGCCTTCATGGCCCCAGCAGGCCGGATCGGTACTGTATCAAG
TCATGGCAGGTACAGTAGGATAAGCCACTCTGTCCCTTCCTGGGCAAAGA
AGAAACGGAGGGGATGGAATTCTTCCTTAGACTTACTTTTGTAAAAATGT
CCCCACGGTACTTACTCCCCACTGATGGACCAGTGGTTTCCAGTCATGAG
CGTTAGACTGACTTGTTTGTCTTCCATTCCATTGTTTTGAAACTCAGTAT
GCTGCCCCTGTCTTGCTGTCATGAAATCAGCAAGAGAGGATGACACATCA
AATAATAACTCGGATTCCAGCCCACATTGGATTCATCAGCATTTGGACCA
ATAGCCCACAGCTGAGAATGTGGAATACCTAAGGATAGCACCGCTTTTGT
TCTCGCAAAAACGTATCTCCTAATTTGAGGCTCAGATGAAATGCATCAGG
TCCTTTGGGGCATAGATCAGAAGACTACAAAAATGAAGCTGCTCTGAAAT
CTCCTTTAGCCATCACCCCAACCCCCCAAAATTAGTTTGTGTTACTTATG
GAAGATAGTTTTCTCCTTTTACTTCACTTCAAAAGCTTTTTACTCAAAGA
GTATATGTTCCCTCCAGGTCAGCTGCCCCCAAACCCCCTCCTTACGCTTT
GTCACACAAAAAGTGTCTCTGCCTTGAGTCATCTATTCAAGCACTTACAG
CTCTGGCCACAACAGGGCATTTTACAGGTGCGAATGACAGTAGCATTATG
AGTAGTGTGGAATTCAGGTAGTAAATATGAAACTAGGGTTTGAAATTGAT
AATGCTTTCACAACATTTGCAGATGTTTTAGAAGGAAAAAAGTTCCTTCC
TAAAATAATTTCTCTACAATTGGAAGATTGGAAGATTCAGCTAGTTAGGA
GCCCACCTTTTTTCCTAATCTGTGTGTGCCCTGTAACCTGACTGGTTAAC
AGCAGTCCTTTGTAAACAGTGTTTTAAACTCTCCTAGTCAATATCCACCC
CATCCAATTTATCAAGGAAGAAATGGTTCAGAAAATATTTTCAGCCTACA
GTTATGTTCAGTCACACACACATACAAAATGTTCCTTTTGCTTTTAAAGT
AATTTTTGACTCCCAGATCAGTCAGAGCCCCTACAGCATTGTTAAGAAAG
TATTTGATTTTTGTCTCAATGAAAATAAAACTATATTCATTTCCACTCTA
AAAAAAAAAAAAAAAA
SEQ ID NO: 10 is a Her2 amino acid sequence (NCBI
Reference Sequence No. NP_004439.2).
MELAALCRWGLLLALLPPGAASTQVCTGTDMKLRLPASPETHLDMLRHLY
QGCQWQGNLELTYLPTNASLSFLQDIQEVQGYVLIAHNQVRQVPLQRLRI
VRGTQLFEDNYALAVLDNGDPLNNTTPVTGASPGGLRELQLRSLTEILKG
GVLIQRNPQLCYQDTILWKDIFHKNNQLALTLIDTNRSRACHPCSPMCKG
SRCWGESSEDCQSLTRTVCAGGCARCKGPLPTDCCHEQCAAGCTGPKHSD
CLACLHFNHSGICELHCPALVTYNTDTFESMPNPEGRYTFGASCVTACPY
NYLSTDVGSCTLVCPLHNQEVTAEDGTQRCEKCSKPCARVCYGLGMEHLR
EVRAVTSANIQEFAGCKKIFGSLAFLPESFDGDPASNTAPLQPEQLQVFE
TLEEITGYLYISAWPDSLPDLSVFQNLQVIRGRILHNGAYSLTLQGLGIS
WLGLRSLRELGSGLALIHHNTHLCFVHTVPWDQLFRNPHQALLHTANRPE
DECVGEGLACHQLCARGHCWGPGPTQCVNCSQFLRGQECVEECRVLQGLP
REYVNARHCLPCHPECQPQNGSVTCFGPEADQCVACAHYKDPPFCVARCP
SGVKPDLSYMPIWKFPDEEGACQPCPINCTHSCVDLDDKGCPAEQRASPL
TSIISAWGILLWVLGWFGILIKRRQQKIRKYTMRRLLQETELVEPLTPSG
AMPNQAQMRILKETELRKVKVLGSGAFGTVYKGIWIPDGENVKIPVAIKV
LRENTSPKANKEILDEAYVMAGVGSPYVSRLLGICLTSTVQLVTQLMPYG
CLLDHVRENRGRLGSQDLLNWCMQIAKGMSYLEDVRLVHRDLAARNVLVK
SPNHVKITDFGLARLLDIDETEYHADGGKVPIKWMALESILRRRFTHQSD
VWSYGVTVWELMTFGAKPYDGIPAREIPDLLEKGERLPQPPICTIDVYMI
MVKCWMIDSECRPRFRELVSEFSRMARDPQRFWIQNEDLGPASPLDSTFY
RSLLEDDDMGDLVDAEEYLVPQQGFFCPDPAPGAGGMVHHRHRSSSTRSG
GGDLTLGLEPSEEEAPRSPLAPSEGAGSDVFDGDLGMGAAKGLQSLPTHD
PSPLQRYSEDPTVPLPSETDGYVAPLTCSPQPEYVNQPDVRPQPPSPREG
PLPAARPAGATLERPKTLSPGKNGWKDVFAFGGAVENPEYLTPQGGAAPQ
PHPPPAFSPAFDNLYYWDQDPPERGAPPSTFKGTPTAENPEYLGLDVPV
SEQ ID NO: 11 is an exemplary cDNA sequence
encoding Her2 (NCBI Reference Sequence No.
NM_004448.2).
GGAGGAGGTGGAGGAGGAGGGCTGCTTGAGGAAGTATAAGAATGAAGTTG
TGAAGCTGAGATTCCCCTCCATTGGGACCGGAGAAACCAGGGGAGCCCCC
CGGGCAGCCGCGCGCCCCTTCCCACGGGGCCCTTTACTGCGCCGCGCGCC
CGGCCCCCACCCCTCGCAGCACCCCGCGCCCCGCGCCCTCCCAGCCGGGT
CCAGCCGGAGCCATGGGGCCGGAGCCGCAGTGAGCACCATGGAGCTGGCG
GCCTTGTGCCGCTGGGGGCTCCTCCTCGCCCTCTTGCCCCCCGGAGCCGC
GAGCACCCAAGTGTGCACCGGCACAGACATGAAGCTGCGGCTCCCTGCCA
GTCCCGAGACCCACCTGGACATGCTCCGCCACCTCTACCAGGGCTGCCAG
GTGGTGCAGGGAAACCTGGAACTCACCTACCTGCCCACCAATGCCAGCCT
GTCCTTCCTGCAGGATATCCAGGAGGTGCAGGGCTACGTGCTCATCGCTC
ACAACCAAGTGAGGCAGGTCCCACTGCAGAGGCTGCGGATTGTGCGAGGC
ACCCAGCTCTTTGAGGACAACTATGCCCTGGCCGTGCTAGACAATGGAGA
CCCGCTGAACAATACCACCCCTGTCACAGGGGCCTCCCCAGGAGGCCTGC
GGGAGCTGCAGCTTCGAAGCCTCACAGAGATCTTGAAAGGAGGGGTCTTG
ATCCAGCGGAACCCCCAGCTCTGCTACCAGGACACGATTTTGTGGAAGGA
CATCTTCCACAAGAACAACCAGCTGGCTCTCACACTGATAGACACCAACC
GCTCTCGGGCCTGCCACCCCTGTTCTCCGATGTGTAAGGGCTCCCGCTGC
TGGGGAGAGAGTTCTGAGGATTGTCAGAGCCTGACGCGCACTGTCTGTGC
CGGTGGCTGTGCCCGCTGCAAGGGGCCACTGCCCACTGACTGCTGCCATG
AGCAGTGTGCTGCCGGCTGCACGGGCCCCAAGCACTCTGACTGCCTGGCC
TGCCTCCACTTCAACCACAGTGGCATCTGTGAGCTGCACTGCCCAGCCCT
GGTCACCTACAACACAGACACGTTTGAGTCCATGCCCAATCCCGAGGGCC
GGTATACATTCGGCGCCAGCTGTGTGACTGCCTGTCCCTACAACTACCTT
TCTACGGACGTGGGATCCTGCACCCTCGTCTGCCCCCTGCACAACCAAGA
GGTGACAGCAGAGGATGGAACACAGCGGTGTGAGAAGTGCAGCAAGCCCT
GTGCCCGAGTGTGCTATGGTCTGGGCATGGAGCACTTGCGAGAGGTGAGG
GCAGTTACCAGTGCCAATATCCAGGAGTTTGCTGGCTGCAAGAAGATCTT
TGGGAGCCTGGCATTTCTGCCGGAGAGCTTTGATGGGGACCCAGCCTCCA
ACACTGCCCCGCTCCAGCCAGAGCAGCTCCAAGTGTTTGAGACTCTGGAA
GAGATCACAGGTTACCTATACATCTCAGCATGGCCGGACAGCCTGCCTGA
CCTCAGCGTCTTCCAGAACCTGCAAGTAATCCGGGGACGAATTCTGCACA
ATGGCGCCTACTCGCTGACCCTGCAAGGGCTGGGCATCAGCTGGCTGGGG
CTGCGCTCACTGAGGGAACTGGGCAGTGGACTGGCCCTCATCCACCATAA
CACCCACCTCTGCTTCGTGCACACGGTGCCCTGGGACCAGCTCTTTCGGA
ACCCGCACCAAGCTCTGCTCCACACTGCCAACCGGCCAGAGGACGAGTGT
GTGGGCGAGGGCCTGGCCTGCCACCAGCTGTGCGCCCGAGGGCACTGCTG
GGGTCCAGGGCCCACCCAGTGTGTCAACTGCAGCCAGTTCCTTCGGGGCC
AGGAGTGCGTGGAGGAATGCCGAGTACTGCAGGGGCTCCCCAGGGAGTAT
GTGAATGCCAGGCACTGTTTGCCGTGCCACCCTGAGTGTCAGCCCCAGAA
TGGCTCAGTGACCTGTTTTGGACCGGAGGCTGACCAGTGTGTGGCCTGTG
CCCACTATAAGGACCCTCCCTTCTGCGTGGCCCGCTGCCCCAGCGGTGTG
AAACCTGACCTCTCCTACATGCCCATCTGGAAGTTTCCAGATGAGGAGGG
CGCATGCCAGCCTTGCCCCATCAACTGCACCCACTCCTGTGTGGACCTGG
ATGACAAGGGCTGCCCCGCCGAGCAGAGAGCCAGCCCTCTGACGTCCATC
ATCTCTGCGGTGGTTGGCATTCTGCTGGTCGTGGTCTTGGGGGTGGTCTT
TGGGATCCTCATCAAGCGACGGCAGCAGAAGATCCGGAAGTACACGATGC
GGAGACTGCTGCAGGAAACGGAGCTGGTGGAGCCGCTGACACCTAGCGGA
GCGATGCCCAACCAGGCGCAGATGCGGATCCTGAAAGAGACGGAGCTGAG
GAAGGTGAAGGTGCTTGGATCTGGCGCTTTTGGCACAGTCTACAAGGGCA
TCTGGATCCCTGATGGGGAGAATGTGAAAATTCCAGTGGCCATCAAAGTG
TTGAGGGAAAACACATCCCCCAAAGCCAACAAAGAAATCTTAGACGAAGC
ATACGTGATGGCTGGTGTGGGCTCCCCATATGTCTCCCGCCTTCTGGGCA
TCTGCCTGACATCCACGGTGCAGCTGGTGACACAGCTTATGCCCTATGGC
TGCCTCTTAGACCATGTCCGGGAAAACCGCGGACGCCTGGGCTCCCAGGA
CCTGCTGAACTGGTGTATGCAGATTGCCAAGGGGATGAGCTACCTGGAGG
ATGTGCGGCTCGTACACAGGGACTTGGCCGCTCGGAACGTGCTGGTCAAG
AGTCCCAACCATGTCAAAATTACAGACTTCGGGCTGGCTCGGCTGCTGGA
CATTGACGAGACAGAGTACCATGCAGATGGGGGCAAGGTGCCCATCAAGT
GGATGGCGCTGGAGTCCATTCTCCGCCGGCGGTTCACCCACCAGAGTGAT
GTGTGGAGTTATGGTGTGACTGTGTGGGAGCTGATGACTTTTGGGGCCAA
ACCTTACGATGGGATCCCAGCCCGGGAGATCCCTGACCTGCTGGAAAAGG
GGGAGCGGCTGCCCCAGCCCCCCATCTGCACCATTGATGTCTACATGATC
ATGGTCAAATGTTGGATGATTGACTCTGAATGTCGGCCAAGATTCCGGGA
GTTGGTGTCTGAATTCTCCCGCATGGCCAGGGACCCCCAGCGCTTTGTGG
TCATCCAGAATGAGGACTTGGGCCCAGCCAGTCCCTTGGACAGCACCTTC
TACCGCTCACTGCTGGAGGACGATGACATGGGGGACCTGGTGGATGCTGA
GGAGTATCTGGTACCCCAGCAGGGCTTCTTCTGTCCAGACCCTGCCCCGG
GCGCTGGGGGCATGGTCCACCACAGGCACCGCAGCTCATCTACCAGGAGT
GGCGGTGGGGACCTGACACTAGGGCTGGAGCCCTCTGAAGAGGAGGCCCC
CAGGTCTCCACTGGCACCCTCCGAAGGGGCTGGCTCCGATGTATTTGATG
GTGACCTGGGAATGGGGGCAGCCAAGGGGCTGCAAAGCCTCCCCACACAT
GACCCCAGCCCTCTACAGCGGTACAGTGAGGACCCCACAGTACCCCTGCC
CTCTGAGACTGATGGCTACGTTGCCCCCCTGACCTGCAGCCCCCAGCCTG
AATATGTGAACCAGCCAGATGTTCGGCCCCAGCCCCCTTCGCCCCGAGAG
GGCCCTCTGCCTGCTGCCCGACCTGCTGGTGCCACTCTGGAAAGGCCCAA
GACTCTCTCCCCAGGGAAGAATGGGGTCGTCAAAGACGTTTTTGCCTTTG
GGGGTGCCGTGGAGAACCCCGAGTACTTGACACCCCAGGGAGGAGCTGCC
CCTCAGCCCCACCCTCCTCCTGCCTTCAGCCCAGCCTTCGACAACCTCTA
TTACTGGGACCAGGACCCACCAGAGCGGGGGGCTCCACCCAGCACCTTCA
AAGGGACACCTACGGCAGAGAACCCAGAGTACCTGGGTCTGGACGTGCCA
GTGTGAACCAGAAGGCCAAGTCCGCAGAAGCCCTGATGTGTCCTCAGGGA
GCAGGGAAGGCCTGACTTCTGCTGGCATCAAGAGGTGGGAGGGCCCTCCG
ACCACTTCCAGGGGAACCTGCCATGCCAGGAACCTGTCCTAAGGAACCTT
CCTTCCTGCTTGAGTTCCCAGATGGCTGGAAGGGGTCCAGCCTCGTTGGA
AGAGGAACAGCACTGGGGAGTCTTTGTGGATTCTGAGGCCCTGCCCAATG
AGACTCTAGGGTCCAGTGGATGCCACAGCCCAGCTTGGCCCTTTCCTTCC
AGATCCTGGGTACTGAAAGCCTTAGGGAAGCTGGCCTGAGAGGGGAAGCG
GCCCTAAGGGAGTGTCTAAGAACAAAAGCGACCCATTCAGAGACTGTCCC
TGAAACCTAGTACTGCCCCCCATGAGGAAGGAACAGCAATGGTGTCAGTA
TCCAGGCTTTGTACAGAGTGCTTTTCTGTTTAGTTTTTACTTTTTTTGTT
TTGTTTTTTTAAAGATGAAATAAAGACCCAGGGGGAGAATGGGTGTTGTA
TGGGGAGGCAAGTGTGGGGGGTCCTTCTCCACACCCACTTTGTCCATTTG
CAAATATATTTTGGAAAACAGCTA
SEQ ID NO: 12 is a Her3 amino acid sequence (NCBI
Reference Sequence No. NP_001973.2).
MRANDALQVLGLLFSLARGSEVGNSQAVCPGTLNGLSVTGDAENQYQTLY
KLYERCEVVMGNLEIVLTGHNADLSFLQWIREVTGYVLVAMNEFSTLPLP
NLRVVRGTQVYDGKFAIFVMLNYNTNSSHALRQLRLTQLTEILSGGVYIE
KNDKLCHMDTIDWRDIVRDRDAEIVVKDNGRSCPPCHEVCKGRCWGPGSE
DCQTLTKTICAPQCNGHCFGPNPNQCCHDECAGGCSGPQDTDCFACRHFN
DSGACVPRCPQPLVYNKLTFQLEPNPHTKYQYGGVCVASCPHNFVVDQTS
CVRACPPDKMEVDKNGLKMCEPCGGLCPKACEGTGSGSRFQTVDSSNIDG
FVNCTKILGNLDFLITGLNGDPWHKIPALDPEKLNVFRTVREITGYLNIQ
SWPPHMHNFSVFSNLTTIGGRSLYNRGFSLLIMKNLNVTSLGFRSLKEIS
AGRIYISANRQLCYHHSLNWTKVLRGPTEERLDIKHNRPRRDCVAEGKVC
DPLCSSGGCWGPGPGQCLSCRNYSRGGVCVTHCNFLNGEPREFAHEAECF
SCHPECQPMEGTATCNGSGSDTCAQCAHFRDGPHCVSSCPHGVLGAKGPI
YKYPDVQNECRPCHENCTQGCKGPELQDCLGQTLVLIGKTHLTMALTVIA
GLVVIFMMLGGTFLYWRGRRIQNKRAMRRYLERGESIEPLDPSEKANKVL
ARIFKETELRKLKVLGSGVFGTVHKGVWIPEGESIKIPVCIKVIEDKSGR
QSFQAVTDHMLAIGSLDHAHIVRLLGLCPGSSLQLVTQYLPLGSLLDHVR
QHRGALGPQLLLNWGVQIAKGMYYLEEHGMVHRNLAARNVLLKSPSQVQV
ADFGVADLLPPDDKQLLYSEAKTPIKWMALESIHFGKYTHQSDVWSYGVT
VWELMTFGAEPYAGLRLAEVPDLLEKGERLAQPQICTIDVYMVMVKCWMI
DENIRPTFKELANEFTRMARDPPRYLVIKRESGPGIAPGPEPHGLTNKKL
EEVELEPELDLDLDLEAEEDNLATTTLGSALSLPVGTLNRPRGSQSLLSP
SSGYMPMNQGNLGESCQESAVSGSSERCPRPVSLHPMPRGCLASESSEGH
VIGSEAELQEKVSMCRSRSRSRSPRPRGDSAYHSQRHSLLTPVTPLSPPG
LEEEDVNGYVMPDTHLKGTPSSREGTLSSVGLSSVLGTEEEDEDEEYEYM
NRRRRHSPPHPPRPSSLEELGYEYMDVGSDLSASLGSTQSCPLHPVPIMP
TAGTTPDEDYEYMNRQRDGGGPGGDYAAMGACPASEQGYEEMRAFQGPGH
QAPHVHYARLKTLRSLEATDSAFDNPDYWHSRLFPKANAQRT
SEQ ID NO: 13 is an exemplary cDNA sequence
encoding Her3 (NCBI Reference Sequence No.
NM_001982.3).
ACTCCAGCCTCGCGCGGGAGGGGGCGCGGCCGTGACTCACCCCCTTCCCT
CTGCGTTCCTCCCTCCCTCTCTCTCTCTCTCTCACACACACACACCCCTC
CCCTGCCATCCCTCCCCGGACTCCGGCTCCGGCTCCGATTGCAATTTGCA
ACCTCCGCTGCCGTCGCCGCAGCAGCCACCAATTCGCCAGCGGTTCAGGT
GGCTCTTGCCTCGATGTCCTAGCCTAGGGGCCCCCGGGCCGGACTTGGCT
GGGCTCCCTTCACCCTCTGCGGAGTCATGAGGGCGAACGACGCTCTGCAG
GTGCTGGGCTTGCTTTTCAGCCTGGCCCGGGGCTCCGAGGTGGGCAACTC
TCAGGCAGTGTGTCCTGGGACTCTGAATGGCCTGAGTGTGACCGGCGATG
CTGAGAACCAATACCAGACACTGTACAAGCTCTACGAGAGGTGTGAGGTG
GTGATGGGGAACCTTGAGATTGTGCTCACGGGACACAATGCCGACCTCTC
CTTCCTGCAGTGGATTCGAGAAGTGACAGGCTATGTCCTCGTGGCCATGA
ATGAATTCTCTACTCTACCATTGCCCAACCTCCGCGTGGTGCGAGGGACC
CAGGTCTACGATGGGAAGTTTGCCATCTTCGTCATGTTGAACTATAACAC
CAACTCCAGCCACGCTCTGCGCCAGCTCCGCTTGACTCAGCTCACCGAGA
TTCTGTCAGGGGGTGTTTATATTGAGAAGAACGATAAGCTTTGTCACATG
GACACAATTGACTGGAGGGACATCGTGAGGGACCGAGATGCTGAGATAGT
GGTGAAGGACAATGGCAGAAGCTGTCCCCCCTGTCATGAGGTTTGCAAGG
GGCGATGCTGGGGTCCTGGATCAGAAGACTGCCAGACATTGACCAAGACC
ATCTGTGCTCCTCAGTGTAATGGTCACTGCTTTGGGCCCAACCCCAACCA
GTGCTGCCATGATGAGTGTGCCGGGGGCTGCTCAGGCCCTCAGGACACAG
ACTGCTTTGCCTGCCGGCACTTCAATGACAGTGGAGCCTGTGTACCTCGC
TGTCCACAGCCTCTTGTCTACAACAAGCTAACTTTCCAGCTGGAACCCAA
TCCCCACACCAAGTATCAGTATGGAGGAGTTTGTGTAGCCAGCTGTCCCC
ATAACTTTGTGGTGGATCAAACATCCTGTGTCAGGGCCTGTCCTCCTGAC
AAGATGGAAGTAGATAAAAATGGGCTCAAGATGTGTGAGCCTTGTGGGGG
ACTATGTCCCAAAGCCTGTGAGGGAACAGGCTCTGGGAGCCGCTTCCAGA
CTGTGGACTCGAGCAACATTGATGGATTTGTGAACTGCACCAAGATCCTG
GGCAACCTGGACTTTCTGATCACCGGCCTCAATGGAGACCCCTGGCACAA
GATCCCTGCCCTGGACCCAGAGAAGCTCAATGTCTTCCGGACAGTACGGG
AGATCACAGGTTACCTGAACATCCAGTCCTGGCCGCCCCACATGCACAAC
TTCAGTGTTTTTTCCAATTTGACAACCATTGGAGGCAGAAGCCTCTACAA
CCGGGGCTTCTCATTGTTGATCATGAAGAACTTGAATGTCACATCTCTGG
GCTTCCGATCCCTGAAGGAAATTAGTGCTGGGCGTATCTATATAAGTGCC
AATAGGCAGCTCTGCTACCACCACTCTTTGAACTGGACCAAGGTGCTTCG
GGGGCCTACGGAAGAGCGACTAGACATCAAGCATAATCGGCCGCGCAGAG
ACTGCGTGGCAGAGGGCAAAGTGTGTGACCCACTGTGCTCCTCTGGGGGA
TGCTGGGGCCCAGGCCCTGGTCAGTGCTTGTCCTGTCGAAATTATAGCCG
AGGAGGTGTCTGTGTGACCCACTGCAACTTTCTGAATGGGGAGCCTCGAG
AATTTGCCCATGAGGCCGAATGCTTCTCCTGCCACCCGGAATGCCAACCC
ATGGAGGGCACTGCCACATGCAATGGCTCGGGCTCTGATACTTGTGCTCA
ATGTGCCCATTTTCGAGATGGGCCCCACTGTGTGAGCAGCTGCCCCCATG
GAGTCCTAGGTGCCAAGGGCCCAATCTACAAGTACCCAGATGTTCAGAAT
GAATGTCGGCCCTGCCATGAGAACTGCACCCAGGGGTGTAAAGGACCAGA
GCTTCAAGACTGTTTAGGACAAACACTGGTGCTGATCGGCAAAACCCATC
TGACAATGGCTTTGACAGTGATAGCAGGATTGGTAGTGATTTTCATGATG
CTGGGCGGCACTTTTCTCTACTGGCGTGGGCGCCGGATTCAGAATAAAAG
GGCTATGAGGCGATACTTGGAACGGGGTGAGAGCATAGAGCCTCTGGACC
CCAGTGAGAAGGCTAACAAAGTCTTGGCCAGAATCTTCAAAGAGACAGAG
CTAAGGAAGCTTAAAGTGCTTGGCTCGGGTGTCTTTGGAACTGTGCACAA
AGGAGTGTGGATCCCTGAGGGTGAATCAATCAAGATTCCAGTCTGCATTA
AAGTCATTGAGGACAAGAGTGGACGGCAGAGTTTTCAAGCTGTGACAGAT
CATATGCTGGCCATTGGCAGCCTGGACCATGCCCACATTGTAAGGCTGCT
GGGACTATGCCCAGGGTCATCTCTGCAGCTTGTCACTCAATATTTGCCTC
TGGGTTCTCTGCTGGATCATGTGAGACAACACCGGGGGGCACTGGGGCCA
CAGCTGCTGCTCAACTGGGGAGTACAAATTGCCAAGGGAATGTACTACCT
TGAGGAACATGGTATGGTGCATAGAAACCTGGCTGCCCGAAACGTGCTAC
TCAAGTCACCCAGTCAGGTTCAGGTGGCAGATTTTGGTGTGGCTGACCTG
CTGCCTCCTGATGATAAGCAGCTGCTATACAGTGAGGCCAAGACTCCAAT
TAAGTGGATGGCCCTTGAGAGTATCCACTTTGGGAAATACACACACCAGA
GTGATGTCTGGAGCTATGGTGTGACAGTTTGGGAGTTGATGACCTTCGGG
GCAGAGCCCTATGCAGGGCTACGATTGGCTGAAGTACCAGACCTGCTAGA
GAAGGGGGAGCGGTTGGCACAGCCCCAGATCTGCACAATTGATGTCTACA
TGGTGATGGTCAAGTGTTGGATGATTGATGAGAACATTCGCCCAACCTTT
AAAGAACTAGCCAATGAGTTCACCAGGATGGCCCGAGACCCACCACGGTA
TCTGGTCATAAAGAGAGAGAGTGGGCCTGGAATAGCCCCTGGGCCAGAGC
CCCATGGTCTGACAAACAAGAAGCTAGAGGAAGTAGAGCTGGAGCCAGAA
CTAGACCTAGACCTAGACTTGGAAGCAGAGGAGGACAACCTGGCAACCAC
CACACTGGGCTCCGCCCTCAGCCTACCAGTTGGAACACTTAATCGGCCAC
GTGGGAGCCAGAGCCTTTTAAGTCCATCATCTGGATACATGCCCATGAAC
CAGGGTAATCTTGGGGAGTCTTGCCAGGAGTCTGCAGTTTCTGGGAGCAG
TGAACGGTGCCCCCGTCCAGTCTCTCTACACCCAATGCCACGGGGATGCC
TGGCATCAGAGTCATCAGAGGGGCATGTAACAGGCTCTGAGGCTGAGCTC
CAGGAGAAAGTGTCAATGTGTAGGAGCCGGAGCAGGAGCCGGAGCCCACG
GCCACGCGGAGATAGCGCCTACCATTCCCAGCGCCACAGTCTGCTGACTC
CTGTTACCCCACTCTCCCCACCCGGGTTAGAGGAAGAGGATGTCAACGGT
TATGTCATGCCAGATACACACCTCAAAGGTACTCCCTCCTCCCGGGAAGG
CACCCTTTCTTCAGTGGGTCTCAGTTCTGTCCTGGGTACTGAAGAAGAAG
ATGAAGATGAGGAGTATGAATACATGAACCGGAGGAGAAGGCACAGTCCA
CCTCATCCCCCTAGGCCAAGTTCCCTTGAGGAGCTGGGTTATGAGTACAT
GGATGTGGGGTCAGACCTCAGTGCCTCTCTGGGCAGCACACAGAGTTGCC
CACTCCACCCTGTACCCATCATGCCCACTGCAGGCACAACTCCAGATGAA
GACTATGAATATATGAATCGGCAACGAGATGGAGGTGGTCCTGGGGGTGA
TTATGCAGCCATGGGGGCCTGCCCAGCATCTGAGCAAGGGTATGAAGAGA
TGAGAGCHTTCAGGGGCCTGGACATCAGGCCCCCCATGTCCATTATGCCC
GCCTAAAAACTCTACGTAGCTTAGAGGCTACAGACTCTGCCTTTGATAAC
CCTGATTACTGGCATAGCAGGCTTTTCCCCAAGGCTAATGCCCAGAGAAC
GTAACTCCTGCTCCCTGTGGCACTCAGGGAGCATTTAATGGCAGCTAGTG
CCTTTAGAGGGTACCGTCTTCTCCCTATTCCCTCTCTCTCCCAGGTCCCA
GCCCCTTTTCCCCAGTCCCAGACAATTCCATTCAATCTTTGGAGGCTTTT
AAACATTTTGACACAAAATTCTTATGGTATGTAGCCAGCTGTGCACTTTC
TTCTCTTTCCCAACCCCAGGAAAGGTTTTCCTTATTTTGTGTGCTTTCCC
AGTCCCATTCCTCAGCTTCTTCACAGGCACTCCTGGAGATATGAAGGATT
ACTCTCCATATCCCTTCCTCTCAGGCTCTTGACTACTTGGAACTAGGCTC
TTATGTGTGCCTTTGTTTCCCATCAGACTGTCAAGAAGAGGAAAGGGAGG
AAACCTAGCAGAGGAAAGTGTAATTTTGGTTTATGACTCTTAACCCCCTA
GAAAGACAGAAGCTTAAAATCTGTGAAGAAAGAGGTTAGGAGTAGATATT
GATTACTATCATAATTCAGCACTTAACTATGAGCCAGGCATCATACTAAA
CTTCACCTACATTATCTCACTTAGTCCTTTATCATCCTTAAAACAATTCT
GTGACATACATATTATCTCATTTTACACAAAGGGAAGTCGGGCATGGTGG
CTCATGCCTGTAATCTCAGCACTTTGGGAGGCTGAGGCAGAAGGATTACC
TGAGGCAAGGAGTTTGAGACCAGCTTAGCCAACATAGTAAGACCCCCATC
TCTTTAAAAAAAAAAAAAAAAAAAAAAAAAAAACTTTAGAACTGGGTGCA
GTGGCTCATGCCTGTAATCCCAGCCAGCACTTTGGGAGGCTGAGATGGGA
AGATCACTTGAGCCCAGAATTAGAGATAAGCCTATGGAAACATAGCAAGA
CACTGTCTCTACAGGGGAAAAAAAAAAAAGAAACTGAGCCTTAAAGAGAT
GAAATAAATTAAGCAGTAGATCCAGGATGCAAAATCCTCCCAATTCCTGT
GCATGTGCTCTTATTGTAAGGTGCCAAGAAAAACTGATTTAAGTTACAGC
CCTTGTTTAAGGGGCACTGTTTCTTGTTTTTGCACTGAATCAAGTCTAAC
CCCAACAGCCACATCCTCCTATACCTAGACATCTCATCTCAGGAAGTGGT
GGTGGGGGTAGTCAGAAGGAAAAATAACTGGACATCTTTGTGTAAACCAT
AATCCACATGTGCCGTAAATGATCTTCACTCCTTATCCGAGGGCAAATTC
ACAAGGATCCCCAAGATCCACTTTTAGAAGCCATTCTCATCCAGCAGTGA
GAAGCTTCCAGGTAGGACAGAAAAAAGATCCAGCTTCAGCTGCACACCTC
TGTCCCCTTGGATGGGGAACTAAGGGAAAACGTCTGTTGTATCACTGAAG
TTTTTTGTTTTGTTTTTATACGTGTCTGAATAAAAATGCCAAAGTTTTTT
TTCAGCAAAAAAAA
SEQ ID NO: 14 is a Her4 amino acid sequence
(Genbank Accession No. AAI43750).
MKPATGLWVWVSLLVAAGTVQPSDSQSVCAGTENKLSSLSDLEQQYRALR
KYYENCEVVMGNLEITSIEHNRDLSFLRSVREVTGYVLVALNQFRYLPLE
NLRIIRGTKLYEDRYALAIFLNYRKDGNFGLQELGLKNLTEILNGGVYVD
QNKFLCYADTIHWQDIVRNFWPSNLTLVSTNGSSGCGRCHKSCTGRCWGP
TENHCQTLTRTVCAEQCDGRCYGPYVSDCCHRECAGGCSGPKDTDCFACM
NFNDSGACWQCPQTFVYNPTTFQLEHNFNAKYTYGAFCVKKCPHNFWDSS
SCVRACPSSKMEVEENGIKMCKPCTDICPKACDGIGTGSLMSAQTVDSSN
IDKFINCTKINGNLIFLVTGIHGDPYNAIEAIDPEKLNVFRTVREITGFL
NIQSWPPNMTDFSVFSNLVTIGGRVLYSGLSLLILKQQGITSLQFQSLKE
ISAGNIYITDNSNLCYYHTINWTTLFSTINQRIVIRDNRKAENCTAEGMV
CNHLCSSDGCWGPGPDQCLSCRRFSRGRICIESCNLYDGEFREFENGSIC
VECDPQCEKMEDGLLTCHGPGPDNCTKCSHFKDGPNCVEKCPDGLQGANS
FIFKYADPDRECHPCHPNCTQGCIGSSIEDCIGLMDRTPLIAAGVIGGLF
ILVIVGLTFAVYVRRKSIKKKRALRRFLETELVEPLTPSGTAPNQAQLRI
LKETELKRVKVLGSGAFGTVYKGIWVPEGETVKIPVAIKILNETTGPKAN
VEFMDEALIMASMDHPHLVRLLGVCLSPTIQLVTQLMPHGCLLEYVHEHK
DNIGSQLLLNWCVQIAKGMMYLEERRLVHRDLAARNVLVKSPNHVKITDF
GLARLLEGDEKEYNADGGKMPIKWMALECIHYRKFTHQSDVWSYGVTIWE
LMTFGGKPYDGIPTREIPDLLEKGERLPQPPICTIDVYMVMVKCWMIDAD
SRPKFKELAAEFSRMARDPQRYLVIQGDDRMKLPSPNDSKFFQNLLDEED
LEDMMDAEEYLVPQAFNIPPPIYTSRARIDSNRNQFVYRDGGFAAEQGVS
VPYRAPTSTIPEAPVAQGATAEIFDDSCCNGTLRKPVAPHVQEDSSTQRY
SADPTVFAPERSPRGELDEEGYMTPMRDKPKQEYLNPVEENPFVSRRKNG
DLQALDNPEYHNASNGPPKAEDEYVNEPLYLNTFANTLGKAEYLKNNILS
MPEKAKKAFDNPDYWNHSLPPRSTLQHPDYLQEYSTKYFYKQNGRIRPIV
AENPEYLSEFSLKPGTVLPPPPYRHRNTVV
SEQ ID NO: 15 is an exemplary cDNA sequence
encoding Her4 (GenBank Accession No. BC143749.1).
CAGCACGGGATCTGAGACTTCCAAAAAATGAAGCCGGCGACAGGACTTTG
GGTCTGGGTGAGCCTTCTCGTGGCGGCGGGGACCGTCCAGCCCAGCGATT
CTCAGTCAGTGTGTGCAGGAACGGAGAATAAACTGAGCTCTCTCTCTGAC
CTGGAACAGCAGTACCGAGCCTTGCGCAAGTACTATGAAAACTGTGAGGT
TGTCATGGGCAACCTGGAGATAACCAGCATTGAGCACAACCGGGACCTCT
CCTTCCTGCGGTCTGTTCGAGAAGTCACAGGCTACGTGTTAGTGGCTCTT
AATCAGTTTCGTTACCTGCCTCTGGAGAATTTACGCATTATTCGTGGGAC
AAAACTTTATGAGGATCGATATGCCTTGGCAATATTTTTAAACTACAGAA
AAGATGGAAACTTTGGACTTCAAGAACTTGGATTAAAGAACTTGACAGAA
ATCCTAAATGGTGGAGTCTATGTAGACCAGAACAAATTCCTTTGTTATGC
AGACACCATTCATTGGCAAGATATTGTTCGGAACCCATGGCCTTCCAACT
TGACTCTTGTGTCAACAAATGGTAGTTCAGGATGTGGACGTTGCCATAAG
TCCTGTACTGGCCGTTGCTGGGGACCCACAGAAAATCATTGCCAGACTTT
GACAAGGACGGTGTGTGCAGAACAATGTGACGGCAGATGCTACGGACCTT
ACGTCAGTGACTGCTGCCATCGAGAATGTGCTGGAGGCTGCTCAGGACCT
AAGGACACAGACTGCTTTGCCTGCATGAATTTCAATGACAGTGGAGCATG
TGTTACTCAGTGTCCCCAAACCTTTGTCTACAATCCAACCACCTTTCAAC
TGGAGCACAATTTCAATGCAAAGTACACATATGGAGCATTCTGTGTCAAG
AAATGTCCACATAACTTTGTGGTAGATTCCAGTTCTTGTGTGCGTGCCTG
CCCTAGTTCCAAGATGGAAGTAGAAGAAAATGGGATTAAAATGTGTAAAC
CTTGCACTGACATTTGCCCAAAAGCTTGTGATGGCATTGGCACAGGATCA
TTGATGTCAGCTCAGACTGTGGATTCCAGTAACATTGACAAATTCATAAA
CTGTACCAAGATCAATGGGAATTTGATCTTTCTAGTCACTGGTATTCATG
GGGACCCTTACAATGCAATTGAAGCCATAGACCCAGAGAAACTGAACGTC
TTTCGGACAGTCAGAGAGATAACAGGTTTCCTGAACATACAGTCATGGCC
ACCAAACATGACTGACTTCAGTGTTTTTTCTAACCTGGTGACCATTGGTG
GAAGAGTACTCTATAGTGGCCTGTCCTTGCTTATCCTCAAGCAACAGGGC
ATCACCTCTCTACAGTTCCAGTCCCTGAAGGAAATCAGCGCAGGAAACAT
CTATATTACTGACAACAGCAACCTGTGTTATTATCATACCATTAACTGGA
CAACACTCTTCAGCACAATCAACCAGAGAATAGTAATCCGGGACAACAGA
AAAGCTGAAAATTGTACTGCTGAAGGAATGGTGTGCAACCATCTGTGTTC
CAGTGATGGCTGTTGGGGACCTGGGCCAGACCAATGTCTGTCGTGTCGCC
GCTTCAGTAGAGGAAGGATCTGCATAGAGTCTTGTAACCTCTATGATGGT
GAATTTCGGGAGTTTGAGAATGGCTCCATCTGTGTGGAGTGTGACCCCCA
GTGTGAGAAGATGGAAGATGGCCTCCTCACATGCCATGGACCGGGTCCTG
ACAACTGTACAAAGTGCTCTCATTTTAAAGATGGCCCAAACTGTGTGGAA
AAATGTCCAGATGGCTTACAGGGGGCAAACAGTTTCATTTTCAAGTATGC
TGATCCAGATCGGGAGTGCCACCCATGCCATCCAAACTGCACCCAAGGGT
GCATAGGCTCAAGTATTGAAGACTGCATCGGCCTGATGGATAGAACTCCC
CTGATTGCAGCTGGAGTAATTGGTGGGCTCTTCATTCTGGTCATTGTGGG
TCTGACATTTGCTGTTTATGTTAGAAGGAAGAGCATCAAAAAGAAAAGAG
CCTTGAGAAGATTCTTGGAAACAGAGTTGGTGGAACCATTAACTCCCAGT
GGCACAGCACCCAATCAAGCTCAACTTCGTATTTTGAAAGAAACTGAGCT
GAAGAGGGTAAAAGTCCTTGGCTCAGGTGCTTTTGGAACGGTTTATAAAG
GTATTTGGGTACCTGAAGGAGAAACTGTGAAGATTCCTGTGGCTATTAAG
ATTCTTAATGAGACAACTGGTCCCAAGGCAAATGTGGAGTTCATGGATGA
AGCTCTGATCATGGCAAGTATGGATCATCCACACCTAGTCCGGTTGCTGG
GTGTGTGTCTGAGCCCAACCATCCAGCTGGTTACTCAACTTATGCCCCAT
GGCTGCCTGTTGGAGTATGTCCACGAGCACAAGGATAACATTGGATCACA
ACTGCTGCTTAACTGGTGTGTCCAGATAGCTAAGGGAATGATGTACCTGG
AAGAAAGACGACTCGTTCATCGGGATTTGGCAGCCCGTAATGTCTTAGTG
AAATCTCCAAACCATGTGAAAATCACAGATTTTGGGCTAGCCAGACTCTT
GGAAGGAGATGAAAAAGAGTACAATGCTGATGGAGGAGATGCCAATTAAA
TGGATGGCTCTGGAGTGTATACATTACAGGAAATTCACCCATCAGTGACG
TTTGGAGCTATGGAGTTACTATATGGGAACTGATGACCTTTGGAGGAAAA
CTATGATGGAATTCCAACGCGAGAAATCCCTGATTTATTAGAGAAAGGAG
AACGTTTGCCTCAGCCTCCCATCTGCACTATTGACGTTTACATGGTCATG
GTCAAATGTTGGATGATTGATGCTGACAGTAGACCTAAATTTAAGGAACT
GGCTGCTGAGTTTTCAAGGATGGCTCGAGACCCTCAAAGATACCTAGTTA
TTCAGGGTGATGATCGTATGAAGCTTCCCAGTCCAAATGACAGCAAGTTC
TTTCAGAATCTCTTGGATGAAGAGGATTTGGAAGATATGATGGATGCTGA
GGAGTACTTGGTCCCTCAGGCTTTCAACATCCCACCTCCCATCTATACTT
CCAGAGCAAGAATTGACTCGAATAGGAACCAGTTTGTGTACCGAGATGGA
GGTTTTGCTGCTGAACAAGGAGTGTCTGTGCCCTACAGAGCCCCAACTAG
CACAATTCCAGAAGCTCCTGTGGCACAGGGTGCTACTGCTGAGATTTTTG
ATGACTCCTGCTGTAATGGCACCCTACGCAAGCCAGTGGCACCCCATGTC
CAAGAGGACAGTAGCACCCAGAGGTACAGTGCTGACCCCACCGTGTTTGC
CCCAGAACGGAGCCCACGAGGAGAGCTGGATGAGGAAGGTTACATGACTC
CTATGCGAGACAAACCCAAACAAGAATACCTGAATCCAGTGGAGGAGAAC
CCTTTTGTTTCTCGGAGAAAAAATGGAGACCTTCAAGCATTGGATAATCC
CGAATATCACAATGCATCCAATGGTCCACCCAAGGCCGAGGATGAGTATG
TGAATGAGCCACTGTACCTCAACACCTTTGCCAACACCTTGGGAAAAGCT
GAGTACCTGAAGAACAACATACTGTCAATGCCAGAGAAGGCCAAGAAAGC
GTTTGACAACCCTGACTACTGGAACCACAGCCTGCCACCTCGGAGCACCC
TTCAGCACCCAGACTACCTGCAGGAGTACAGCACAAAATATTTTTATAAA
CAGAATGGGCGGATCCGGCCTATTGTGGCAGAGAATCCTGAATACCTCTC
TGAGTTCTCCCTGAAGCCAGGCACTGTGCTGCCGCCTCCACCTTACAGAC
ACCGGAATACTGTGGTGTAAGCTCAGTTGTGGTTTTTTAGGTGGAGAGAC
ACACCTGCTCCAATTTCCCCACCCC
SEQ ID NO: 16 is an Estrogen Receptor amino acid
sequence (NCBI Reference Sequence No.
NP_000116.2).
MTMTLHTKASGMALLHQIQGNELEPLNRPQLKIPLERPLGEVYLDSSKPA
VYNYPEGAAYEFNAAAAANAQVYGQTGLPYGPGSEAAAFGSNGLGGFPPL
NSVSPSPLMLLHPPPQLSPFLQPHGQQVPYYLENEPSGYTVREAGPPAFY
RPNSDNRRQGGRERLASTNDKGSMAMESAKETRYCAVCNDYASGYHYGVW
SCEGCKAFFKRSIQGHNDYMCPATNQCTIDKNRRKSCQACRLRKCYEVGM
MKGGIRKDRRGGRMLKHKRQRDDGEGRGEVGSAGDMRAANLWPSPLMIKR
SKKNSLALSLTADQMVSALLDAEPPILYSEYDPTRPFSEASMMGLLTNLA
DRELVHMINWAKRVPGFVDLTLHDQVHLLECAWLEILMIGLVWRSMEHPG
KLLFAPNLLLDRNQGKCVEGMVEIFDMLLATSSRFRMMNLQGEEFVCLKS
IILLNSGVYTFLSSTLKSLEEKDHIHRVLDKITDTLIHLMAKAGLTLQQQ
HQRLAQLLLILSHIRHMSNKGMEHLYSMKCKNVVPLYDLLLEMLDAHRLH
APTSRGGASVEETDQSHLATAGSTSSHSLQKYYITGEAEGFPATV
SEQ ID NO: 17 is an exemplary cDNA sequence
encoding an Estrogen Receptor (NCBI Reference
Sequence No. NM_000125.3).
AGGAGCTGGCGGAGGGCGTTCGTCCTGGGACTGCACTTGCTCCCGTCGGG
TCGCCCGGCTTCACCGGACCCGCAGGCTCCCGGGGCAGGGCCGGGGCCAG
AGCTCGCGTGTCGGCGGGACATGCGCTGCGTCGCCTCTAACCTCGGGCTG
TGCTCTTTTTCCAGGTGGCCCGCCGGTTTCTGAGCCTTCTGCCCTGCGGG
GACACGGTCTGCACCCTGCCCGCGGCCACGGACCATGACCATGACCCTCC
ACACCAAAGCATCTGGGATGGCCCTACTGCATCAGATCCAAGGGAACGAG
CTGGAGCCCCTGAACCGTCCGCAGCTCAAGATCCCCCTGGAGCGGCCCCT
GGGCGAGGTGTACCTGGACAGCAGCAAGCCCGCCGTGTACAACTACCCCG
AGGGCGCCGCCTACGAGTTCAACGCCGCGGCCGCCGCCAACGCGCAGGTC
TACGGTCAGACCGGCCTCCCCTACGGCCCCGGGTCTGAGGCTGCGGCGTT
CGGCTCCAACGGCCTGGGGGGTTTCCCCCCACTCAACAGCGTGTCTCCGA
GCCCGCTGATGCTACTGCACCCGCCGCCGCAGCTGTCGCCTTTCCTGCAG
CCCCACGGCCAGCAGGTGCCCTACTACCTGGAGAACGAGCCCAGCGGCTA
CACGGTGCGCGAGGCCGGCCCGCCGGCATTCTACAGGCCAAATTCAGATA
ATCGACGCCAGGGTGGCAGAGAAAGATTGGCCAGTACCAATGACAAGGGA
AGTATGGCTATGGAATCTGCCAAGGAGACTCGCTACTGTGCAGTGTGCAA
TGACTATGCTTCAGGCTACCATTATGGAGTCTGGTCCTGTGAGGGCTGCA
AGGCCTTCTTCAAGAGAAGTATTCAAGGACATAACGACTATATGTGTCCA
GCCACCAACCAGTGCACCATTGATAAAAACAGGAGGAAGAGCTGCCAGGC
CTGCCGGCTCCGCAAATGCTACGAAGTGGGAATGATGAAAGGTGGGATAC
GAAAAGACCGAAGAGGAGGGAGAATGTTGAAACACAAGCGCCAGAGAGAT
GATGGGGAGGGCAGGGGTGAAGTGGGGTCTGCTGGAGACATGAGAGCTGC
CAACCTTTGGCCAAGCCCGCTCATGATCAAACGCTCTAAGAAGAACAGCC
TGGCCTTGTCCCTGACGGCCGACCAGATGGTCAGTGCCTTGTTGGATGCT
GAGCCCCCCATACTCTATTCCGAGTATGATCCTACCAGACCCTTCAGTGA
AGCTTCGATGATGGGCTTACTGACCAACCTGGCAGACAGGGAGCTGGTTC
ACATGATCAACTGGGCGAAGAGGGTGCCAGGCTTTGTGGATTTGACCCTC
CATGATCAGGTCCACCTTCTAGAATGTGCCTGGCTAGAGATCCTGATGAT
TGGTCTCGTCTGGCGCTCCATGGAGCACCCAGGGAAGCTACTGTTTGCTC
CTAACTTGCTCTTGGACAGGAACCAGGGAAAATGTGTAGAGGGCATGGTG
GAGATCTTCGACATGCTGCTGGCTACATCATCTCGGTTCCGCATGATGAA
TCTGCAGGGAGAGGAGTTTGTGTGCCTCAAATCTATTATTTTGCTTAATT
CTGGAGTGTACACATTTCTGTCCAGCACCCTGAAGTCTCTGGAAGAGAAG
GACCATATCCACCGAGTCCTGGACAAGATCACAGACACTTTGATCCACCT
GATGGCCAAGGCAGGCCTGACCCTGCAGCAGCAGCACCAGCGGCTGGCCC
AGCTCCTCCTCATCCTCTCCCACATCAGGCACATGAGTAACAAAGGCATG
GAGCATCTGTACAGCATGAAGTGCAAGAACGTGGTGCCCCTCTATGACCT
GCTGCTGGAGATGCTGGACGCCCACCGCCTACATGCGCCCACTAGCCGTG
GAGGGGCATCCGTGGAGGAGACGGACCAAAGCCACTTGGCCACTGCGGGC
TCTACTTCATCGCATTCCTTGCAAAAGTATTACATCACGGGGGAGGCAGA
GGGTTTCCCTGCCACGGTCTGAGAGCTCCCTGGCTCCCACACGGTTCAGA
TAATCCCTGCTGCATTTTACCCTCATCATGCACCACTTTAGCCAAATTCT
GTCTCCTGCATACACTCCGGCATGCATCCAACACCAATGGCTTTCTAGAT
GAGTGGCCATTCATTTGCTTGCTCAGTTCTTAGTGGCACATCTTCTGTCT
TCTGTTGGGAACAGCCAAAGGGATTCCAAGGCTAAATCTTTGTAACAGCT
CTCTTTCCCCCTTGCTATGTTACTAAGCGTGAGGATTCCCGTAGCTCTTC
ACAGCTGAACTCAGTCTATGGGTTGGGGCTCAGATAACTCTGTGCATTTA
AGCTACTTGTAGAGACCCAGGCCTGGAGAGTAGACATTTTGCCTCTGATA
AGCACTTTTTAAATGGCTCTAAGAATAAGCCACAGCAAAGAATTTAAAGT
GGCTCCTTTAATTGGTGACTTGGAGAAAGCTAGGTCAAGGGTTTATTATA
GCACCCTCTTGTATTCCTATGGCAATGCATCCTTTTATGAAAGTGGTACA
CCTTAAAGCTTTTATATGACTGTAGCAGAGTATCTGGTGATTGTCAATTC
ATTCCCCCTATAGGAATACAAGGGGCACACAGGGAAGGCAGATCCCCTAG
TTGGCAAGACTATTTTAACTTGATACACTGCAGATTCAGATGTGCTGAAA
GCTCTGCCTCTGGCTTTCCGGTCATGGGTTCCAGTTAATTCATGCCTCCC
ATGGACCTATGGAGAGCAGCAAGTTGATCTTAGTTAAGTCTCCCTATATG
AGGGATAAGTTCCTGATTTTTGTTTTTATTTTTGTGTTACAAAAGAAAGC
CCTCCCTCCCTGAACTTGCAGTAAGGTCAGCTTCAGGACCTGTTCCAGTG
GGCACTGTACTTGGATCTTCCCGGCGTGTGTGTGCCTTACACAGGGGTGA
ACTGTTCACTGTGGTGATGCATGATGAGGGTAAATGGTAGTTGAAAGGAG
CAGGGGCCCTGGTGTTGCATTTAGCCCTGGGGCATGGAGCTGAACAGTAC
TTGTGCAGGATTGTTGTGGCTACTAGAGAACAAGAGGGAAAGTAGGGCAG
AAACTGGATACAGTTCTGAGGCACAGCCAGACTTGCTCAGGGTGGCCCTG
CCACAGGCTGCAGCTACCTAGGAACATTCCTTGCAGACCCCGCATTGCCC
TTTGGGGGTGCCCTGGGATCCCTGGGGTAGTCCAGCTCTTCTTCATTTCC
CAGCGTGGCCCTGGTTGGAAGAAGCAGCTGTCACAGCTGCTGTAGACAGC
TGTGTTCCTACAATTGGCCCAGCACCCTGGGGCACGGGAGAAGGGTGGGG
ACCGTTGCTGTCACTACTCAGGCTGACTGGGGCCTGGTCAGATTACGTAT
GCCCTTGGTGGTTTAGAGATAATCCAAAATCAGGGTTTGGTTTGGGGAAG
AAAATCCTCCCCCTTCCTCCCCCGCCCCGTTCCCTACCGCCTCCACTCCT
GCCAGCTCATTTCCTTCAATTTCCTTTGACCTATAGGCTAAAAAAGAAAG
GCTCATTCCAGCCACAGGGCAGCCTTCCCTGGGCCTTTGCTTCTCTAGCA
CAATTATGGGTTACTTCCTTTTTCTTAACAAAAAAGAATGTTTGATTTCC
TCTGGGTGACCTTATTGTCTGTAATTGAAACCCTATTGAGAGGTGATGTC
TGTGTTAGCCAATGACCCAGGTGAGCTGCTCGGGCTTCTCTTGGTATGTC
TTGTTTGGAAAAGTGGATTTCATTCATTTCTGATTGTCCAGTTAAGTGAT
CACCAAAGGACTGAGAATCTGGGAGGGCAAAAAAAAAAAAAAAGTTTTTA
TGTGCACTTAAATTTGGGGACAATTTTATGTATCTGTGTTAAGGATATGT
TTAAGAACATAATTCTTTTGTTGCTGTTTGTTTAAGAAGCACCTTAGTTT
GTTTAAGAAGCACCTTATATAGTATAATATATATTTTTTTGAAATTACAT
TGCTTGTTTATCAGACAATTGAATGTAGTAATTCTGTTCTGGATTTAATT
TGACTGGGTTAACATGCAAAAACCAAGGAAAAATATTTAGTTTTTTTTTT
TTTTTTTGTATACTTTTCAAGCTACCTTGTCATGTATACAGTCATTTATG
CCTAAAGCCTGGTGATTATTCATTTAAATGAAGATCACATTTCATATCAA
CTTTTGTATCCACAGTAGACAAAATAGCACTAATCCAGATGCCTATTGTT
GGATACTGAATGACAGACAATCTTATGTAGCAAAGATTATGCCTGAAAAG
GAAAATTATTCAGGGCAGCTAATTTTGCTTTTACCAAAATATCAGTAGTA
ATATTTTTGGACAGTAGCTAATGGGTCAGTGGGTTCTTTTTAATGTTTAT
ACTTAGATTTTCTTTTAAAAAAATTAAAATAAAACAAAAAAAAATTTCTA
GGACTAGACGATGTAATACCAGCTAAAGCCAAACAATTATACAGTGGAAG
GTTTTACATTATTCATCCAATGTGTTTCTATTCATGTTAAGATACTACTA
CATTTGAAGTGGGCAGAGAACATCAGATGATTGAAATGTTCGCCCAGGGG
TCTCCAGCAACTTTGGAAATCTCTTTGTATTTTTACTTGAAGTGCCACTA
ATGGACAGCAGATATTTTCTGGCTGATGTTGGTATTGGGTGTAGGAACAT
GATTTAAAAAAAAACTCTTGCCTCTGCTTTCCCCCACTCTGAGGCAAGTT
AAAATGTAAAAGATGTGATTTATCTGGGGGGCTCAGGTATGGTGGGGAAG
TGGATTCAGGAATCTGGGGAATGGCAAATATATTAAGAAGAGTATTGAAA
GTATTTGGAGGAAAATGGTTAATTCTGGGTGTGCACCAGGGTTCAGTAGA
GTCCACTTCTGCCCTGGAGACCACAAATCAACTAGCTCCATTTACAGCCA
TTTCTAAAATGGCAGCTTCAGTTCTAGAGAAGAAAGAACAACATCAGCAG
TAAAGTCCATGGAATAGCTAGTGGTCTGTGTTTCTTTTCGCCATTGCCTA
GCTTGCCGTAATGATTCTATAATGCCATCATGCAGCAATTATGAGAGGCT
AGGTCATCCAAAGAGAAGACCCTATCAATGTAGGTTGCAAAATCTAACCC
CTAAGGAAGTGCAGTCTTTGATTTGATTTCCCTAGTAACCTTGCAGATAT
GTTTAACCAAGCCATAGCCCATGCCTTTTGAGGGCTGAACAAATAAGGGA
CTTACTGATAATTTACTTTTGATCACATTAAGGTGTTCTCACCTTGAAAT
CTTATACACTGAAATGGCCATTGATTTAGGCCACTGGCTTAGAGTACTCC
TTCCCCTGCATGACACTGATTACAAATACTTTCCTATTCATACTTTCCAA
TTATGAGATGGACTGTGGGTACTGGGAGTGATCACTAACACCATAGTAAT
GTCTAATATTCACAGGCAGATCTGCTTGGGGAAGCTAGTTATGTGAAAGG
CAAATAGAGTCATACAGTAGCTCAAAAGGCAACCATAATTCTCTTTGGTG
CAGGTCTTGGGAGCGTGATCTAGATTACACTGCACCATTCCCAAGTTAAT
CCCCTGAAAACTTACTCTCAACTGGAGCAAATGAACTTTGGTCCCAAATA
TCCATCTTTTCAGTAGCGTTAATTATGCTCTGTTTCCAACTGCATTTCCT
TTCCAATTGAATTAAAGTGTGGCCTCGTTTTTAGTCATTTAAAATTGTTT
TCTAAGTAATTGCTGCCTCTATTATGGCACTTCAATTTTGCACTGTCTTT
TGAGATTCAAGAAAAATTTCTATTCTTTTTTTTGCATCCAATTGTGCCTG
AACTTTTAAAATATGTAAATGCTGCCATGTTCCAAACCCATCGTCAGTGT
GTGTGTTTAGAGCTGTGCACCCTAGAAACAACATATTGTCCCATGAGCAG
GTGCCTGAGACACAGACCCCTTTGCATTCACAGAGAGGTCATTGGTTATA
GAGACTTGAATTAATAAGTGACATTATGCCAGTTTCTGTTCTCTCACAGG
TGATAAACAATGCTTTTTGTGCACTACATACTCTTCAGTGTAGAGCTCTT
GTTTTATGGGAAAAGGCTCAAATGCCAAATTGTGTTTGATGGATTAATAT
GCCCTTTTGCCGATGCATACTATTACTGATGTGACTCGGTTTTGTCGCAG
CTTTGCTTTGTTTAATGAAACACACTTGTAAACCTCTTTTGCACTTTGAA
AAAGAATCCAGCGGGATGCTCGAGCACCTGTAAACAATTTTCTCAACCTA
TTTGATGTTCAAATAAAGAATTAAACTAAA
SEQ ID NO: 18 is a Progesterone Receptor amino
acid sequence (Genbank Accession No. AAD01587.1).
MTELKAKGPRAPHVAGGPPSPEVGSPLLCRPAAGPFPGSQTSDTLPEVSA
IPISLDGLLFPRPCQGQDPSDEKTQDQQSLSDVEGAYSRAEATRGAGGSS
SSPPEKDSGLLDSVLDTLLAPSGPGQSQPSPPACEVTSSWCLFGPELPED
PPAAPATQRVLSPLMSRSGCKVGDSSGTAAAHKVLPRGLSPARQLLLPAS
ESPHWSGAPVKPSPQAAAVEVEEEDGSESEESAGPLLKGKPRALGGAAAG
GGAAAVPPGAAAGGVALVPKEDSRFSAPRVALVEQDAPMAPGRSPLATTV
MDFIHVPILPLNHALLAARTRQLLEDESYDGGAGAASAFAPPRSSPCASS
TPVAVGDFPDCAYPPDAEPKDDAYPLYSDFQPPALKIKEEEEGAEASARS
PRSYLVAGANPAAFPDFPLGPPPPLPPRATPSRPGEAAVTAAPASASVSS
ASSSGSTLECILYKAEGAPPQQGPFAPPPCKAPGASGCLLPRDGLPSTSA
SAAAAGAAPALYPALGLNGLPQLGYQAAVLKEGLPQVYPPYLNYLRPDSE
ASQSPQYSFESLPQKICLICGDEASGCHYGVLTCGSCKVFFKRAMEGQHN
YLCAGRNDCIVDKIRRKNCPACRLRKCCQAGMVLGGRKFKKFNKVRVVRA
LDAVALPQPLGVPNESQALSQRFTFSPGQDIQLIPPLINLLMSIEPDVIY
AGHDNTKPDTSSSLLTSLNQLGERQLLSVVKWSKSLPGFRNLHIDDQITL
IQYSWMSLMVFGLGWRSYKHVSGQMLYFAPDLILNEQRMKESSFYSLCLT
MWQIPQEFVKLQVSQEEFLCMKVLLLLNTIPLEGLRSQTQFEEMRSSYIR
ELIKAIGLRQKGVVSSSQRFYQLTKLLDNLHDLVKQLHLYCLNTFIQSRA
LSVEFPEMMSEVIAAQLPKILAGMVKPLLFHKK
SEQ ID NO: 19 is an exemplary cDNA sequence
encoding a Progesterone Receptor (GenBank
Accession No. AF016381.1).
CTGACCAGCGCCGCCCTCCCCCGCCCCCGACCCAGGAGGTGGAGATCCCT
CCGGTCCAGCCACATTCAACACCCACTTTCTCCTCCCTCTGCCCCTATAT
TCCCGAAACCCCCTCCTCCTTCCCTTTTCCCTCCTCCCTGGAGACGGGGG
AGGAGAAAAGGGGAGTCCAGTCGTCATGACTGAGCTGAAGGCAAAGGGTC
CCCGGGCTCCCCACGTGGCGGGCGGCCCGCCCTCCCCCGAGGTCGGATCC
CCACTGCTGTGTCGCCCAGCCGCAGGTCCGTTCCCGGGGAGCCAGACCTC
GGACACCTTGCCTGAAGTTTCGGCCATACCTATCTCCCTGGACGGGCTAC
TCTTCCCTCGGCCCTGCCAGGGACAGGACCCCTCCGACGAAAAGACGCAG
GACCAGCAGTCGCTGTCGGACGTGGAGGGCGCATATTCCAGAGCTGAAGC
TACAAGGGGTGCTGGAGGCAGCAGTTCTAGTCCCCCAGAAAAGGACAGCG
GACTGCTGGACAGTGTCTTGGACACTCTGTTGGCGCCCTCAGGTCCCGGG
CAGAGCCAACCCAGCCCTCCCGCCTGCGAGGTCACCAGCTCTTGGTGCCT
GTTTGGCCCCGAACTTCCCGAAGATCCACCGGCTGCCCCCGCCACCCAGC
GGGTGTTGTCCCCGCTCATGAGCCGGTCCGGGTGCAAGGTTGGAGACAGC
TCCGGGACGGCAGCTGCCCATAAAGTGCTGCCCCGGGGCCTGTCACCAGC
CCGGCAGCTGCTGCTCCCGGCCTCTGAGAGCCCTCACTGGTCCGGGGCCC
CAGTGAAGCCGTCTCCGCAGGCCGCTGCGGTGGAGGTTGAGGAGGAGGAT
GGCTCTGAGTCCGAGGAGTCTGCGGGTCCGCTTCTGAAGGGCAAACCTCG
GGCTCTGGGTGGCGCGGCGGCTGGAGGAGGAGCCGCGGCTGTCCCGCCGG
GGGCGGCAGCAGGAGGCGTCGCCCTGGTCCCCAAGGAAGATTCCCGCTTC
TCAGCGCCCAGGGTCGCCCTGGTGGAGCAGGACGCGCCGATGGCGCCCGG
GCGCTCCCCGCTGGCCACCACGGTGATGGATTTCATCCACGTGCCTATCC
TGCCTCTCAATCACGCCTTATTGGCAGCCCGCACTCGGCAGCTGCTGGAA
GACGAAAGTTACGACGGCGGGGCCGGGGCTGCCAGCGCCTTTGCCCCGCC
GCGGAGTTCACCCTGTGCCTCGTCCACCCCGGTCGCTGTAGGCGACTTCC
CCGACTGCGCGTACCCGCCCGACGCCGAGCCCAAGGACGACGCGTACCTT
CTCTATAGCGACTTCCAGCCGCCCGCTCTAAAGATAAAGGAGGAGGAGGA
AGGCGCGGAGGCCTCCGCGCGCTCCCCGCGTTCCTACCTTGTGGCCGGTG
CCAACCCCGCAGCCTTCCCGGATTTCCCGTTGGGGCCACCGCCCCCGCTG
CCGCCGCGAGCGACCCCATCCAGACCCGGGGAAGCGGCGGTGACGGCCGC
ACCCGCCAGTGCCTCAGTCTCGTCTGCGTCCTCCTCGGGGTCGACCCTGG
AGTGCATCCTGTACAAAGCGGAGGGCGCGCCGCCCCAGCAGGGCCCGTTC
GCGCCGCCGCCCTGCAAGGCGCCGGGCGCGAGCGGCTGCCTGCTCCCGCG
GGACGGCCTGCCCTCCACCTCCGCCTCTGCCGCCGCCGCCGGGGCGGCCC
CCGCGCTCTACCCTGCACTCGGCCTCAACGGGCTCCCGCAGCTCGGCTAC
CAGGCCGCCGTGCTCAAGGAGGGCCTGCCGCAGGTCTACCCGCCCTATCT
CAACTACCTGAGGCCGGATTCAGAAGCCAGCCAGAGCCCACAATACAGCT
TCGAGTCATTACCTCAGAAGATTTGTTTAATCTGTGGGGATGAAGCATCA
GGCTGTCATTATGGTGTCCTTACCTGTGGGAGCTGTAAGGTCTTCTTTAA
GAGGGCAATGGAAGGGCAGCACAACTACTTATGTGCTGGAAGAAATGACT
GCATCGTTGATAAAATCCGCAGAAAAAACTGCCCAGCATGTCGCCTTAGA
AAGTGCTGTCAGGCTGGCATGGTCCTTGGAGGTCGAAAATTTAAAAAGTT
CAATAAAGTCAGAGTTGTGAGAGCACTGGATGCTGTTGCTCTCCCACAGC
CATTGGGCGTTCCAAATGAAAGCCAAGCCCTAAGCCAGAGATTCACTTTT
TCACCAGGTCAAGACATACAGTTGATTCCACCACTGATCAACCTGTTAAT
GAGCATTGAACCAGATGTGATCTATGCAGGACATGACAACACAAAACCTG
ACACCTCCAGTTCTTTGCTGACAAGTCTTAATCAACTAGGCGAGAGGCAA
CTTCTTTCAGTAGTCAAGTGGTCTAAATCATTGCCAGGTTTTCGAAACTT
ACATATTGATGACCAGATAACTCTCATTCAGTATTCTTGGATGAGCTTAA
TGGTGTTTGGTCTAGGATGGAGATCCTACAAACATGTCAGTGGGCAGATG
CTGTATTTTGCACCTGATCTAATACTAAATGAACAGCGGATGAAAGAATC
ATCATTCTATTCATTATGCCTTACCATGTGGCAGATCCCACAGGAGTTTG
TCAAGCTTCAAGTTAGCCAAGAAGAGTTCCTCTGTATGAAAGTATTGTTA
CTTCTTAATACAATTCCTTTGGAAGGGCTACGAAGTCAAACCCAGTTTGA
GGAGATGAGGTCAAGCTACATTAGAGAGCTCATCAAGGCAATTGGTTTGA
GGCAAAAAGGAGTTGTGTCGAGCTCACAGCGTTTCTATCAACTTACAAAA
CTTCTTGATAACTTGCATGATCTTGTCAAACAACTTCATCTGTACTGCTT
GAATACATTTATCCAGTCCCGGGCACTGAGTGTTGAATTTCCAGAAATGA
TGTCTGAAGTTATTGCTGCACAATTACCCAAGATATTGGCAGGGATGGTG
AAACCCCTTCTCTTTCATAAAAAGTGAATGTCATCTTTTTCTTTTAAAGA
ATTAAATTTTGTGG
SEQ ID NO: 20 is an exemplary oligo/polynucleotide
probe sequence.
TCTAGCCATTTGATGCCAACAGTAGAAAGGG
SEQ ID NO: 21 is an exemplary unique sequence
identifier sequence.
CTTGACTGAGCGACTGAGC
SEQ ID NO: 22 is another exemplary unique sequence
identifier sequence.
CTCCAGGGTTAGGCAGATC
SEQ ID NO: 23 is yet another exemplary unique
sequence identifier sequence.
CAGCGGGATAGTGCGATTC
SEQ ID NO: 24 is an exemplary oligo/polynucleotide
sequence that can be used in in situ polymerase
extension methods.
TAACTTACGGAGTCGCTCTACG
SEQ ID NO: 25 is an exemplary oligo/polynucleotide
sequence that can be used in in situ polymerase
extension.
GGATGGGATTCTTTAGGTCCTG
SEQ ID NO: 26 is an exemplary forward primer
sequence comprising an NGS adaptor sequence for
use in in situ polymerase extension.
AATGATACGGCGACCACCGAGATCTACACTCTTTCCCTACACGACGCTCT
TCCGATCT
SEQ ID NO: 27 is an exemplary reverse primer
sequence comprising an NGS adaptor sequence for
use in in situ polymerase extension.
CAAGCAGAAGACGGCATACGAGATCGGTCTCGGCATTCCTGCTGAACCGC
TCTTCCGATCT
SEQ ID NO: 28 is an exemplary primer sequence that
can be used for PCR analysis of the SEQ ID NO: 27.
CGACCACCGAGATCTAC
SEQ ID NO: 29 is an exemplary reverse primer that
can be used in PCR analysis of isolated probes
that have been extended using in situ polymerase
extension.
ACGTGAACTTTGAAAGG
SEQ ID NO: 30 is an exemplary reverse primer that
can be used in PCR analysis of isolated probes
that have been extended using in situ polymerase
extension.
GTAGATCCTGCAAAAAGAGTG
SEQ ID NO: 31 is an exemplary reverse primer that
can be used in PCR analysis of isolated probes
that have been extended using in situ polymerase
extension.
CCTTTCTACTGTTGGCATC
SEQ ID NO: 32 is an exemplary reverse primer that
can be used in PCR analysis of isolated probes
that have been extended using in situ polymerase
extension.
CTGAGAATGATTCTGTCTGG
SEQ ID NO: 33 is an exemplary reverse primer that
can be used in PCR analysis of isolated probes
that have been extended using in situ polymerase
extension.
CAATGCGGTCCATATATCC
SEQ ID NO: 34 is an exemplary oligo/polynucleotide
sequence comprising a detectable label, an oligo/
polynucleotide sequence, and photocleavable
moiety.
{Biotin}/CATTCAAATCCCCGAGTTGAACTTTCCTTTCAAAGTTCAC
GT/{PCBiotin}
SEQ ID NO: 35 is an exemplary oligo/polynucleotide
sequence comprising a detectable label, an oligo/
polynucleotide sequence, and photocleavable
moiety.
{Biotin}/TCTAGCCATTTGATGCCAACAGTAGAAAGGG/
{PCBiotin}
SEQ ID NO: 36 is an exemplary oligo/polynucleotide
sequence that can be labeled with a photocleavable
moiety for use in the methods disclosed herein.
{PCBiotin}/TAACTTACGGAGTCGCTCTACGCATTCAAATCCCCGAGT
TGAACTTTCCTTTCAAAGTTCACGT
SEQ ID NO: 37 is an exemplary oligo/polynucleotide
sequence that can be labeled with a photocleavable
moiety for use in the methods disclosed herein.
{PCBiotin}TAACTTACGGAGTCGCTCTACG
TCTAGCCATTTGATGCCAACAGTAGAAAGGG
SEQ ID NO: 38 is an exemplary oligo/polynucleotide
sequence that is modified to comprise a caged
thymidine and further comprises a biotin moiety.
{Biotin}TAACTTACGGAGTCGCTCTACGCATTCAAATCCCCGAGTTGA
ACTTTCCTTTCAAAGTTCACGT
SEQ ID NO: 39 is an exemplary oligo/polynucleotide
sequence that is modified to comprise a caged
thymidine and further comprises a biotin moiety.
{Biotin}/TAACTTACGGAGTCGCTCTACGCATTCAAATCCCCGAGTTG
AACTTTCCTTTCAAAGTTCACGTT
SEQ ID NO: 40 is an exemplary oligo/polynucleotide
sequence that is modified to comprise a caged
thymidine and further comprises a biotin moiety.
{Biotin}/TAACTTACGGAGTCGCTCTACGTCTAGCCATTTGATGCCAA
CAGTAGAAAGGGAAATA
SEQ ID NO: 41 is an exemplary oligo/polynucleotide
sequence that is modified to comprise a caged
thymidine and further comprises a biotin moiety.
{Biotin}/TAACTTACGGAGTCGCTCTACGTCTAGCCATTTGATGCCAA
CAGTAGAAAGGGAAATATCTT
SEQ ID NO. 42: is an exemplary primer sequence
(PS1) according to Example 9.
5′-TAACTTACGGAGTCGCTCTACG-3′
SEQ ID NO. 43: is another exemplary sequence for
Chr7 according to Example 9.
5′-TCTAGCCATTTGATGCCAACAGTAGAAAGGG-3′
SEQ ID NO. 44 is a Biotin-PS1-CHR7LAN5C probe
according to Example 9.
5′-
{Biotin}/TAACTTACGGAGTCGCTCTACGTCTAGCCATTTGATGCCAA
CAGTAGAAAGGG-3′
SEQ ID NO. 45 is a probe according to Example 7
(PE1.0-CHR7).
5′AATGATACGGCGACCACCGAGATCTACACTCTTTCCCTACACGACGCT
CTTCCGATCTTCTAGCCATTTGATGCCAACAGTAGAAAGGG-3′
SEQ ID NO. 46 is a probe according to Example 7
(PE1.0-CHR17).
5′AATGATACGGCGACCACCGAGATCTACACTCTTTCCCTACACGACGCT
CTTCCGATCTCATTCAAATCCCCGAGTTGAACTTTCCTTTCAAAGTTCAC
GT-3′
SEQ ID NO. 47 is a BiotinHER2Exon10 probe
according to Example 10, wherein *T denotes
positions of caged thymidines in caged versions of
the probes:
5′-{Biotin}/TAACTTACGGAGTC
GCTCTACGGCATGGAGCACT*T*GCGAGAGGT*GAGGGCAGT*T*A-3′
SEQ ID NO.48 is a BiotinBRAFExon13 probe according
to Example 10, wherein *T denotes positions of
caged thymidines in caged versions of the probes.
5′-{Biotin}/TAACTTACGGAGTCGCTCTACG
AGTGGTGTGAGGGCTCCAGCTIGTAT*CACCAT*CT*AT*T*G-3′

We claim:

1. A method for performing contextual molecular diagnostics, comprising:
contacting a sample with a probe comprising a photo-cleavable moiety, a specific binding moiety to a target, and a detection tag using conditions sufficient to facilitate binding of the specific binding moiety to a target in the sample;
removing probe that does not bind to the sample;
selecting a region of the sample for irradiation based on contextual information;
irradiating the selected region of the sample with light of a wavelength and an intensity sufficient to cleave the photo-cleavable moiety, thereby freeing the detection tag from the selected region of the sample; and
detecting the detection tag.

2. The method of claim 1, wherein
contacting includes contacting the sample with multiple probes having photo-cleavable moieties and specific binding moieties to multiple targets, the multiple different probes comprising unique detection tags and unique specific binding moieties and
detecting the detection tag includes detecting the multiple unique detection tags.

3. The method of claim 1, wherein selecting further comprises selecting additional regions; the method further comprising:
iteratively and separately irradiating the additional regions.

4. The method of claim 3, the method further comprising iteratively depositing the detection tags from the additional regions in additional separate reaction vials.

5. The method claim 1, wherein the specific binding moiety is selected from an amino acid, a peptide, a protein, an antibody, a nucleotide, an oligonucleotide, a polynucleotide, a primer, an aptamer, a binding variable region, a plasmid, DNA, RNA, miRNA, or combinations thereof.

6. The method of claim 1, wherein the method further comprises:
irradiating a second selected region of the sample to cleave the photo-cleavable moiety, thereby freeing the detection tag from the second selected region of the sample; and
detecting the detection tag that was bound to the target within the second selected region of the sample.

7. The method claim 1, wherein the detection tag is a unique sequence identifier or a tag sequence.

8. The method of claim 7, wherein the unique sequence identifier comprises a reagent barcode, an adapter sequence, an alignment sequence, a subject index sequence, or combinations thereof.

9. The method of claim 8, wherein the reagent barcode comprises from about 6 nucleotides to about 200 nucleotides, the adapter sequence comprises from about 5 nucleotides to about 50 nucleotides, the alignment sequence comprises from about 3 nucleotides to at least about 20 nucleotides, and the subject index sequence comprises from at least 5 nucleotides to about 10 nucleotides.

10. The method of claim 1, wherein the detection tag further comprises a detectable label.

11. The method of claim 10, wherein the detectable label is a hapten.

12. The method of claim 11, wherein the method further includes purifying the detection tag using by binding the hapten to an anti-hapten anti-body.

13. The method of claim 1, wherein the probe further comprises at least one linker that links two probe components.

14. The method of claim 1, wherein the sample is irradiated with light for a time sufficient to cleave the photo-cleavable moiety.

15. The method of claim 1, wherein the conditions sufficient to bind the specific binding moiety of the probe to the sample are suitable for inducing hybridization and/or chemical coupling of the probe to the sample.

16. The method claim 1, wherein selecting the region of the sample comprises manually marking one or more portions of the sample or digitally marking one or more portions of the sample.

17. The method claim 1, wherein the method further comprises contacting the sample with a visually detectable reagent and selecting includes the contextual information provided by the visually detectable reagent.

18. The method of claim 17, wherein the detectable reagent includes a fluorescent or chromogenic reagent.

19. The method of claim 1, wherein detection of the detection tag further comprises sequencing the detection tag using next generation sequencing, analyzing the detection tag using PCR, or analyzing the detection tag using an array platform.

20. The method of claim 1, further comprising contacting the sample with a second probe comprising a second specific binding moiety, a second photo-cleavable moiety, and a second detection tag.

21. The method claim 1, wherein selecting further comprises selecting a control region; the method further comprising:
separately irradiating the control region and
separately detecting whether the detection tag is present in the control region.

22. A method for performing contextual molecular diagnostics, the method comprising:
contacting a sample with a probe comprising a photo-reactive moiety, a specific binding moiety, and a detection tag using conditions sufficient to facilitate binding of the specific binding moiety to a target in the sample;
removing probe that does not bind to the sample;
selecting a region of the sample for irradiation based on contextual information;
irradiating the selected region of the sample with light of a wavelength and an intensity sufficient to cause the photo-reactive moiety to react, thereby freeing the detection sequence for further reaction;
exposing the tissue sample to an enzyme that acts on the detection sequence to effect a change; and
detecting the change.

23. The method of claim 22, wherein the detection tag is an extendable primer, the photo-reactive moiety is a photo-cleavable blocking group, the enzyme is a polymerase, and the change is an extension of a primer sequence in the presence of dATP, dCTP, dTTP, and dGTP.

24. The method of claim 22, wherein the photo-cleavable moiety includes a caged-ATP molecule.

25. The method of claim 22, wherein the specific binding moiety is selected from an amino acid, a peptide, a protein, an antibody, a nucleotide, an oligonucleotide, a polynucleotide, a primer, an aptamer, a binding variable region, a plasmid, DNA, RNA, miRNA, or combinations thereof.

26. The method of claim 22, wherein the method further comprises:
irradiating a second selected region of the sample to cause the photo-reactive moiety to react, thereby freeing the detection sequence for further reaction from the second selected region of the sample; and
detecting the detection tag that was bound to the target within the second selected region of the sample.

27. A system for performing contextual diagnostics using a photo-activated probe, comprising:
a slide configured to accept a sample;
a vessel containing the photo-activated probe;
a slide imaging device configured to receive the slide, and
a light source configured to selectively irradiate the sample.

28. The system of claim 27, wherein the photo-activated probe comprises a specific binding moiety, a photo-activated moiety, and a detection tag, wherein the photo-activated probe is configured to be detectable upon selective irradiation.

29. The system of any of claim 28, further comprising:
a dispensing instrument configured to dispense an elution buffer onto the slide thereby facilitating isolation of the detection tag; and
a recovery instrument configured to recover the detection tag.

30. The system of any of claim 28, further comprising:
a sequencing instrument configured to receive and sequence the detection tag; and
a computer configured to translate information generated from the sequencing instrument to the digital screen, thereby correlating the detection tag to the selected region from which it was cleaved.

31. A kit for performing contextual diagnostics, comprising a probe reagent solution comprising a specific binding moiety, a photo-cleavable moiety, and a detection tag.
